(19) United States
(12) Patent Application Publication
(10) Pub. No.: US 2005/0287638 A1
(43) Pub. Date: Dec. 29, 2005

(54) Hyaluronan receptor for endocytosis, variants thereof, and methods of making and using same

(76) Inventors: Paul H. Weigel, Edmond, OK (US); Janet Weigel, Edmond, OK (US)

Correspondence Address: DUNLAP, CODDING & ROGERS P.C., PO BOX 16370, OKLAHOMA CITY, OK, 73113, US

(21) Appl. No.: 11/128,059

(22) Filed: May 12, 2005

Related U.S. Application Data
(63) Continuation-in-part of application No. 10/133,172, filed on Apr. 25, 2002.
(63) Continuation-in-part of application No. 09/842,930, filed on Apr. 25, 2001.
(60) Provisional application No. 60/570,915, filed on May 13, 2004.
(60) Provisional application No. 60/286,468, filed on Apr. 25, 2001.
(60) Provisional application No. 60/199,538, filed on Apr. 25, 2000.

(30) Foreign Application Priority Data
Apr. 25, 2001 (AU) 2001253805
Apr. 25, 2001 (CA) 2,407,415
Apr. 25, 2001 (EP) 01927344.0
Apr. 25, 2001 (WO) PCT/US01/13403

(51) Int. Cl. C12Q 1/68; C07K 14/705; C07H 21/04

(52) U.S. Cl. 435/69.1; 435/320.1; 435/325; 530/350; 536/23.5

(57) ABSTRACT

A purified recombinant mammalian HARE comprising a polypeptide which is able to specifically bind at least one of HA, chondroitin and chondroitin sulfate is disclosed, as well as methods of expressing and using same. Also disclosed are functionally active variants of HARE which are able to specifically bind at least one of HA, chondroitin and chondroitin sulfate, as well as methods of expressing and using same.

### CROSS-REFERENCE TO RELATED APPLICATIONS

This application claims priority under 35 U.S.C. 119(e) of provisional application U.S. Ser. No. 60/570,915, filed May 13, 2004; the contents of which are hereby expressly incorporated herein by reference.

This application is also a continuation-in-part of U.S. Ser. No. 10/133,172, filed Apr. 25, 2002; which claims priority under 35 U.S.C. 119(e) of provisional application U.S. Ser. No. 60/286,468, filed Apr. 25, 2001. Said U.S. Ser. No. 10/133,172 is also a continuation-in-part of U.S. Ser. No. 09/842,930, filed Apr. 25, 2001, which claims priority under 35 U.S.C. 119(e) of provisional application U.S. Ser. No. 60/199,538, filed Apr. 25, 2000. The contents of each of the above-referenced patent applications are hereby expressly incorporated herein in their entirety by reference.

### FEDERALLY SPONSORED RESEARCH OR DEVELOPMENT

The government owns certain rights in the present invention pursuant to a grant from the National Institutes of Health (GM 35978).

### BACKGROUND OF THE INVENTION

1. Field of the Invention

The present invention generally relates to a Hyaluronan (“HA”) Receptor for Endocytosis (HARE), variants thereof, and antibodies against HARE, and more particularly, but not by way of limitation, to methods of targeting compounds to cells and preventing interactions between cells by utilizing HARE, variants thereof and/or such antibodies.

2. Brief Description of the Related Art

HA, also referred to herein as hyaluronic acid, or hyaluronan, is a glycosaminoglycan (GAG) composed of the repeating disaccharide β(1,4)-D-glucuronic acid-(β1,3)-N-acetyl-D-glucosamine. HA is an important and often abundant extracellular matrix (ECM) component of all tissues, in particular cartilage, skin and vitreous humor (Evered and Whelan, (1989)). Although HA is ubiquitous throughout the body, it is particularly enriched in tissues that require its unique physical properties, e.g., in joint synovial fluid (where it serves as a lubricant; Hills, 2000), the vitreous humor of the eye (where the turgor pressure it creates maintains the shape of the eye; Meyer and Palmer, 1934; and Hollyfield, 1999), the skin and to a lesser extent in connective tissues (where it enhances elasticity and cohesive properties; Lamberg and Stoolmiller, 1974; and Knudson and Knudson, 2001). In addition to its physical roles as an important structural molecule in the ECM, HA is also able to modulate, or is required for, complex cellular behaviors such as cell migration (Itano et al., 2002; and Evanko et al., 1999), angiogenesis (West et al., 1985; Slevin et al., 1998; and Rahmanian et al., 1997), cell signaling (Oliferenko et al., 2000), wound healing (Weigel et al., 1986; Laurent et al., 1988; Burd et al., 1991; and Haney and Doty, 1998), oocyte maturation (Kimura et al., 2002), and development (Camenisch et al., 2002). The HA field has been energized in recent years by the recognition that small HA oligosaccharides behave as ligands that alter gene expression patterns in responsive cells (West et al., 1985; Slevin et al., 1998; Rahmanian et al., 1997; and Ghatak et al., 2005). The biology of HA now encompasses a wider array of cellular behaviors. For example, small HA oligosaccharides can make tumor cells more sensitive to chemotherapeutic drugs by altering the cellular signaling cascades generated by CD44 (Misra et al., 2003).

The average adult human contains ˜15 g of HA, of which ˜5 g is synthesized and degraded daily in tissues throughout the body (Laurent and Fraser, 1992). Although local turnover of HA occurs in avascular tissues, particularly cartilage (Aguiar et al., 1999), two major clearance systems are responsible for HA degradation and removal in the body (Laurent and Fraser, 1992). The first is the lymphatic system, which accounts for about 85% of the HA turnover, and the second is in the liver, which accounts for the other approximately 15% of the total body HA turnover.

Throughout the body, HA is continuously synthesized and degraded in almost all tissues. At the same time, chondroitin sulfate and other glycosaminoglycans are also released from the cleavage of proteoglycans, especially aggregating proteoglycans associated with HA. Large native HA molecules (about 10⁷ Da) are partially degraded into large fragments (about 10⁶ Da) that are released from the matrix and enter the lymphatic system, thereafter flowing to lymph nodes.

Due to the rapid turnover rate of HA (Tammi et al., 1991), the body requires an efficient way to bind, internalize, and catabolize HA during this normal turnover process. Although there are several molecules that specifically bind to HA, such as CD44 (Gee et al., 2004), RHAMM (Lynn et al., 2001), and LYVE-1 (Banerji et al., 1999), the Hyaluronic Acid Receptor for Endocytosis (HARE), which was first recognized more than 20 years ago (Fraser et al., 1981; and Fraser et al., 1983), is the receptor that mediates systemic clearance of HA. HARE both binds and internalizes HA via the coated pit pathway (Zhou et al., 2002; Smedsrod et al., 1988; Harris et al., 2004; and Weigel and Yik, 2002).

In mammals, large HA molecules diffuse from the tissues into the lymphatic system where most of the HA (˜85%) is taken up by the sinusoidal endothelial cells of the lymph nodes (Laurent and Fraser, 1992; and Weigel and Yik, 2002). The remaining smaller HA molecules enter the blood stream and are taken up primarily by the sinusoidal endothelial cells of the liver (Fraser et al., 1981; and Fraser et al., 1983). Failure to remove and break down HA in humans could cause an increase in osmotic pressure of the blood (Laurent and Fraser, 1992). Additionally, physiological conditions such as rheumatoid arthritis (Manicourt et al., 1999), cirrhosis (Lai et al., 1998), scleroderma (Freitas et al., 1996), and some cancers (Thylen et al., 1999) are associated with elevated HA levels in the blood.

In addition to the normal turnover of HA in tissues throughout the body, a wide range of biomedical and clinical applications use exogenous HA that is also removed from the lymphatics or ultimately from the blood and degraded by the LEC HARE. For example, HA is used extensively in eye surgery, in the treatment of joint diseases including osteoarthritis, and is being developed as a drug delivery vehicle. Numerous studies have explored the benefit of HA during wound healing. The exogenous HA introduced in these various applications is naturally degraded by the lymph and LEC systems noted above.

In the parent applications U.S. Ser. Nos. 10/133,172 and 09/842,930, which have previously been incorporated herein by reference, the identification, recombinant expression and purification of a rat isoform of HARE was described, as well as characterization of the GAG specificities of the rat HARE. The parent applications disclose the identification of monoclonal antibodies (mAbs) directed against the rat 175 kDa HARE and inhibition of HA endocytosis by such mAbs in rat LECs as well as cells expressing the recombinant 175 kDa rat HARE. The parent applications also disclose the use of the mAbs for identifying immunocyochemical localization of HARE in human liver, spleen, lymph node and bone marrow, and the purification of 190 kDa and 315 kDa human HARE. In addition, a putative human isoform of HARE was also described in the parent applications; however, prior to the present invention, no human HARE isoform has been recombinantly expressed in stable cell lines, and therefore the GAG specificity and endocytic activity of the small hHARE isoform has not been studied in the absence of the larger hHARE isoform.

While the rat HARE proteins have been studied in isolated rat LECs, as described in the parent applications, no cellular studies of the human HARE proteins have been possible. Human LECs are not available commercially, and to date, no cell lines have been identified that express either the 190 kDa or 315 kDa hHARE isoforms. Consequently, very little is known about the GAG specificity or function of human HARE. Further, it has not been possible to identify variants of human HARE proteins prior to the present invention.

Therefore, there exists a need in the art for isolation and recombinant expression of a human HA receptor for endocytosis (HARE), the identification of splice variants thereof, as well as antibodies directed thereto, and methods of targeting compounds to cells and preventing interactions between cells by utilizing HARE and/or such antibodies.

### SUMMARY OF THE INVENTION

The present invention is related to recombinant mammalian HARE, variants thereof and fragments thereof, such as a soluble form of HARE, that are capable of specifically binding at least one of HA, chondroitin and chondroitin sulfate.

In one embodiment, the present invention is related to a purified recombinant mammalian HARE comprising a polypeptide which is able to specifically bind at least one of HA, chondroitin and chondroitin sulfate. The purified recombinant mammalian HARE comprises at least one of: a purified recombinant mammalian HARE having a molecular weight of about 190 kDa; a purified recombinant mammalian HARE having a molecular weight of about 315 kDa; a purified recombinant mammalian HARE having an amino acid sequence in accordance with SEQ ID NO:4; a purified recombinant mammalian HARE having an amino acid sequence in accordance with SEQ ID NO:96; a purified recombinant human HARE; and a purified recombinant mammalian HARE which is recognized by at least one of the monoclonal antibodies mAb-30, mAb-154, mAb-159 and a monoclonal antibody which demonstrates an immunological binding characteristic of such monoclonal antibodies.

The present invention is also related to a method of producing a recombinant, functionally active mammalian HARE wherein the recombinant, functionally active HARE is able to specifically bind at least one of HA, chondroitin and chondroitin sulfate. In the method, a recombinant host cell containing a recombinant DNA segment which encodes and is capable of expressing the recombinant mammalian HARE described above is provided, and the recombinant host cell is cultured under conditions that allow for expression of the recombinant DNA segment encoding the functionally active, recombinant mammalian HARE, thereby producing recombinant, functionally active mammalian HARE which is able to specifically bind at least one of HA, chondroitin and chondroitin sulfate. The method may further comprise the step of separating and purifying the recombinant, functionally active mammalian HARE from the recombinant host cell.

In another embodiment, the present invention is related to an isolated nucleic acid sequence encoding a functionally active mammalian HARE which is able to specifically bind at least one of HA, chondroitin and chondroitin sulfate, the isolated nucleic acid sequence comprising a nucleic acid sequence in accordance with SEQ ID NO:95. The present invention is also related to a recombinant vector selected from the group consisting of a plasmid, cosmid, phage, and virus vector, wherein the recombinant vector further comprises such isolated nucleic acid sequence encoding a functionally active mammalian HARE. The recombinant vector may be an expression vector which may comprise a promoter operatively linked to the coding region of the mammalian HARE. The recombinant vector may be introduced into a recombinant host cell by transfection, electroporation and/or transduction, such as a eucaryotic cell, and the recombinant host cell produces a functionally active mammalian HARE which specifically binds and endocytoses at least one of HA, chondroitin and chondroitin sulfate. The purified nucleic acid sequence may be integrated into a chromosome of the recombinant host cell.

The present invention is also related to a method of producing a functionally active mammalian HARE which is able to specifically bind at least one of HA, chondroitin and chondroitin sulfate. The method includes providing the recombinant host cell described herein above, wherein the recombinant host cell is capable of expressing a functionally active mammalian HARE, and culturing the recombinant host cell under conditions that allow for expression of the purified nucleic acid sequence encoding a functionally active mammalian HARE, thereby producing a functionally active mammalian HARE which is able to specifically bind at least one of HA, chondroitin and chondroitin sulfate. The method may further include the step of separating and purifying the functionally active mammalian HARE from the recombinant host cell.

In another embodiment, the present invention is related to an isolated nucleic acid sequence encoding a functionally active variant or fragment of HARE, wherein the functionally active variant or fragment of HARE is able to specifically bind at least one of HA, chondroitin and chondroitin sulfate. The nucleic acid sequence comprises at least one of: a nucleic acid sequence in accordance with at least one of SEQ ID NO:55, SEQ ID NO:57, SEQ ID NO:59, SEQ ID NO:61, SEQ ID NO:73, SEQ ID NO:75, SEQ ID NO:77, SEQ ID NO:79, and SEQ ID NO:81; a nucleic acid sequence which will hybridize to a complement of at least one of the nucleic acid sequences listed above or a fragment thereof under stringent hybridization conditions; a nucleic acid sequence that has at least about 76%, 80%, 85% or 90% sequence identity to at least one of the nucleic acid sequences listed above; a nucleic acid sequence that encodes semiconservative or conservative amino acid changes when compared to at least one of the nucleic acid sequences listed above; and a nucleic acid sequence which but for the degeneracy of the genetic code, or encoding of functionally equivalent amino acids, would hybridize to at least one of the nucleic acid sequences listed above. The functionally active variant or fragment of HARE encoded by the isolated nucleic acid sequence may be soluble.

The present invention is also related to a recombinant vector selected from the group consisting of a plasmid, cosmid, phage, and virus vector, wherein the recombinant vector further comprises the purified nucleic acid sequence encoding a functionally active variant or fragment of HARE described herein above. The recombinant vector may be an expression vector, which may include a promoter operatively linked to the coding region of the HARE variant or fragment. The recombinant vector may be introduced into a recombinant host cell by transfection, electroporation and/or transduction, such as a eucaryotic cell, and the recombinant host cell produces a functionally active variant or fragment of HARE which specifically binds and endocytoses at least one of HA, chondroitin and chondroitin sulfate. The purified nucleic acid sequence may be integrated into a chromosome of the recombinant host cell.

The present invention is also related to a method of producing a functionally active variant or fragment of HARE wherein the functionally active variant or fragment of HARE is able to specifically bind at least one of HA, chondroitin and chondroitin sulfate. The method includes providing the recombinant host cell described herein above, wherein the recombinant host cell is capable of expressing a functionally active variant or fragment of HARE, and culturing the recombinant host cell under conditions that allow for expression of the purified nucleic acid sequence encoding a functionally active variant or fragment of HARE, thereby producing a functionally active variant or fragment of HARE which is able to specifically bind at least one of HA, chondroitin and chondroitin sulfate. The method may further include the step of separating and purifying the functionally active variant or fragment of HARE from the recombinant host cell.

In yet another embodiment, the present invention is related to a purified recombinant mammalian HARE variant or fragment comprising a polypeptide which is able to specifically bind at least one of HA, chondroitin and chondroitin sulfate. The purified recombinant mammalian HARE variant or fragment comprises at least one of: a soluble fragment of HARE; an amino acid sequence in accordance with at least one of SEQ ID NO:56, SEQ ID NO:58, SEQ ID NO:60, SEQ ID NO:62, SEQ ID NO:74, SEQ ID NO:76, SEQ ID NO:78, SEQ ID NO:80, and SEQ ID NO:82; an amino acid sequence encoded by a nucleic acid sequence which will hybridize to a complement of a nucleic acid sequence that encodes at least one of the amino acid sequences listed above or a fragment thereof under stringent hybridization conditions; an amino acid sequence that has at least about 76%, 80%, 85% or 90% sequence identity to at least one of the amino acid sequences listed above; and an amino acid sequence that has semiconservative or conservative amino acid changes when compared to at least one of the amino acid sequences listed above.

The present invention is also related to a method of producing a functionally active variant or fragment of HARE wherein the functionally active variant or fragment of HARE is able to specifically bind at least one of HA, chondroitin and chondroitin sulfate. The method includes providing a recombinant host cell containing a recombinant DNA segment which encodes and is capable of expressing the recombinant mammalian HARE variant or fragment described herein above, and culturing the recombinant host cell under conditions that allow for expression of the recombinant DNA segment encoding a recombinant mammalian HARE variant or fragment, thereby producing a recombinant, functionally active mammalian HARE variant or fragment which is able to specifically bind at least one of HA, chondroitin and chondroitin sulfate. The method may further include the step of separating and purifying the recombinant, functionally active, soluble mammalian HARE variant or fragment from the recombinant host cell.

In yet another embodiment, the present invention is related to a kit for determining the presence of at least one of HA, heparin, CS-A, CS-B, CS-C, CS-D, CS-E, chondroitin, keratan sulfate, and heparan sulfate. The kit includes at least one variant or fragment of HARE, wherein the at least one variant or fragment of HARE is capable of selectively binding at least one of HA, heparin, CS-A, CS-B, CS-C, CS-D, CS-E, chondroitin, keratan sulfate, and heparan sulfate and does not bind at least one of HA, heparin, CS-A, CS-B, CS-C, CS-D, CS-E, chondroitin, keratan sulfate, and heparan sulfate. The kit may further include a second variant of fragment of HARE, wherein the second variant or fragment of HARE is capable of binding at least one of heparin, CS-A, CS-B, CS-C, CS-D, CS-E, chondroitin, keratan sulfate, and heparan sulfate and does not bind at least one of heparin, CS-A, CS-B, CS-C, CS-D, CS-E, chondroitin, keratan sulfate, and heparan sulfate, and wherein the two variants' inability to bind at least one of heparin, CS-A, CS-B, CS-C, CS-D, CS-E, chondroitin, keratan sulfate, and heparan sulfate is different.

The present invention also relates to methods of using HA, HARE or a fragment or variant thereof and/or a monoclonal antibody raised against a portion of HARE, such as but not limited to, an HA-binding domain of HARE, a chondroitin-binding domain of HARE and/or a chondroitin sulfate-binding domain of HARE, to target compounds to specific cells or to prevent interactions between two types of cells.

In one embodiment, the present invention relates to a method of targeting a compound to a tissue of an individual wherein cells of the tissue express a functionally active HARE or a variant or fragment thereof. The compound is conjugated to at least one of HA, chondroitin, chondroitin sulfate, and a monoclonal antibody that selectively binds to an epitope of HARE. An effective amount of the complex formed of compound conjugated to HA-, chondroitin-, chondroitin sulfate-, or HARE monoclonal antibody can then be administered to the individual. The compound may be, for example, a chemotherapeutic agent or a radioisotope, or the compound may be deleterious to cells in close proximity to the cells expressing HARE (or a variant or fragment thereof) on a surface thereof upon delivery of the compound to the cells expressing HARE (or a variant or fragment thereof.

In another embodiment, the present invention relates to a method of inhibiting interaction between a first cell expressing HARE or a variant or fragment thereof on a surface thereof and a second cell having at least one of HA, chondroitin and chondroitin sulfate on a surface thereof. An effective amount of a compound that inhibits binding of at least one of HA, chondroitin and chondroitin sulfate to HARE (or a variant or fragment thereof), such as a mimetic peptide or a monoclonal antibody that selectively binds to an epitope of HARE (or a variant or fragment thereof) and inhibits binding of at least one of HA, chondroitin and chondroitin sulfate to HARE (or a variant or fragment thereof), is administered to prevent such interaction.

Optionally, the method of inhibiting interaction between a first cell expressing HARE on a surface thereof and a second cell whose surface contains at least one of HA, chondroitin and chondroitin sulfate may include providing a functionally active, soluble variant or fragment of HARE capable of binding at least one of HA, chondroitin and chondroitin sulfate on the surface of the second cell. Then, an effective amount of the functionally active, soluble variant or fragment of HARE is administered, wherein the functionally active, soluble variant or fragment of HARE inhibits binding of HARE expressed on the surface of the first cell to at least one of HA, chondroitin and chondroitin sulfate on the surface of the second cell.

In yet another embodiment, the present invention includes a method of targeting a compound to a cell of an individual wherein the cell does not express a functionally active HARE on a surface thereof by administering an effective amount of a monoclonal antibody that binds HARE or a variant or fragment thereof and blocks binding of at least one of HA, chondroitin and chondroitin sulfate to the HARE or fragment or variant thereof. The compound can then be conjugated to at least one of HA, chondroitin and chondroitin sulfate, and an effective amount of the conjugate can be administered to the individual such that the compound is targeted to a cell that expresses at least one cell surface or extracellular matrix component capable of binding at least one of HA, chondroitin and chondroitin sulfate.

In yet another embodiment of the present invention, methods of detecting at least one of HA, chondroitin and chondroitin sulfate in a sample, as well as quantitating the presence of each of HA, chondroitin and chondroitin sulfate, are provided. A HARE protein, peptide fragment or variant thereof containing at least one of an HA-, a chondroitin-, and a chondroitin sulfate-binding domain is provided and may be immobilized on a solid support. The sample is then contacted with the HARE protein, peptide fragment or variant thereof to form a mixture, whereby at least one of HA, chondroitin and chondroitin sulfate present in the sample binds to the HARE protein, peptide fragment or variant thereof. Unbound sample is then washed away, and the HA, chondroitin or chondroitin sulfate bound to the HARE protein, peptide fragment or variant thereof may be detected by one of two ways. First, at least one of labeled HA, labeled chondroitin and labeled chondroitin sulfate is contacted with the mixture, and a determination that at least one of HA, chondroitin and chondroitin sulfate is present in the sample is made if the labeled HA, chondroitin or chondroitin sulfate does not bind or has decreased binding to the HARE protein, peptide fragment or variant thereof. Second, a labeled HARE protein, peptide fragment or variant thereof containing at least one of an HA-, chondroitin- and chondroitin sulfate-binding domain is contacted with the mixture. If at least one of HA, chondroitin and chondroitin sulfate is present in the sample and bound to the immobilized HARE protein, peptide fragment or variant thereof, the labeled HARE protein, peptide fragment or variant thereof will bind thereto, and therefore can be detected by the presence of labeled HARE protein, peptide fragment or variant thereof on the immobilized HARE protein, peptide fragment or variant thereof.

In yet another embodiment, the present invention includes a method of treating an individual having an elevated level of at least one of HA, chondroitin and chondroitin sulfate in the blood or lymph by administering an effective amount of a vector encoding at least one of a functionally active HARE protein, a peptide fragment thereof, a variant thereof and a “HARE-like” protein. A “HARE-like” protein comprises a LINK domain and at least one motif selected from the group consisting of SEQ ID NOS:6-18 and sequences that are substantially identical to or only have conserved or semi-conserved amino acid substitutions to SEQ ID NOS:6-18, and is able to bind to and endocytose at least one of HA, chondroitin and chondroitin sulfate.

Other objects, features and advantages of the present invention will become apparent from the following detailed description when read in conjunction with the accompanying figures and appended claims.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1. Nucleic acid (SEQ ID NO:1) and deduced amino acid (SEQ ID NO:2) sequences of the 4.7-kb cDNA encoding the rat 175-kDa HARE. The artificial cDNA containing 4708 nucleotides encodes a 1431 amino acid recombinant 175-kDa HARE protein, whose deduced amino acid sequence begins with a serine. Amino acid sequences verified by peptide sequence analysis of the purified HARE are underlined, and the two N-terminal peptides found in the purified protein are underlined and in italics. Putative N-glycosylation sites are in boldface, and Cys residues are highlighted in boldface and italics. Three alternative N-glycosylation sites of the type -N-X-C- are located at N¹³⁵, N²¹⁸ and N⁹³⁰. The predicted transmembrane domain of the type I membrane protein is underlined and in boldface. The three shaded regions in the cytoplasmic domain are potential motifs for targeting the receptor to clathrin-coated pits. Potential HA-binding motifs of the type B-X₇-B, which are in the predicted extracellular domain, are enclosed in boldface [brackets].

FIG. 2. Nucleic acid (SEQ ID NO:3) and deduced protein (SEQ ID NO:4) sequences of the human 190 kDa HARE. The HARE nucleotide sequence was assembled based on the sequences of BAB15793 and specific RT-PCR products derived from human spleen (as described in detail previously in U.S. Ser. No. 09/842,930). The solid bars underline 17 consensus N-glycosylation sites. The arrow indicates a nucleotide sequence error in BAB15793 (omission of an A, in boldface) that results in a frame-shift, which adds 210 amino acids (in italics) and deletes eight at the N-terminal end of the ORF derived from BAB15793. A second error in the BAB15793 nucleotide sequence at T¹³⁸⁶ (rather than C) and noted in boldface is silent. Amino acid sequences within solid or dashed boxes indicate the peptides of the authentic human 190 kDa HARE (immunoaffinity purified from human spleen) that were identified, respectively, by direct sequencing or by molecular mass analysis (as described in detail previously in U.S. Ser. No. 09/842,930). Human spleen HARE amino acid sequences that were not in the BAB15793 protein sequence but were confirmed in peptide products are boxed and underlined.

FIG. 3. Domain structure of the 175 kDa rat HARE protein. The scheme depicts the organization of multiple protein domains within the 1431 amino acid HARE protein that are identified by numerous predictive search programs such as SMART, CD-Search, and other sites linked to ExPASy or NCBI. TM indicates the transmembrane domain; E2, Ea and Ec represent, respectively EGF-2, lamin-like EGF and EGF-Ca⁺² domains; potential N-linked glycosylation sites are indicated by the Y symbols.

FIG. 4A. Reactivity of a panel of 175HARE-mAbs in Western analysis after nonreducing SDS-PAGE of LEC extracts. Ascites from 11 hybridoma clones that were positive in ELISA screens with the 175HARE antigen were screened (at a 1:1,000 dilution) for reactivity with lysates of rat LECs. Seven of these clones showed strong reactivity with proteins at both 175 and 300 kDa (lanes 1-8 except lane 3). Clone 54 only recognizes the reduced protein (FIG. 4B). Three clones gave very different patterns (lanes 9-11) and do not recognize the 175HARE antigen. R and N show mouse antisera raised against reduced (R) or nonreduced (N) 175HARE antigen. The solid and open arrows indicate the positions of the 300HARE and 175HARE, respectively.

FIG. 4B. Reactivity of a panel of anti-175HARE mAbs in Western analysis after reducing SDS-PAGE of LEC extracts. Only mAbs 54 (lane 3) and 159 (lane 5) show strong reactivity which is identical with the reduced 175HARE and 300HARE proteins. The solid and open arrows indicate the positions of the nonreduced 300HARE and 175HARE, respectively. MAb-174, which also blocks HA binding (FIGS. 5 and 6), shows weaker reactivity with the reduced 175HARE and the 260 kDa subunit of the 300HARE (lane 6). The other mAbs, including those positive for the nonreduced proteins, are not reactive.

FIG. 5. Antibody inhibition of HA endocytosis by HARE in LECs. Cultured primary rat LECs were washed and incubated for 60 min at 37° C. with 2 μg/ml ¹²⁵I-HA in MEM medium containing 0-9 μg/ml of IgG (affinity purified from ascites fluid using Protein G-Sepharose, or rabbit anti-mouse IgM-Sepharose in the case of #159) from each of five different hybridomas against the 175HARE. The plates were then chilled on ice, the media was aspirated, the wells were washed 3 times and the cells were solubilized in 0.3 N NaOH. Radioactivity and protein content were determined for each of the samples. The mean of triplicates±SD are expressed as percent of control (dpm/mg protein).

FIG. 6. Specific monoclonal antibodies against HARE inhibit HA endocytosis in SK− Hep1 transfectants expressing the 175 kDa HARE. The indicated SK-Hep1 clones expressing the 175 kDa HARE were allowed to internalize ¹²⁵I-HA as described above with no addition or in the presence of either mAb-174 or mAb-235 as indicated. mAb concentration was 30 μg/ml.

FIG. 7. Immunocytochemical localization of HARE in human liver, spleen and lymph node. Sections of human spleen (A and B), lymph node (C) and liver (D) were treated with either anti-HARE mAb-30 (A, C and D) or mouse serum (B) and then stained. A relatively low magnification is shown (the bar represents ˜500 μm) to emphasize the localization of the human HARE protein in the sinusoidal regions of each tissue.

FIG. 8. Domain organization of the human 190 kDa HARE. The scheme depicts the organization of protein domains identified by the programs Pfam-HMM, CD-Search, ScanProsite or SMART (Schultz et al, (1998)). Abbreviations used for some of the domains include CD (cytoplasmic domain), TMD (transmembrane domain), M-T (metallothionein), and EGF-C, EGF-L or EGF-2 for epidermal growth factor calcium, laminin or type 2 domains, respectively.

FIG. 9. Sequence alignment of the human (SEQ ID NO:4) and rat (SEQ ID NO:2) HARE proteins. Sequences for the two smaller HARE proteins were aligned using SIM (at www. ExPASy, and as described in detail in U.S. Ser. No. 09/842,930) and then saved as a Microsoft Word file for highlighting and annotation. Identical residues found in both sequences are shaded in yellow. Conserved consensus N-linked glycosylation sides are in boldface and highlighted in gray. Solid black bars indicate potential -N-X-Cys- glycosylation sites, two of which are conserved. Cysteine residues are boldface and shaded red where identical between the two proteins. The arrow denotes the beginning of the least conserved regions of the two proteins: their cytoplasmic domains. The residues under the solid blue line are identified as an extracellular Link domain (XLink), a putative hyaluronan-binding domain. The residues under the dashed blue line indicate the single predicted transmembrane domain. The three conserved candidate φXXB motifs are within the two blue boxes. Ser, Thr or Tyr residues that are predicted (by NetPhos 2.0; Blom et al., (1999)) to be phosphorylated are shown in boldface white with red highlighting.

FIG. 10. Model for the organization of the two human spleen HARE isoreceptors. The 190 kDa and ˜315 kDa HARE isoreceptors isolated from human spleen are depicted as separate species in approximate molar ratios of 1:2, respectively. The 190 kDa HARE contains only one protein. The large HARE complex is composed of two (or perhaps three) disulfide-bonded subunits of about 250 kDa and one subunit of 220 kDa, respectively. Preliminary results indicate that the molar ratios of the affinity purified 190 kDa and ˜315 kDa HARE isoreceptors from different tissues may be different. All full-length HARE proteins and subunits are membrane-bound and are predicted to contain small cytoplasmic domains and very large ectodomains. The HARE proteins are elongated, rather than globular (Yannariello-Brown et al., (1997)).

FIG. 11. Scheme for HA turnover and metabolism in humans. The scheme depicts the overall turnover of HA present initially in the ECM of tissues throughout the body. Partially degraded HA is flushed from the ECM into lymph by the flow of fluid through the tissue. Some HA may be degraded locally in the tissue, but most HA (˜85%) is delivered to and removed by lymph nodes. The remaining HA (˜15%) enters the blood, and the majority thereof is cleared by the liver, while the spleen also removes a small fraction. HARE, which is expressed on the surface of sinusoidal endothelial cells of lymph node and liver, binds the circulating HA and removes it from the lymph or blood by internalization through the clathrin coated pit endocytic pathway. The average size and concentration of the HA decreases in going from ECM to lymph node to blood (Laurent and Fraser, (1992); Laurent and Fraser, (1991); Tengblad et al., (1986)).

FIG. 12. HARE is present in normal human bone marrow. Sections of normal human bone marrow were treated with either anti-HARE mAb-30 (upper panels and lower left panel) or mouse serum (lower right panel) and then stained.

FIG. 13. HARE is absent in a human bone marrow metastasis but is increased at the interface between cancer and normal marrow. Sections of human bone marrow metastasis were treated with either anti-HARE mAb-30 (upper right panel and lower panels) or mouse serum (upper left panel) and then stained. The tumor is to the upper left in all four panels.

FIG. 14. Carcinoma cells express cell surface HA. MDA-MB-231 (A) and PC3 (B) cells express cell surface HA as demonstrated by their staining with peroxidase following binding of a biotinylated HA binding protein. MDA-MB435 (C) and DU145 (D) cells show virtually no cell surface HA. This staining is specific for HA on the tumor cell surface, since it is virtually abolished (inserts) by pretreatment with the very specific hyaluronidase from Streptomyces.

FIG. 15. MDA-MB-231 and PC3 cells express a cell surface coat of HA. MDA-MB-231 (A) and PC3 (B) cells express cell surface HA coats as demonstrated by the particle exclusion assay. MDA-MB435 cells (C) or DU145 cells (not shown) show virtually no cell surface HA. This exclusion zone is due to HA on the tumor cell surface and is abolished by pretreating these cells with Streptomyces hyaluronidase (inserts).

FIG. 16. A mixed-cell aggregation assay to measure cell adhesion. SK-HARE cells expressing the rat small HARE isoform (or SK-Hep1 cells in control experiments) were labeled with orange fluorescent DilC₁₈ (1,1′-dioctadecyl-3,3,3′,3′-tetramethylindocarbocyanine perchlorate; from Molecular Probes) and PC3 (or other carcinoma) cells were labeled with green fluorescent calcein AM (Molecular Probes) for 40 min at 37° C. The cells were washed, chilled, released by mild trypsin treatment, collected by centrifugation, and resuspended in medium without serum. After incubation for 30 min at 37° C. to allow recovery of cell surface proteins, 10⁵ labeled SK-HARE cells were mixed with 10⁵ labeled PC3 cells and allowed to aggregate for 30 min at 37° C. with gentle mixing. The cells were chilled and mixed-cell aggregates (i.e. green and orange fluorescent aggregates) were assessed using epi-fluorescence microscopy at low magnification (100×). The number of aggregates was counted in each of 10 separate randomly chosen fields. In the figure, the three largest aggregates have both cell types, whereas many of the smallest “dots” (single cells) are only orange (e.g. around the “A” in left panel) or only green (e.g. in the upper right area of B) but not both colors.

FIG. 17. Cells expressing HARE adhere to cancer cells via HARE-HA interactions. The three different types of human cancer cells and SK-HARE cells were fluorescently labeled as in FIG. 16. Panel A: Calcein AM-labeled cancer cells were mixed with SK-HARE or SK-Hep-1 cells labeled with DilC₁₈ and then scored for the mean number of mixed aggregates/field as described in FIG. 16. Panel B: The experiment was performed as in A, except the SK-HARE cells were pre-incubated with 300 μg/ml of exogenous HA (44 kD), which was maintained throughout the aggregation assay, in order to occupy the endocytic HARE receptor. Panel C: The experiment was performed as in A, except the cancer cells were treated with 16 U/mL Streptomyces hyaluronidase for 1 hr and then mixed with SK-HARE cells to initiate aggregation; hyaluronidase was maintained throughout the assay period to minimize de novo HA synthesis by the tumor cells. Panel D: The experiment was performed as in A, except the SK-HARE cells were incubated with 5 μg/ml of the anti-HARE monoclonal antibody mAb-174 IgG for 30 min on ice prior to mixing with the cancer cells and performing the mixed-cell aggregation assay. Previous disclosures have shown that mAb-30 blocks HA binding to the rat HARE protein either in ligand blot assays, or in live cells.

FIG. 18. Human breast carcinoma metastasis to lymph node expresses cell surface HA and arrests at sites of HARE expression. Cases of breast ductal carcinoma were identified by computer search of the surgical pathology database at the University of Rochester following approval from the Institutional Research Subjects Review Board. The original hematoxylin and eosin stained sections were reviewed and tissue blocks of the primary breat carcinoma as well as a representative axillary lymph node were selected for study. The tissue was fixed in 10% neutral buffered formalin and paraffin embedded at the time of original surgery using routine methods. Sections (5 mm) were cut and allowed to dry overnight at 60° C. Paraffin was removed through a series of xylene and alcohol washes, and endogenous peroxidase activity was quenched with 3% hydrogen peroxide. The anti-HARE antibody mAb #30 and the preimmune serum required pepsin digestion for antigen retrieval. The slides were placed in a prewarmed solution (50 ml) of 0.3 mg/ml pepsin in 0.1N HCL and incubated at 37° C. for 15 min. The slides for biotin-HA binding protein required no antigen retrieval, however a hyaluronidase digestion was employed to assess specificity. The slides were washed with Phosphate Buffered Saline (PBS) containing (137 mM NaCl, 15 mM KCl, 1.4 mM sodium phosphate dibasic, 1.47 mM potassium phosphate monobasic, pH 7.4) and incubated with the appropriate primary antibody diluted in PBS at room temperature for 60 min. After the PBS washes, the slides were treated with secondary antibody conjugate (biotinylated horse anti-mouse, 1:200) for 30 min at room temperature. After the PBS washes, streptavidin peroxidase (1:1000) was then applied to the slides for 30 min at room temperature, the slides were then washed once with PBS and once with distilled water. Color development was for 5 min with 2.0% v/v aminoethylcarbazole and hydrogen peroxide according to the manufacturer's instructions (ScyTek, Utah). Hematoxylin was used for counterstaining. Slides were viewed with an Olympus BH-2 light microscope equipped with an Olympus 35 mm camera for photomicroscopy. Human metastatic breast carcinomas expressing cell surface HA was demonstrated by staining with biotinylated HA binding protein without (panel A) and with (panel B) hyaluronidase treatment. Arrest of metastatic cells in axillary lymph nodes (panel C) appears to occur at sites of HARE expression. Negative control using non-immune mouse serum is shown in panel D.

FIG. 19. Perfusion of isolated rat liver with ¹²⁵I-HA. The presence of unlabeled HA inhibits ¹²⁵I-HA clearance by intact liver. Rat livers were perfused ex vivo with recirculation medium containing 0.25 μg/ml ¹²⁵I-HA with no additions (●), or 50 μg/ml unlabeled HA (▪) as described in the Methods section. Each point is the mean±S.D. of duplicates from 34 perfused livers (n=6-8). The values are calculated as the percent of intact ¹²⁵I-HA remaining in the medium relative to the starting value.

FIG. 20. Perfusion of isolated rat liver with ¹²⁵I-HA. Rat livers were perfused ex vivo with recirculation medium containing 0.25 μg/ml ¹²⁵I-HA with 5 μg/ml mouse IgG (▴), or 5 μg/ml mAb-174 (●) as described in the Methods section. Each point is the mean±S.D. of duplicates from 3-4 perfused livers (n=6-8). The values are calculated as the percent of intact ¹²⁵I-HA remaining in the medium relative to the starting value. The anti-HARE blocking antibody mAB-174 specifically inhibits HA clearance by intact liver. Mouse IgG, used as a control, had essentially no effect on HA clearance (compare to “No addition” in FIG. 19).

FIG. 21. Perfusion of isolated rat liver with ¹²⁵I-HA. The anti-HARE blocking antibody mAb-174 specifically inhibits HA degradation by intact liver. Rat livers were perfused ex vivo with medium containing 0.25 μg/ml ¹²⁵I-HA with no additions (▪), 5 μg/ml mouse IgG (♦), 5 μg/ml mAb-174 (▾), 5 μg/ml mAb-30 (▴), or 50 μg/ml unlabeled HA (●), as described in the Methods section. Each point is the mean±S.D. of triplicates from 3-4 perfused livers (n=9-12). The values are calculated as the percent of the initial intact ¹²⁵I-HA at the beginning of the perfusion.

FIG. 22. Methods of targeting a compound to or preventing interaction with a cell expressing HARE. A mAb-drug conjugate, HA-drug, or HA/CS-mimetic-drug may be utilized for delivery of cancer drugs to liver, lymph node, spleen and/or bone marrow (major sites of metastasis). A blocking monoclonal antibody or an HA/CS-mimetic may be utilized in (1) a method of blocking the process of metastasis in which cancer cells naturally coated with HA target to liver, lymph node, spleen and/or bone marrow by interaction with HARE on sinusoidal endothelial cells, or (2) a method of blocking the unwanted uptake and clearance (by liver, lymph node, spleen and/or bone marrow) of HA-drug or CS-drug conjugates. In this second situation, the HA/CS in the drug conjugate is intended to either (i) target and interact with other HA receptors in a particular tissue or cell type, such as but not limited to, CD44, for anti-cancer applications, or (ii) stabilize, protect or increase the useful half-life of the drug. In addition, an extracellular HA-binding domain of HARE (or an extracellular chondroitin-binding domain or an extracellular chondroitin sulfate-binding domain) may be utilized for a clinical ELISA test kit for the quantitation of HA (or chondroitin or chondroitin sulfate) in biological fluids. In addition, an HA-binding domain, a chondroitin-binding domain, or a chondroitin sulfate-binding domain of HARE may be utilized in a solid phase material for the removal of HA, chondroitin and/or chondroitin sulfate from the blood of patients on dialysis. In addition, a defined GAG sequence (such as but not limited to a CS-A 8 mer) as a substitute for the mimetic described herein above.

FIG. 23. Comparison of HA binding by the native and recombinant 175-kDa HARE proteins. Membranes from isolated LECs (lanes 1 and 2) and SK-175HARE-34 cells (lanes 3 and 4) were solubilized in TBS containing 0.5% NP40 plus protease inhibitors, and HARE proteins were immunoprecipitated using mAb-30 coupled to Sepharose. The proteins were eluted with sample buffer, subjected to SDS-PAGE and electrotransfer, and the nitrocellulose was incubated overnight in TBS containing 0.5% Tween-20. Ligand blotting with 1 mg/ml ¹²⁵I-HA (lanes 1 and 3 from autoradiogram) was performed as described previously in U.S. Ser. No. 09/842,930. The same blots were then incubated in TBS containing 1% BSA and subjected to Western analysis (lanes 2 and 4) using a mixture of eight mAbs against HARE. A series of dilutions verified that the Western staining responses for both samples were proportional to protein load and were not saturated. The open and solid arrows indicate, respectively, the ˜300-kDa and 175-kDa HARE species. The HA-binding intensity relative to the Western staining of the 175-kDa HARE was essentially the same from LECs and the stable cells.

FIG. 24. Cell surface expression of the recombinant 175-kDa HARE in stably transfected cells. After blocking nonspecific binding sites, SK-175HARE cells or SK-Hep-1 cells transfected with vector alone were incubated, as indicated, with either nothing, 1 mg/ml mAb-30, 1 mg/ml mouse IgG or a mixture of four mAbs (#s 30, 154, 174 and 235 each at 1 mg/ml). The cells were washed, incubated with Alexa 488-conjugated secondary antibody for 45 min on ice and processed for FACS analysis.

FIG. 25. FACS analysis of fl-HA uptake in SK-175HARE cells mediated by the 175-kDa HARE. SK-Hep-1 cells transfected with vector alone (panel A) or SK-175HARE-34 cells (panels B and C) were grown to confluence in 6-well tissue culture plates, washed and preincubated at 37° C., as indicated in the figure, with no addition or nonlabeled HA (panel B) or mouse IgG or mAb-174 (panel C) followed by fl-HA. The same five conditions were used in panel A.

FIG. 26. Transiently transfected 293 Flp-In cells express the HA-binding recombinant 190 kDa hHARE protein. 293 Flp-In cells (5×10⁴ per well) grown in 24-well plates were transfected with pSecTag-190 kDa hHARE/ExGEN500 or pSecTag/ExGEN500 complexes and allowed to recover for 2 days. The transfected cells were allowed to bind and endocytose 1 μg/ml ¹²⁵I-HA with (H+ C) or without (H) 100 μg/ml unlabeled HA in serum-free medium for 3 hours at 37° C. Cells were washed with ice-cold HBSS, solubilized in 0.3 M NaOH, and radioactivity and protein were determined.

FIG. 27. The 190 kDa hHARE is expressed abundantly and is biologically functional in stably transfected 293 Flp-In cells. A, Whole cell lysates from three clones expressing the 190 hHARE (#9, #14, #40) and one clone transfected with empty vector (EV) were subjected to nonreducing SDS-PAGE using a 5% gel, followed by electrotransfer to nitrocellulose. Bottom Panels: After blocking in 0.1% Tween-20 in TBS for 3 hr at room temperature, ligand blotting and autoradiography (AR) were performed, using 1.0 μg/ml ¹²⁵I-HA with (left) or without (right) 100 μg/ml unlabeled HA, as described under “Methods”. Top Panels: The same nitrocellulose strips were then rewet, blocked with 1% BSA in TBS and Western Blot (WB) analysis was performed using a mixture of mAb30, mAb154, and mAb159. B, Whole cell lysates from clone #9 were subjected to nonreducing (NR) or reducing (R) SDS-PAGE using a 5% gel and electrotransfer to nitrocellulose. After blocking for 2 hr in 1% BSA in TBS, the nitrocellulose was cut into strips and subjected to Western Blot analysis with 1 μg/ml of the indicated seven mAbs previously raised against the rat 175 kDa HARE. C, Cells expressing the 190 hHARE protein were lysed in Laemmli buffer and either treated with Endoglycosidase F and/or reduced with 10 mM dithiothreitol (DTT). Proteins were separated on a 5% SDS-PAGE. Bottom Panel: The transfer was incubated with 1 μg/ml ¹²⁵I-HA with (left) or without (right) 100 μg/ml unlabeled HA for 2 hr at 4° C., washed and autoradiography was performed. Top Panel: The nitrocellulose was then rewet in 1% BSA in TBS and Western Blot analysis was performed to identify hHARE using anti-V5 antibody.

FIG. 28. Kinetics of ¹²⁵I-HA binding by stable cell lines expressing the 190 kDa hHARE. Confluent cell cultures of hHARE expression clones #9 (◯) and #14 (▪) were incubated at 37° C. in medium without serum for 60 min. The plates were placed on ice and the wells were washed once with HBSS. The cells were permeabilized at 4° C. with 0.55% digitonin in PBS to allow access to both surface and internal receptors (Weigel et al., 1983; and Oka and Weigel, 1983). The cells were washed, and then incubated in medium containing 1.5 μg/ml ¹²⁵I-HA with or without 150 μg/ml unlabeled HA. At the noted times, the cells were washed 3 times with HBSS, solubilized in 0.3 N NaOH, and protein and radioactivity were determined as in “Methods”. Data shown represent specific binding; each point is the average of duplicate wells without excess HA (total binding) minus the average of duplicate wells with excess HA (nonspecific binding).

FIG. 29. The recombinant 190 hHARE mediates continuous endocytosis and degradation of ¹²⁵I-HA. A, Confluent cell cultures of 190hHARE-expressing clones #9 (●) and #14 (▪) and empty vector control clone #13 (▾), in 24-well tissue culture plates, were incubated at 37° C. in medium without serum for 30 min. The plates were then placed on ice, and the cells were washed once with HBSS. Medium containing 1.6 μg/ml ¹²⁵I-HA with or without 160 μg/ml unlabeled HA was added to each well, and the cells were incubated at 37° C. for up to 4 h to allow internalization. At the noted times, the medium was removed, and the cells were washed three times with 1 ml of HBSS, lysed in 0.3N NaOH, and cell protein content and cell-associated radioactivity were determined. The data are shown as specific uptake; the average radioactivity values of duplicates for total uptake minus the average of duplicates for nonspecific uptake. B, Clone #14, expressing the 190 hHARE, was cultured in 4-well tissue culture plates, and processed as in A. In addition, degraded ¹²⁵I-HA that was cell-associated and in the medium were also measured at the noted times, as described in “Methods”. The plots show radioactivity (representing intact and degraded HA) associated with the cells (▪) and the total degraded ¹²⁵I-HA (●), i.e. products still inside the cell plus those in the medium.

FIG. 30. Kinetic and Scatchard analyses of ¹²⁵I-HA binding to Flp-In 293 cells expressing recombinant 190 kDa hHARE. 190hHARE cell lines#9 (▪) and #14 (●) were cultured in 4- or 6-well plates until confluent. The cells were incubated for 60 min at 37° C. in medium without serum and then chilled to 4° C. for all subsequent steps. The cells were washed with HBSS, permeabilized with 0.055% digitonin for 15 min and then washed with HBSS. At the end of each experiment, cells were washed with HBSS and cell-associated radioactivity and cell protein were determined as described in Methods. Panel A, After washing the cells, medium containing 0.1 μg/ml ¹²⁵I-HA with the indicated amount of unlabeled HA was added to each well and the cells were allowed to bind the HA on ice for 90 min. Specific binding was ˜90%, as assessed in the presence of the highest HA concentration. Panel B, The data in A for clones #9 and #14 were recalculated as specific HA binding (femtomoles of HA/10⁶ cells). The data are the mean±SD of duplicates for each of the two clones (n=4). Panel C, After the cells were allowed to bind HA on ice for 90 min as in A, the medium was removed to determine free ¹²⁵I-HA, and the cells were washed and cell-associated ¹²⁵I-HA was determined. The specifically bound HA was calculated for the experiment shown in B and the results are presented in the format of Scatchard (Scatchard, 1949) as the average of duplicates for each cell line.

FIG. 31. Only some non-HA GAGs compete for ¹²⁵I-HA endocytosis at 37° C. by stable cell lines expressing the recombinant 190 kDa hHARE. Cells from 190 hHARE Flp-In 293 clones #9 and #14 were incubated at 37° C. for 3 h in medium containing 1.5 μg/ml ¹²⁵I-HA with 3 to 100 μg/ml of the indicated GAG. The values for competition of ¹²⁵I-HA internalization by unlabeled GAGs or HA (expressed as a percent of the no-competitor control) are the average of duplicates from the two clones (n=4). A: keratan sulfate (▴), dermatan sulfate (♦), chondroitin (▪), chondroitin sulfate A (▾), HA (●). B: Heparan sulfate (⋄), heparin (∇), chondroitin sulfate E (Δ), chondroitin sulfate D (◯), chondroitin C (□), HA (●).

FIG. 32. Chondroitin sulfates and other GAGs do not compete well for ¹²⁵I-HA binding at 4° C. by Flp-In 293 cells expressing the 190 hHARE. After a serum-free incubation and wash with HBSS, cells from 190 hHARE clones #9 and #14 were incubated at 4° C. for 2 h in medium containing 1.5 μg/ml ¹²⁵I-HA and 50 μg/ml of the indicated GAG. The values for each GAG are the mean of duplicate samples from both clones (n=4)±the standard error.

FIG. 33. Other GAGs compete for ¹²⁵I-HA binding to recombinant 190 hHARE in a ligand blot assay. Extracts prepared from 190hHARE clone #14 cells were subjected to SDS-PAGE and electroblotted as described in “Methods”. The nitrocellulose was blocked with TBS and 0.1% Tween 20 at room temperature for 3 h and 3 mm strips were cut and placed in Buffer 1 with 0.5% sodium azide, 5 mM EDTA, 0.05 μg/ml ¹²⁵I-HA and 50 μg/ml of the indicated GAG or HA. The strips were incubated at 4° C. for 2 hr on a rocking platform. The medium was then removed, the strips were washed extensively with TBST for 20 min, allowed to air dry, and put down for autoradiography with BioMax MS film. The exposure shown was for 19.5 h at −85° C. with 2 intensifying screens. The graph shows the average densitometry values±SE from 3 separate samples for each GAG.

FIG. 34. The human and rat small HARE isoforms show different GAG specificities for competition of ¹²⁵I-HA endocytosis. Flp-In 293 190hHARE clones #9 and #14 (white bars), SK-HARE clones #26 and #35 (expressing recombinant rat 175 kDa HARE), and liver sinusoidal endothelial cells (from freshly perfused rat liver) were incubated at 37° C. for 3 h with medium containing either 1 μg/ml ¹²⁵I-HA and 30 μg/ml of the indicated GAG (for SK-HARE and liver cells) or 1.5 μg/ml ¹²⁵I-HA and 100 μg/ml of the indicated GAG (for 190hHARE 293 cells). Cells were then washed, lysed and the radioactivity and protein content were determined as described in “Methods”. Each GAG value is the mean±SE of at least 4 individual wells (e.g. two wells for each of two clones or LEC preparations) and is calculated as a percentage of the ¹²⁵I-HA control (without competitor).

FIG. 35. Inhibition by anti-HARE mAbs of ¹²⁵I-HA uptake by cells expressing recombinant 190 hHARE. Flp-In 293 190hHARE clones #9 and #14 were cultured and processed as described in FIG. 29. Panel A, The cells were allowed to bind and endocytose 1.5 μg/ml of ¹²⁵I-HA for 3 h at 37° C. with no additions, or the noted concentration of anti-rat HARE mAbs 28 (●), 30 (▪), 154 (▴), 159 (▾), 174 (♦), 235 (◯), and 467 (□). The cells were processed as described in “Methods”. The values shown are the average of two replicate wells from each clone (n=4), expressed as a percent of the no-addition control specific binding values. Mouse IgG (not shown) at 10 μg/ml was 103±5% of the control value. Specific binding (CPM/μg cell protein), as assessed in the presence of a 100-fold excess of unlabeled HA, was 87%. Panel B, The indicated purified antibodies were used singly or in combination, at concentrations of 20 μg/ml, in an experiment performed as described in A. Nonspecific endocytosis was assessed in the presence of 75 μg/ml unlabeled HA.

FIG. 36. Some anti-HARE monoclonal antibodies inhibit HA endocytosis by mouse LECs. Mouse LECs were isolated by a collagenase liver perfusion procedure followed by differential centrifugation and purification over Percoll gradients. Cells were plated on fibronectin-coated 24-well plates in RPMI medium without serum and used the same day. Cells were incubated at 37° C. for 3 hours with medium containing 1.5 μg/ml ¹²⁵I-HA and either 150 μg/ml unlabeled HA or 50 μg/ml of the indicated monoclonal antibody (mAb; raised against the rat 175 kDa HARE). Panel A, The results for three separate experiments are shown as the average of duplicates: black bars, experiment 1; white bars with hatch pattern, experiment 2 (note that mAbs 28 and 467 were not used in this experiment); gray bars, experiment 3. Panel B, Values for specific ¹²⁵I-HA uptake were calculated by subtracting the nonspecific uptake, determined in the presence of a large excess of unlabeled HA as in A. Uptake values in units of CPM/μg protein were normalized to the no-addition control (set at 100%) and are presented as the mean±SEM for samples from experiments 1 and 2 (the black and hatched bars), each determined in duplicate (n=4), except for mAb-28 and mAb-467 which were duplicates (n=2) from experiment 1 only.

FIG. 37. Identification of some HARE splice variant cDNAs in human spleen. Splice variant transcripts of the human HARE (hHARE) gene (Stab2) were amplified from a human spleen cDNA pool (Marathon, Clontech) using five different primer sets (numbered 1-5) spanning nucleotide 3405 through 7656. Nucleotide numbering is based on the hypothetical full-length (7656 nucleotides) complete ORF of the HARE/Stabilin 2 gene product. Panel A: The first round of amplification was performed with each of the five primer sets (as indicated) using the human spleen cDNA (first lane of each pair) or a synthetic control cDNA containing the complete 190 kDa hHARE sequence (second lane of each pair). With each primer pair, only one major band was apparent (except as noted below in B), which was identical to the band amplified from the control and corresponded to the expected full-length PCR product. Panel B: The scheme shows the various domains of the 190 kDa hHARE protein and the regions of the corresponding cDNA sequence amplified by the five Primer pair sets. T, transmembrane domain; CD, cytoplasmic domain; Link, Link domain; Cys-rich, cysteine-rich domains 3 and 4; black boxes are intervening domains. Panel C: Undetectable shorter DNA products that might be present were purified from the excised regions of the gels shown on the left (the areas within the white boxes immediately below each of the amplified product bands produced during the first round of PCR in A). Any DNA present in these samples was subjected to a second round of PCR amplification using the same set of primer pairs (shown on the right). Based on the appearance of new discrete bands, three of the five primer pair sets (numbered 2, 4, and 5) resulted in the amplification of rare transcripts in this 2-step PCR procedure. Primer pair set 2 amplified nucleotides 3673-4890 (exons 35-46); primer pair set 4 amplified nucleotides 5491-6621 (exons 53-60); primer pair set 5 amplified nucleotides 6595-7656 (exons 61-69).

FIG. 38. Identification of HARE splice variants in human spleen after one round of PCR with primer pair #5. The products from one round of PCR using primer pair #5 (as in FIG. 37) with spleen (lane 2) and lymph node (lane 3) cDNA pools were subjected to agarose gel electrophoresis. DNA markers and their base-pair (bp) size are shown in the lane 1 (M). The broad band in lanes 2 and 3 is from wt HARE and the minor band (arrow) in the spleen lane is an amplified splice variant, which is missing the 108 bp exon 63. As noted in Table V, this variant is called: hHAREv(62/64)fs.

FIG. 39. Splice variants of human HARE are also present in lymph node. Splice variant transcripts of the human HARE (hHARE) gene (Stab2) were amplified from a human lymph node cDNA pool (Marathon, Clontech) using five different primer sets (numbered 1-5, as noted in FIG. 37). After electrophoresis of the PCR mixtures on a 1% agarose gel and staining with ethidium bromide, images were captured digitally using an Alpha Innotech FluoroChem Model, version 2. The inverse (white for black and black for white) of the original image is shown. M=lane with kb marker ladder. Panel A: The first round of amplification was performed with each of the five primer sets (lanes 1, 2 with set #1; lanes 3, 4 with set #2; lanes 5, 6 with set #3; lanes 7, 8 with set #4; lanes 9, 10 with set #5;) using the human lymph node cDNA (lanes 1, 3, 5, 8, and 9) or a synthetic control cDNA containing the complete 190 kDa hHARE sequence (lanes 2, 4, 6, 7, and 10). Lane 11 shows a positive control for the cDNA pool using a primer pair designed by the Manufacturer to amplify cDNA from part of the glyceraldehyde-3-phosphate dehydrogenase gene. With each primer pair, only one major band was apparent, which was identical to the band amplified from the control and corresponded to the expected full-length PCR product. Panel B: Undetectable shorter DNA products that might be present were purified from the excised regions (highlighted by the dashed black or solid white boxes) of the lymph node lanes, for each of the primer pair sets shown in Panel A. Any DNA present in these samples was subjected to a second round of PCR amplification (as in FIG. 37) using the same set of primer pairs: lane numbers 1, 2 and 3 correspond to the boxes in lanes 1, 3 and 5, respectively, in A; lane 4 is from lane A8 (white box, which contained a faintly visible band); lane 5 is from lane A8 (black box); and lane 6 is from lane A9. Each visible PCR product band was excised and sequenced. In each case, three types of PCR products are expected: the original major full-length product (e.g. the major band in lane A1), the hHARE variants being sought and undesired (irrelevant) nonspecific products (nonhHARE transcripts, presumably amplified in a nonspecific manner, e.g. the two smaller minor bands in lane 3). The major band in lane 2 (˜0.6 kb, marked with an asteric) is a hHARE variant with a large portion of Cys-rich Region #3 deleted and the resulting splice is in-frame. This band also contains a minor fraction of sequences with a 5-base deletion at position 3843-3847. The band at ˜1.1 kb in lane 2 corresponds to the full-length control fragment. The band in lane 4 marked with a black dot is the same as the spleen variant hHAREv(58/61) noted in FIG. 40. The bands just below and just above this band are, respectively, a nonspecific PCR product derived from a calcium channel and the full-length control fragment.

FIG. 40. Sequences of initial HARE splice variant cDNAs found in human spleen. The individual PCR amplification products shown in FIG. 37C were excised from the gel, gene-cleaned and sequenced directly. The cDNA coding sequence corresponding to these initial splice variants is shown. For each excised band, the top two lines of text indicate the native nucleotide sequence with the corresponding amino acid sequence just below it. The following two lines are the nucleotide and predicted amino acid sequences of the splice variant. Exon nucleotide sequences on either end of the altered exon regions are shown in boldface font. Numbers above the nucleotide sequence are the exon numbers in the human HARE (Stabilin 2) gene. The following terminology summarizes the results found: hHAREv(62/64fs) indicates a variant in which exon 63 is excised with a resulting frame-shift occurring in exon 64, hHAREv(37/39fs) indicates that exon 38 is excised with a frame-shift occurring in exon 39; hHAREv(58/61) means that exons 59 and 60 are excised and the coding region of the resulting transcript is in-frame; hHAREv(˜62/˜67) means that exons 63-66, as well as a portion of exons 62 and 67, are excised and the resulting transcript is still in-frame. For those splice variants in which a frame shift occurs, the underlined letter indicates a nucleotide at a splice junction that is either retained (e.g. HARE62/64fs) or lost (e.g. HARE37/39fs) and that results in the frame-shift. For hHAREv(˜62/67), the excision of the coding sequence does not occur at the exon boundaries; rather this variant junction is between sites that are 10 nucleotides upstream from the 5′ end of exon 62 and 90 nucleotides downstream of the 3′ end of exon 67. The boldface and italicized region indicates the nucleotides that are lost from coding exons 62 and 67.

FIG. 41. Exon organization of the full-length hHARE gene and the coding exons represented in some spleen and lymph node splice variant transcripts. The hHARE (Stab-2) gene has 69 exons depicted by the rectangles. Red exons represent four Cys-rich domains, which contain multiple fascilin and EGF-like domains. The green exon encodes a LINK domain, found entirely within exon 61. The blue exon and the maroon exons encode the transmembrane (TM) and cytoplasmic domains (CD), respectively. The first four splice variants contain the complete ORF of exon #1, approximately half of which encodes the signal sequence. The last 5 splice variants were identified by the method outlined in FIG. 37. The arrows indicate regions that are currently being sequenced to determine if the remaining regions are identical to wt HARE or if they are missing any additional exons. The yellow regions represent frame-shifted variants (fs) caused by a splice occurring within a codon that creates additional unique amino acid sequence at the C-terminal end of the putative protein. Variants that lack the blue TM domain would be translated as soluble proteins that are secreted.

FIG. 42. Recombinant hHARE splice variants or artificial deletants are expressed in mammalian cells. A. Transient expression of 190-hHARE deletion constructs (“designed variants”, not splice variants) in Flp-In 293 cells. Cells (2-days post transfection) expressing different N-terminal deletion mutants of 190hHARE were collected and lysed in Laemmli buffer. Samples were reduced with DTT, alkylated with iodoacetamide and proteins were separated by SDS-PAGE (8% gel). After electrotransfer to nitrocellulose, the 190-hHARE variants were detected with anti-V5 antibodies. Each variant, represented by the predominant band in each lane, migrated slightly faster (not shown) when the samples were not reduced, indicating that the proteins are likely to be folded correctly. Vector alone controls were completely negative. Lanes correspond to hHARE variants with deletions of the indicated amino acids: 1=(Δ1-1063); 2=(Δ1-695), 3=(Δ1-485), 4=(Δ1-89). B. Transient expression of 190-hHARE splice variants in Flp-In 293 cells. Expression vectors containing cDNAs for wt 315 kDa hHARE (lane 4), no cDNA (mock transfection; lane 3) and three different splice variants from spleen (lane 2 [v13/69])) or lymph node (lane 1 [v1/64] and lane 5 [v35/66]) were transiently transfected into Flp-In 293 cells. Two days later the cells and media were harvested, lysed and immunopurified using resin containing anti-V5 epitope antibody as described in methods. Proteins were eluted from the resin and subjected to SDS-PAGE and electrotransfer to nitrocellulose. Blots were developed with rabbit anti-V5 polyclonal antibody, followed by goat anti-rabbit-alkaline phosphatase conjugate and development with p-nitro blue tetrazolium and sodium 5-bromo-4-chloro-3-indolyl phosphate p-toluidine. The expressed hHARE splice variants in lanes 1, 2 and 5 are indicated by arrows (along with the wt protein in lane 4) and demonstrated molecular masses of, respectively, 55.8, 74.3 and 174 kDa.

FIG. 43. HARE is expressed in rat liver in a cyclic manner during embryonic development. HARE is highly expressed in rat embryonic liver and is detected as early as day-12/13 (A). No staining was seen in other tissues except for the amnion membrane at day-10; but not at day-11 (B). An intriguing feature of HARE expression in liver is that it is up-regulated, down-regulated and then up-regulated again in going from day-13 to day-18 (A). HARE expression is evident at day-13 and very high on day-15 (left panel, middle row), becomes very low then absent on day-17 (left panel, bottom row), but then is very high again on day-18 (right panel, bottom row). Slides were obtained from MTR Scientific Products and immuno-histochemistry was performed using anti-HARE mAb-30. Controls with non-immune mouse IgG showed no staining and appeared the same as the Day-17 sample (left panel, bottom row). The novel HARE expression pattern in developing liver is likely due to fetal-specific splice variant of HARE expressed during the day-13 to day-15 period. The adult form of HARE is likely expressed from day-18.

FIG. 44. Biotin-HA binding to purified recombinant s190 hHARE is dependent on concentration, time, and temperature. Polysorb 96-well plates were treated with purified s190 kDa HARE (2.6 pmol/well) for 2 hr at room temperature, followed by blocking with 2% BSA in TBST. Biotin-HA was added as indicated, and the plates were incubated at either 37° C. (closed symbols) or 4° C. (open symbols). All wells were washed 3-times with TBST and bound HA was detected in a typical ELISA format using a streptavidin-alkaline phosphatase conjugate in the presence of substrate at 37° C. for 1 hr (●, ◯), 2 hrs (▪, □), or 2.5 hrs (▴, Δ). Results are the average of duplicates. Like the purified native rat or human HARE proteins, the recombinant sHARE binds HA at 37° C. in a dose-dependent manner, but binds little or no HA at 4° C.

FIG. 45. The purified recombinant s190 kDa and s315 kDa hHARE ectodomains bind biotin-HA. Equal amounts (μg) of the purified soluble hHARE proteins were adsorbed onto Polysorb ELISA wells, and incubated with increasing concentrations of biotin-HA as described in FIG. 44. Detection was carried out using a streptavidin-alkaline phosphatase conjugate in the presence of substrate for 2 hr at 37° C. The A405 values in each well were normalized to the molar amount of each protein. The solid line was calculated by second order regression analysis using all data for both the s190 (●) and the s315 (◯). The dashed lines denote the 95% confidence intervals. The results demonstrate that both proteins bind HA with similar kinetics and to the same extent.

FIG. 46. Some but not all GAGs bind to the recombinant s190 kDa hHARE ectodomain. ELISA like assays were performed as described in Methods and in FIGS. 44 and 45 using 2.6 pmol of purified s190 hHARE protein adsorbed to each well. Two different concentrations (0.5 and 1.0 μM, as indicated) were tested for each of the ten biotin-GAGs.

FIG. 47. Dose response of biotin-CS-D binding to increasing amounts of s190 hHARE protein. Increasing amounts of purified s190 hHARE protein was adsorbed to the ELISA wells, as indicated. The wells were washed, incubated with 400 nM biotin-CS-D, and processed to determine the amount of CS-D binding as described in Methods and in FIGS. 44 and 45. Values are presented as the mean±SD (n=3).

FIG. 48. Biotin-CS-D binding to the s190 hHARE protein. The dose response for the binding of biotin-CS-D was determined as described in Methods and in FIGS. 44 and 45 using a fixed amount of purified s190 hHARE protein (2.6 pmol) adsorbed to each ELISA well and increasing concentrations of biotin-CS-D as indicated. Values are presented as the mean±SD (n=3).

FIG. 49. CS-E does not effectively block CS-D binding to the recombinant s190 hHARE protein. The binding of biotin-CS-D to s190 hHARE (2.6 pmol per well) was assessed as described in Methods and in FIGS. 44, 45 and 48 in the presence of no competitor (the 100% value) or the indicated amounts of unlabeled CS-A, CS-B, CS-D, CS-E, heparin or HA.

FIG. 50. The binding of biotin-CS-D to s190 hHARE is only partially competed by CS-E. Biotin-CS-D (400 nM) was allowed to bind to adsorbed purified s190 hHARE protein (2.6 pmol per well) as described in Methods and in FIG. 49 in the presence of no competitor (the 100% value) or increasing amounts of unlabeled CS-E as indicated. The values are presented as the mean±SE (n=3).

FIG. 51. Biotin-CS-D binding to s190 hHARE is competed by CS-D and CS-B but not KS. Biotin-CS-D (400 nM) was allowed to bind to adsorbed purified s190 hHARE protein (2.6 pmol per well) as described in Methods and in FIGS. 44 and 45 in the presence of no competitor (the 100% value) or increasing amounts of either unlabeled KS, CS-B or CS-D as indicated. The values for KS are presented as the mean±SE (n=3). The average of duplicates is shown for the other two GAGs.

FIG. 52. Nucleic acid (SEQ ID NO:95) and amino acid (SEQ ID NO:96) sequences of the full-length human HARE/Stab2 cDNA. FIG. 52A illustrates the nucleic acid coding sequence and exons (alternating boldface and normal font) of the full-length human HARE/Stab2 cDNA. This sequence differs from database sequence NM 017564 submission by three nucleotides at positions #3827 (A in NM, C in HARE; results in Asp [NM] or Ala [HARE]), #5811 (C in NM, T in HARE; results in no amino acid change [silent]), and #6537 (G in NM, A in HARE; results in no amino acid change [silent]). FIG. 52B illustrates the amino acid sequence of human HARE (Stabilin 2) precursor protein.

FIG. 53. Two active isoforms of human HARE are generated in cells expressing the full-length 315 kD hHARE cDNA. Stable Flp-In 293 cell lines were isolated after transfection with a vector containing the full-length human HARE cDNA and selection with Hygromycin B. Detergent lysates from several stable cell clones expressing HARE were pooled (lanes 2 and 4) and the HARE proteins were immunoprecipiated using a mixture of three mAbs coupled to Sepharose 4B (mAbs 30, 154 and 159 which recognize hHARE). The control lysate (lanes 1 and 3) was prepared from cells transiently transfected with vector lacking the hHARE cDNA insert. Adsorbed proteins were eluted with buffer containing SDS, separated by SDS-PAGE using a 5% gel and then electrotransferred to a nitrocellulose membrane. A ligand blot assay was performed using ¹²⁵I-HA followed by autoradiography (left panel). The same membrane was then subjected to Western Analysis (right panel) using rabbit anti-V5 antibody to detect the epitope tag on recombinant HARE proteins. The two HARE proteins apparent in lane 4 were both active, i.e. able to bind HA, and correspond to the previously identified native hHARE 190 kDa and 315 kDa isoforms. The results indicate that the smaller hHARE isoform is derived from a larger precursor produced from the full-length protein.

FIG. 54. The recombinant full-length 315 kD hHARE is active when expressed in human 293 cells. Flp-In 293 cells were transfected with the expression vector, described in Methods, containing cDNA encoding the recombinant full-length hHARE protein and stable cell lines were selected and screened as described for the 190 kD hHARE cell lines. Cells from the indicated four independent clones were grown in DMEM with 8% fetal calf serum, and processed as described in FIGS. 28 and 29. The cells were then incubated at 37° C. with medium (minus serum) containing 1.5 μg/ml ¹²⁵I-HA with or without a 100-fold excess of unlabeled HA. The latter nonspecific uptake values were subtracted from the values without excess HA to obtain specific HA values. At various times cultures were washed, cells were lysed, and radioactivity and protein were determined as described in Methods. Specific total cell associated HA (intact and degraded is shown in panel A, and degraded HA is shown in panel B.

FIG. 55. The small splice variant hHAREv(13/69) binds HA. Cells stably expressing variant 13/69 were generated, selected and grown as described in Methods. One ml each of conditioned medium from nontransfected Flp-In 293 cells, from cells stably expressing hHARE variant 13/69, and from cells stably expressing s190 hHARE were incubated for 2 hr at room temperature with either 5 μl or 10 μl of resin containing 0.1 mg/ml anti-V5 antibody. The resin was centrifuged and washed once with Tris-buffered saline containing 0.1% Tween-20 (TBST) followed by another incubation with 4 μg/ml biotin-HA for 1 hr at room temperature. The resin was washed 4-times with TBST and incubated for 30 min with Streptavidin-AP conjugate (0.1 μg/ml). The resin was then washed 6-times with TBST and incubated with 0.5 ml of p-nitrophenylphosphate according to the manufacturer's instructions. At 30 min (s190 hHARE samples) or 2 hr (13/69 hHARE samples), the resin was mixed by vortexing and centrifuged to pellet the resin. A volume of 150 μl for each sample was placed in a 96 well plate and the A₄₀₅ values were determined using an ELISA plate reader. The average of duplicates is shown.

### DETAILED DESCRIPTION OF THE INVENTION

Before explaining at least one embodiment of the invention in detail, it is to be understood that the invention is not limited in its application to the details of construction and the arrangement of the components or steps or methodologies set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

The term “functionally active HARE” as used herein will be understood to include a protein or peptide which is able to specifically bind at least one of HA, chondroitin and chondroitin sulfate, and when present on a surface of a cell, is able to endocytose the bound HA, chondroitin or chondroitin sulfate. The terms “functionally active fragment of HARE” and “functionally active variant of HARE” as used herein will be understood to include polypeptides which are able to specifically bind at least one of HA, chondroitin and chondroitin sulfate. Such active fragments or variants of HARE may include soluble fragments or variants of HARE. One of ordinary skill in the art, given this Specification containing descriptions of the cytoplasmic, transmembrane and extracellular domains of HARE and various variants of HARE that are soluble (as discussed in more detail herein below in the Example), should be able to identify and select portions of the HARE protein (e.g., the extracellular domain of HARE or portions thereof, such as an HA-binding domain of HARE) which retain the ability to bind at least one of HA, chondroitin and chondroitin sulfate.

In addition, the present invention also includes “HARE-like” proteins that are able to specifically bind at least one of HA, chondroitin and chondroitin sulfate. When the “HARE-like” proteins are present on a surface of a cell, the “HARE-like proteins” may further be able to endocytose the bound HA, chrondroitin and/or chondroitin sulfate. Such “HARE-like” proteins contain a LINK domain (as discussed in further detail herein after) and at least one other motif as defined in Table III.

The term “variant” as used herein will be understood to refer to refer to something which differs in form only slightly from something else, though the two are really the same. The terms “HARE variant” and “variant of HARE” as used herein will be understood to refer to proteins and polypeptides that vary from SEQ ID NOS:2, 4, or 96 and are able to specifically bind at least one of HA, chondroitin and chondroitin sulfate. Such proteins or polypeptides may include soluble variants of HARE. The term “HARE variant” as used herein will be understood to include naturally-occurring splice variants of HARE as well as designed variants of HARE. Naturally-occurring splice variants of HARE include HARE variants designed by nature, while designed variants of HARE include HARE variants designed by the hand of man. One of ordinary skill in the art, given this Specification containing descriptions of the cytoplasmic, transmembrane and extracellular domains of HARE and various variants of HARE (as discussed in more detail herein below in the Example), should be able to identify and select portions of the HARE protein (e.g., the extracellular domain of HARE or portions thereof, such as an HA-binding domain of HARE) which retain the ability to bind at least one of HA, chondroitin and chondroitin sulfate.

The term “chondroitin sulfate” as used herein will be understood to include any glycosaminoglycan derived from the polymer D-glucuronic acid-β-(1-3)D-N-acetyl galactosamine-β-(1-4), that is sulphated at at least one position selected from positions 4 and 6 of N-acetyl galactosamine and position 2 of glucuronic acid. Table VI lists various GAGs designated as “CS” that fall within the term “chondroitin sulfate” as used in accordance with the present invention, such as but not limited to, CS-A, CS-C, CS-D, and CS-E. The term “chondroitin sulfate” as used herein will also be understood to include GAGs derived from the polymer above in which the glucuronic acid has been epimerized to iduronic acid, such as but not limited to, CS-B or dermatan sulfate.

As used herein, the terms “nucleic acid segment”, “DNA sequence”, “DNA segment” and “nucleic acid sequences” are used interchangeably and refer to a DNA molecule which has been isolated free of total genomic DNA of a particular species. Therefore, a “purified” DNA or nucleic acid segment as used herein refers to a DNA segment which contains a HA Receptor for Endocytosis (“HARE”) coding sequence or fragment or variant thereof yet is isolated away from, or purified free from, unrelated genomic DNA, for example, mammalian host genomic DNA. Included within the term “DNA segment”, are DNA segments and smaller fragments of such segments, and also recombinant vectors, including, for example, plasmids, cosmids, phage, viruses, and the like.

The term “vector” as used herein refers to a nucleic acid molecular capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Similarly, a DNA segment comprising an isolated or purified HARE gene refers to a DNA segment including HARE coding sequences isolated substantially away from other naturally occurring genes or protein encoding sequences. In this respect, the term “gene” is used for simplicity to refer to a functional protein, polypeptide or peptide encoding unit. As will be understood by those skilled in the art, this functional term includes genomic sequences, cDNA sequences or combinations thereof. “Isolated substantially away from other coding sequences” means that the gene of interest, in this case HARE or a fragment thereof, forms the significant part of the coding region of the DNA segment, and that the DNA segment does not contain large portions of naturally-occurring coding DNA, such as large chromosomal fragments or other functional genes or DNA coding regions. Of course, this refers to the DNA segment as originally isolated, and does not exclude genes or coding regions later added to or intentionally left in the segment by the hand of man.

Preferably, DNA sequences in accordance with the present invention will further include genetic control regions which allow for the expression of the sequence in a selected recombinant host. Of course, the nature of the control region employed will generally vary depending on the particular use (e.g., cloning host) envisioned. One of ordinary skill in the art, given this Specification, would be able to identify and select genetic control regions which can be utilized in accordance with the present invention to enhance expression of a HARE gene. Examples of specific genetic control regions which may be utilized are described in more detail herein below with regard to specific recombinant host cells.

In particular embodiments, the invention concerns the use of isolated DNA segments and recombinant vectors incorporating DNA sequences which encode a HARE gene or a variant or fragment thereof, that includes within its amino acid sequence an amino acid sequence in accordance with at least a portion of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 or SEQ ID NO:96. Moreover, in other particular embodiments, the invention concerns isolated DNA segments and recombinant vectors incorporating DNA sequences which encode a gene that includes within its DNA sequence the DNA sequence of a HARE gene or variant or fragment thereof, and in particular to a HARE gene or cDNA or fragment or variant thereof, corresponding to human HARE. For example, where the DNA segment or vector encodes a full length HARE protein, or is intended for use in expressing the HARE protein, preferred sequences are those which are essentially as set forth in SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96. In an alternative embodiment, where the DNA segment may encode a functional portion or variant of the HARE protein, such as a soluble form of the protein or a splice variant of the protein which still retains the ability to bind at least one of HA, chondroitin and chondroitin sulfate, for example a peptide containing an extracellular domain of HARE or an HA-binding domain of HARE, preferred sequences are at least a portion of those which are essentially as set forth in SEQ ID NO:2, SEQ ID NO:4 or SEQ ID NO:96, such as but not limited to SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92 or SEQ ID NO:94. It is within the abilities of one of ordinary skill in the art, given this Specification, to identify the DNA segments encoding the cytoplasmic, transmembrane and extracellular domains of the HARE protein and to locate and select the portions of the amino acid sequences of SEQ ID NO:2, SEQ ID NO:4 or SEQ ID NO:96 which encode the extracellular domain of HARE, or a portion thereof, and not the cytoplasmic or transmembrane domain of HARE. It is also within the abilities of one of ordinary skill in the art, given this Specification, to identify, locate and select domains or regions of the HARE protein that encode at least one of an HA-binding site, a chondroitin-binding site, and a chondroitin sulfate-binding site, as well as portions thereof.

Nucleic acid segments having functional HARE activity may be isolated by the methods described herein. For the purposes of example, the term “a sequence essentially as set forth in SEQ ID NO:2” means that the sequence substantially corresponds to at least a portion of SEQ ID NO:2 and has relatively few amino acids which are not identical to, or a biologically functional equivalent of, the amino acids of SEQ ID NO:2. The term “biologically functional equivalent” is well understood in the art and is further defined in detail herein as a gene having a sequence essentially as set forth in SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95, and that is associated with the ability to bind and endocytose at least one of HA, chondroitin and chondroitin sulfate.

One of ordinary skill in the art would appreciate that a nucleic acid segment encoding a functionally active HARE may contain conserved or semi-conserved amino acid substitutions to the sequences set forth in SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96 and yet still be within the scope of the invention.

In particular, the art is replete with examples of practitioner's ability to make structural changes to a nucleic acid segment (i.e. encoding conserved or semi-conserved amino acid substitutions) and still preserve its enzymatic or functional activity. See for example: (1) Risler et al., (1988) [“ . . . according to the observed exchangeability of amino acid side chains, only four groups could be delineated; (i) Ile and Val; (ii) Leu and Met, (iii) Lys, Arg, and Gln, and (iv) Tyr and Phe.”]; (2) Niefind et al., (1991) [similarity parameters allow amino acid substitutions to be designed]; and (3) Overington et al., (1992) [“Analysis of the pattern of observed substitutions as a function of local environment shows that there are distinct patterns . . . ” Compatible changes can be made.], the contents of all of which are hereby expressly incorporated herein by reference. Standardized and accepted functionally equivalent amino acid substitutions are presented in Table I.

These references and countless others indicate that one of ordinary skill in the art, given a nucleic acid sequence, could make substitutions and changes to the nucleic acid sequence without changing its functionality. Also, a substituted nucleic acid segment may be highly similar and retain its functional activity with regard to its unadulterated parent, and yet still fail to hybridize thereto under standard stringent hybridization conditions. However, while hybridization may not occur at such stringent hybridization conditions, hybridization may be observed at less stringent, relaxed hybridization conditions. Stringent and relaxed hybridization conditions are discussed in more detail herein below.

In addition to naturally-occurring allelic and splice variants of the HARE sequences that may exist in the population, the skilled artisan will further appreciate that changes can be introduced by mutation into the nucleotide sequences described herein, thereby leading to changes in the amino acid sequences of the encoded HARE proteins, without altering the functional ability of said HARE proteins. For example, nucleotide substitutions leading to amino acid substitutions at “non-essential” amino acid residues can be made in the sequence of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95. A “non-essential” amino acid residue is a residue that can be altered from the wild-type sequences of the HARE proteins without altering their biological activity, whereas an “essential” amino acid residue is required for such biological activity. Amino acids for which conservative substitutions can be made are well-known within the art, as described herein.

TABLE I

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, | Glycine, Serine, Threonine, |
| R Groups | Cysteine, Asparagine, Glutamine |
| Negatively Charged | Aspartic Acid, Glutamic Acid |
| R Groups | |
| Positively Charged | Lysine, Arginine, Histidine |
| R Groups | |

Another preferred embodiment of the present invention pertains to nucleic acid molecules encoding HARE proteins that contain changes in amino acid residues that are not essential for activity. Such HARE proteins differ in amino acid sequence from SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 or SEQ ID NO:96 yet retain biological activity. In one embodiment, the isolated nucleic acid molecule comprises a nucleotide sequence encoding a protein, wherein the protein comprises an amino acid sequence at least about 45% homologous to the amino acid sequences of at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 or SEQ ID NO:96. Preferably, the protein encoded by the nucleic acid molecule is at least about 60% homologous to at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96; more preferably at least about 70% homologous to at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96; still more preferably at least about 80% homologous to at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96; even more preferably at least about 90% homologous to at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96; and most preferably at least about 95% homologous to at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96.

An isolated nucleic acid molecule encoding a HARE protein homologous to the protein of at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96 can be created by introducing one or more nucleotide substitutions, additions or deletions into the nucleotide sequence of at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95, such that one or more amino acid substitutions, additions, or deletions are introduced into the encoded protein. Mutations can be introduced into the sequences by standard techniques well known in the art, such as but not limited to, site-directed mutagenesis and PCR-mediated mutagenesis. Alternatively, mutations can be introduced randomly along all or part of a HARE coding region, such as but not limited to, by transposon mutagenesis or saturation mutagenesis, and the resultant mutants screened for HARE biological activity to identify mutants that retain activity. Following mutagenesis, the encoded protein containing one or more mutations can be expressed by any recombinant technology known in the art or described herein, and the activity of the HARE protein can then be determined.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package (see Needleman and Wunsch (1970)). Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (i.e., encoding) part of the DNA sequence shown in any one or more of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least about 85% identity and often about 90% to about 95% sequence identity, more usually at least about 99% sequence identity as compared to a reference sequence over a comparison region. Similar calculations are used when comparing amino acid residues in polypeptide sequences.

Another preferred embodiment of the present invention is the use of a purified nucleic acid segment that encodes a protein in accordance with at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96, further defined as a recombinant vector. As used herein, the term “recombinant vector” refers to a vector that has been modified to contain a nucleic acid segment that encodes a HARE protein, or variant or fragment thereof, such as a soluble form of the protein or an HA-binding domain of the protein or a splice variant of the protein. The recombinant vector may be further defined as an expression vector comprising a promoter operatively linked to said nucleic acid segment encoding HARE, a variant thereof or a fragment thereof. That is, the nucleic acid segment is in a form suitable for expression of the nucleic acid in a host cell, which means that the recombinant expression vectors include one or more regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., HARE proteins, mutant forms of HARE proteins, fusion proteins, etc.).

Yet another preferred embodiment of the present invention is the use of a purified nucleic acid segment that encodes an active portion of the protein in accordance with a portion of at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96. For example, the invention also includes utilization of a purified nucleic acid segment encoding a variant of the protein such as a soluble form of the protein (i.e., a portion of the protein containing the extracellular domain but not the cytoplasmic or transmembrane domains of the protein) which retains the ability to bind at least one of HA, chondroitin and chondroitin sulfate, or a portion of the protein containing an active HA-binding domain of HARE.

A further preferred embodiment of the present invention utilizes a host cell, made recombinant with a recombinant vector comprising a gene encoding HARE or a variant or fragment thereof. In a preferred embodiment, the recombinant host cell is a eukaryotic cell. As used herein, the term “engineered” or “recombinant” cell is intended to refer to a cell into which a recombinant gene, such as a gene encoding HARE or a variant or fragment thereof, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter not naturally associated with the particular introduced gene. In a preferred embodiment, the recombinantly introduced gene may be integrated into the genome of the host cell.

Where one desires to use a eucaryotic host system, such as yeast or Chinese hamster ovary, African green monkey kidney cells, VERO cells, or the like, it will generally be desirable to bring the gene encoding HARE or a variant or fragment thereof under the control of sequences which are functional in the selected alternative host. In another alternative, the vector may contain a cassette which signals for the sequence to be integrated into the chromosome. The appropriate DNA control sequences, as well as their construction and use, are generally well known in the art as discussed in more detail herein below.

In preferred embodiments, the DNA segments encoding HARE or a variant or fragment thereof further include DNA sequences, known in the art functionally as origins of replication or “replicons”, which allow replication of contiguous sequences by the particular host. Such origins allow the preparation of extrachromosomally localized and replicating chimeric segments or plasmids, to which HARE DNA sequences are ligated. In one instance, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The isolation and use of other replication origins such as the SV40, polyoma or bovine papilloma virus origins, which may be employed for cloning or expression in a number of higher organisms, are well known to those of ordinary skill in the art. In certain embodiments, the invention may thus be defined in terms of a recombinant transformation vector which includes the HARE coding gene sequence (or HARE variant or fragment coding gene sequence) together with an appropriate replication origin and under the control of selected control regions.

Thus, it will be appreciated by those of skill in the art that other methods may be used to obtain the gene or cDNA encoding HARE or a variant or fragment thereof, in light of the present disclosure. For example, polymerase chain reaction or RT-PCR produced DNA fragments may be obtained which contain full complements of genes or cDNAs from a number of sources, including other eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HARE or portion or variant thereof.

Once the DNA has been isolated, it is ligated together with a selected vector. Virtually any cloning vector can be employed to realize advantages in accordance with the invention. Typical useful vectors include plasmids, cosmids, phages and viral vectors for use in prokaryotic or eukaryotic organisms. Examples include pKK223-3, pSA3, pcDNA3.1, recombinant lambda, SV40, polyoma, adenovirus, bovine papilloma virus and retroviruses.

One procedure that would further augment HARE gene copy number is the insertion of multiple copies of the gene into the vector. Another technique would include integrating the HARE gene or multiple copies thereof into chromosomal DNA.

Where a eukaryotic source such as tissues rich in sinusoidal cells of the reticuloendothelial system such as liver, spleen, lymph node and bone marrow is employed, one will desire to proceed initially by preparing a cDNA library or obtaining a cDNA pool. This is carried out first by isolation of mRNA from the above cells, followed by preparation of double stranded cDNA using an enzyme with reverse transcriptase activity and ligation with the selected vector. Numerous possibilities are available and known in the art for the preparation of the double stranded cDNA, and all such techniques are believed to be applicable. A preferred technique involves reverse transcription. If a cDNA library is used, once a population of double stranded cDNAs is obtained, a cDNA library is prepared in the selected host by accepted techniques, such as by ligation into the appropriate vector and amplification in the appropriate host. Due to the high number of clones that are obtained, and the relative ease of screening large numbers of clones by the techniques set forth herein, one may desire to employ phage expression vectors, such as λgt11, λgt12, λGem11, pCR-XL-TOPO, pSecTag/FRT/V5-His-TOPO, and/or AZAP for the cloning and expression screening of cDNA clones.

In certain other embodiments, the invention concerns isolated DNA segments and recombinant vectors that include within their sequence a nucleic acid sequence essentially as set forth in at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95. For purposes of example, the term “essentially as set forth in SEQ ID NO:1” is used in the same sense as described above and means that the nucleic acid sequence substantially corresponds to a portion of SEQ ID NO:1 and has relatively few codons which are not identical, or functionally equivalent, to the codons of SEQ ID NO:1. The term “functionally equivalent codon” is used herein to refer to codons that encode the same amino acid, such as the six codons for arginine or serine, and also refers to codons that encode biologically equivalent amino acids. The term “essentially as set forth in SEQ ID NO:1” also incorporates the concept that the encoded protein is functionally equivalent to the protein encoded by SEQ ID NO:1. Thus, pursuant to In Re Wands, Applicants herein disclose conditions and criteria to describe alternate embodiments that could be easily and repeatably determined by one of ordinary skill in the art.

It will also be understood that amino acid and nucleic acid sequences may include additional residues, such as additional N- or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and receptor activity (i.e., HA, chondroitin or chondroitin sulfate binding) is concerned. The addition of terminal sequences particularly applies to nucleic acid sequences which may, for example, include various non-coding sequences flanking either of the 5′ or 3′ portions of the coding region or may include various internal sequences, which are known to occur within genes. The HARE proteins and variants and fragments thereof described herein are derived from larger precursor proteins, and therefore such precursor proteins also fall within the scope of the present invention.

Allowing for the degeneracy of the genetic code as well as conserved and semi-conserved substitutions, sequences which have between about 40% and about 80%; or more preferably, between about 80% and about 90%; or even more preferably, between about 90% and about 99%; of nucleotides which are identical to the nucleotides of at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95 will be sequences which are “essentially as set forth in SEQ ID NO:1”, “essentially as set forth in SEQ ID NO:3”, “essentially as set forth in SEQ ID NO:55”, “essentially as set forth in SEQ ID NO:57”, “essentially as set forth in SEQ ID NO:59”, “essentially as set forth in SEQ ID NO:61”, “essentially as set forth in SEQ ID NO:63”, “essentially as set forth in SEQ ID NO:65”, “essentially as set forth in SEQ ID NO:67”, “essentially as set forth in SEQ ID NO:69”, “essentially as set forth in SEQ ID NO:71”, “essentially as set forth in SEQ ID NO:73”, “essentially as set forth in SEQ ID NO:75”, “essentially as set forth in SEQ ID NO:77”, “essentially as set forth in SEQ ID NO:79”, “essentially as set forth in SEQ ID NO:81”, “essentially as set forth in SEQ ID NO:83”, “essentially as set forth in SEQ ID NO:85”, “essentially as set forth in SEQ ID NO:87”, “essentially as set forth in SEQ ID NO:89”, “essentially as set forth in SEQ ID NO:91”, “essentially as set forth in SEQ ID NO:93” and “essentially as set forth in SEQ ID NO:95”, respectively. Sequences which are essentially the same as those set forth in SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95, respectively, may also be functionally defined as sequences which are capable of hybridizing to a nucleic acid segment containing the complement of SEQ ID NO:1 under stringent or relaxed hybridizing conditions. Suitable standard hybridization conditions will be well known to those of skill in the art and are clearly set forth herein.

The term “standard hybridization conditions” as used herein is used to describe those conditions under which substantially complementary nucleic acid segments will form standard Watson-Crick base-pairing. A number of factors are known that determine the specificity of binding or hybridization, such as pH, temperature, salt concentration, the presence of agents, such as formamide and dimethyl sulfoxide, the length of the segments that are hybridizing, and the like. When it is contemplated that shorter nucleic acid segments will be used for hybridization, for example fragments between about 14 and about 100 nucleotides, salt and temperature preferred conditions for hybridization will include 1.2-1.8×HPB (High Phosphate Buffer) at 40-50° C. When it is contemplated that longer nucleic acid segments will be used for hybridization, for example fragments greater than 100 nucleotides, salt and temperature preferred conditions for hybridization will include 1.2-1.8×HPB at 60-70° C.

The term “standard hybridization conditions” includes stringent hybridization conditions as well as relaxed hybridization conditions. In general, when the temperature is increased and salt concentration (ionic strength) is decreased in the wash, the conditions become more stringent; these conditions favor hybrid interactions that have a higher degree of complementarity. When the annealing and wash conditions are at lower temperature and higher ionic strength, less complementary hybrids, which might not be present under more stringent conditions, can be stabilized. For example, to screen the λ-ZAP EXPRESS™ rat LECs cDNA library relatively high-stringency conditions (60° C. overnight in QUIKHYB® hybridization solution (Stratagene, La Jolla, Calif.) followed by two washes for 15 minutes each at room temperature with 2×SSC, 0.1% SDS and two washes for 30 minutes each at 50° C. with 0.1×SSC, 0.1% SDS) were used. However, less stringent hybridization conditions were used to screen a genomic DNA library that was expected to contain numerous exons separated by noncomplementary introns (40° C. overnight in QUIKHYB™ hybridization solution, two washes for 15 minutes each at room temperature with 2×SSC, 0.1% SDS and one wash for 30 minutes at 40° C. with 0.1×SSC-0.1% SDS).

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a nucleotide sequence of the present invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Naturally, the present invention also encompasses DNA segments which are complementary, or essentially complementary, to the sequence set forth in at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment of at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95.

The term “binding” as used herein refers to the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

The present invention also includes primers which may be utilized to amplify the coding region of HARE or portions thereof. Nucleic acid segments capable of hybridizing to at least one of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95 in accordance with the present invention are described in copending application U.S. Ser. No. 09/842,930, which has previously been incorporated by reference herein. However, it is to be understood that the present invention is not limited to such primers, and a person of ordinary skill in the art, given this Specification, will be able to identify and select primers which can be utilized to amplify the coding region of HARE, or a portion thereof, such as an extracellular domain or an HA-binding domain of HARE. The present invention also includes primers which are engineered to introduce a restriction site into a DNA sequence to aid in cloning of such DNA sequence. Examples are provided in copending application U.S. Ser. No. 09/842,930 (previously incorporated by reference). However, it is within the skill of one in the art to create restriction sites in a DNA segment which aid in ligation of such DNA segment to a vector having a particular cloning site consisting of a set of restriction sites, and therefore, the present invention is not limited to the primers listed herein.

The nucleic acid segments of the present invention, regardless of the length of the coding sequence itself, may be combined with other DNA sequences, such as promoters, polyadenylation signals, additional restriction enzyme sites, multiple cloning sites, epitope tags, poly histidine regions, membrane insertion signal sequences, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

Naturally, it will also be understood that this invention is not limited to the particular nucleic acid sequences of SEQ ID NO:1, SEQ ID NO:3, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93 and SEQ ID NO:95 and amino acid sequences of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96. Recombinant vectors and isolated DNA segments may therefore variously include the HARE coding regions themselves, coding regions that encode binding domains for at least one of HA, chondroitin and chondroitin sulfate, coding regions bearing selected alterations or modifications in the basic coding region, or they may encode larger polypeptides which nevertheless include HARE-coding regions or may encode biologically functional equivalent or precursor proteins or peptides which have variant amino acids sequences.

The DNA segments of the present invention encompass biologically functional equivalent HARE proteins, portions thereof that bind at least one of HA, chondroitin and chondroitin sulfate, and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the functional activity or to antigenicity of the HARE protein.

A preferred embodiment of the present invention utilizes a purified composition comprising a polypeptide having an amino acid sequence in accordance with at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96. The term “purified” as used herein, is intended to refer to a HARE protein composition, wherein the HARE protein, fragment thereof or variant thereof, or appropriately modified HARE protein, fragment or variant thereof (e.g. containing a [HIS]₆ tail) is purified to any degree relative to its naturally-obtainable state. Preferably, the term “isolated” or “purified” polypeptide or protein or biologically-active protein thereof is substantially free of cellular material or other contaminating proteins from the cell or tissue source from which the HARE protein or variant or fragment thereof is derived. The phrase “substantially free of cellular material” as used herein includes preparations of HARE proteins, fragments thereof and variants thereof in which the protein is separated from cellular components of the cells from which it is isolated or recombinantly-produced. In one embodiment, the phrase “substantially free of cellular material” includes preparations of HARE proteins, fragments or variants thereof having less than about 30% (by dry weight) on non-HARE proteins (also referred to herein as a “contaminating protein”), more preferably less than about 20% of non-HARE proteins, still more preferably less than about 10% of non-HARE proteins, and most preferably less than about 5% of non-HARE proteins. When the HARE protein, fragment or variant thereof is recombinantly-produced, it is also preferably substantially free of culture medium, i.e., culture medium represent less than about 20%, more preferably less than about 10%, and most preferably less than about 5% of the volume of the HARE protein preparation.

The invention also utilizes a purified composition comprising a polypeptide having an amino acid sequence in accordance with a portion of at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96 wherein the polypeptide is capable of selectively binding at least one of HA, chondroitin and chondroitin sulfate. The ligand blot assay described in detail and utilized in copending application U.S. Ser. No. 09/842,930 (previously incorporated by reference) may be utilized to assay for such an HA-binding domain, chondroitin-binding domain and/or chondroitin sulfate-binding domain of HARE. However, such assay is an indirect assay of HA/chondroitin/chondroitin sulfate binding to HARE. Optionally, a direct binding assay is described in detail herein, which utilizes a biotinylated GAG binding assay based on an ELISA-like format. Such direct binding assay may be utilized in accordance with the present invention to assay for such an HA-binding domain, chondroitin-binding domain and/or chondroitin sulfate-binding domain of HARE.

Turning to the expression of the HARE gene whether from genomic DNA, or a cDNA, one may proceed to prepare an expression system for the recombinant preparation of the HARE protein or a variant thereof. The engineering of DNA segment(s) for expression in a eukaryotic system may be performed by techniques generally known to those of skill in recombinant expression or as described in detail herein below in the methods section for creation of stable cell lines as well as expression of DNA segments in such stable cell lines.

Another embodiment of the present invention utilizes a method of preparing a protein composition comprising growing a recombinant host cell comprising a vector that encodes a protein which includes an amino acid sequence in accordance with at least one of SEQ ID NO:2, SEQ ID NO:4, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94 and SEQ ID NO:96 or an amino acid sequence which is functionally similar with conserved or semi-conserved amino acid changes. The host cell will be grown under conditions permitting nucleic acid expression and protein production followed by recovery of the protein so produced. The production of HARE, including the host cell, conditions permitting nucleic acid expression, protein production and recovery will be known to those of skill in the art in light of the present disclosure of the HARE gene, and the HARE gene protein product HARE, and by the methods described herein.

It is similarly believed that almost any eukaryotic expression system may be utilized for the expression of HARE e.g., baculovirus-based, glutamine synthase-based, dihydrofolate reductase-based systems, SV-40 based, adenovirus-based, cytomegalovirus-based, yeast-based, and the like, could be employed. For expression in this manner, one would position the coding sequences adjacent to and under the control of a promoter. It is understood in the art that to bring a coding sequence under the control of such a promoter, one positions the 5′ end of the transcription initiation site of the transcriptional reading frame of the protein between about 1 and about 50 nucleotides “downstream” of (i.e., 3′ of) the chosen promoter.

Where eukaryotic expression is contemplated, one will also typically desire to incorporate into the transcriptional unit which includes the HARE gene or DNA, an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

It is contemplated that virtually any of the commonly employed host cells can be used in connection with the expression of HARE in accordance herewith. Examples of preferred cell lines for expressing HARE cDNA of the present invention include cell lines typically employed for eukaryotic expression such as 239, AtT-20, HepG2, VERO, HeLa, CHO, WI 38, BHK, COS-7, 293, RIN and MDCK cell lines. This will generally include the steps of providing a recombinant host bearing the recombinant DNA segment encoding a functionally active HARE or an active peptide fragment or variant thereof and capable of expressing the functionally active HARE or the active peptide fragment or variant thereof; culturing the recombinant host under conditions that will allow for expression of the recombinant DNA segment; and separating and purifying the functionally active HARE protein or the active peptide fragment or variant thereof which is able to specifically bind at least one of HA, chondroitin and chondroitin sulfate from the recombinant host.

Generally, the conditions appropriate for expression of the cloned HARE gene or cDNA will depend upon the promoter, the vector, and the host system that is employed. For example, tetracycline induction may be employed. Where other promoters are employed, different materials may be needed to induce or otherwise up-regulate transcription.

The present invention further utilizes antibodies raised against the Hyaluronan Receptor for Endocytosis (HARE) proteins or fragments thereof described herein, and which are able to selectively bind an epitope of the HARE. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies includes, but are not limited to, polyclonal, monoclonal, chimeric, single chain, F_{ab}, F_{ab′}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as but not limited to, IgG₁, IgG₂, and the like. Reference herein to antibodies includes a reference to all such classes, subclasses and types of human antibody species.

An isolated HARE protein of the invention, or a portion or fragment thereof, may be intended to serve as an antigen, and additionally can be used as an immunogen to generate antibodies that immunospecifically bind the antigen, using standard techniques for polyclonal and monoclonal antibody preparation. The full-length protein can be used or, alternatively, an antigenic derivative, fragment, variant, analog, homolog or ortholog thereof may be utilized as an immunogen in the generation of antibodies that immunospecifically bind these protein components. An antigenic peptide fragment comprises at least six amino acid residues of the amino acid sequence of the full length protein and encompasses an epitope thereof such that an antibody raised against the peptide forms a specific immune complex with the full length protein or with any fragment or variant that contains the epitope. Preferably, the antigenic peptide comprises at least 10 amino acid residues, at least 15 amino acid residues, at least 20 amino acid residues or at least 30 amino acid residues.

In one instance, binding of the antibody to the HARE inhibits the binding of at least one of HA, chondroitin and chondroitin sulfate to HARE and subsequently prevents endocytosis by cells of at least one of HA, chondroitin and chondroitin sulfate by the HARE. Methods of producing such antibodies generally involve immunizing a non-human animal with an immunogenic fragment of the HARE protein. In a preferred embodiment, the immunogenic fragment may comprise an HA-binding domain of HARE. Methods of producing such antibodies are well known to a person of ordinary skill in the art, and therefore no further description is required.

In another instance, the antibody described herein above may bind to at least one of a purified HARE protein or a variant thereof and inhibit the binding of at least one of HA, chondroitin and chondroitin sulfate to the purified HARE protein and/or variant. It is possible that the binding of the antibody to the purified HARE protein or variant may inhibit binding of one of HA, chondroitin and chondroitin sulfate, while not affecting the ability of the one or two of the other GAGs to bind the purified HARE protein or variant. Given the disclosure of the present invention, one of ordinary skill in the art could construct a detection assay using such an antibody and purified HARE protein or variant to measure multiple GAG content in a sample as well as the content of the GAG(s) that binds in the presence of the blocking antibody.

In a preferred embodiment, the antibody utilized in the methods of the present invention is a monoclonal antibody. The terms “monoclonal antibody”, “mAb” and “monoclonal antibody composition” as used herein refer to a homogenous preparation of antibody molecules, produced by a hybridoma cell line, all of which exhibit the same primary structure and antigenic specificity. That is, the monoclonal antibodies are a population of antibody molecules that contain only one molecular species of antibody molecule consisting of a unique light chain gene product and a unique heavy chain gene product. In particular, the complementarity determining regions (CDRs) of the monoclonal antibodies are identical in all the molecules of the population, and the mAbs thus contain an antigen binding site capable of immunoreacting with a particular epitope of the antigen characterized by a unique binding affinity for it. In the present invention, all of the antibody molecules of a particular monoclonal antibody preparation recognize and selectively bind the same epitope of HARE.

The monoclonal antibodies are produced by methods generally well known to a person of ordinary skill in the art, such as those described in Kohler and Milstein, (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro, and briefly involve culturing the hybridoma cell producing the monoclonal antibody specific for HARE under conditions that permit production of such monoclonal antibody.

The immunizing agent will typically include the protein antigen, a fragment thereof, a variant thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, (1986)). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, (1984); Brodeur et al., (1987)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The monoclonal antibodies of the presently disclosed and claimed invention may be utilized to purify functionally active HARE from a biological sample containing HARE via affinity purification. In preferred embodiments, the biological sample may be a tissue rich in sinusoidal cells of the reticuloendothelial system, such as at least one of liver, spleen, lymph nodes and bone marrow. However, it is to be understood that the biological sample may be any sample containing a functionally active HARE.

Affinity purification of proteins utilizing antibodies raised against such proteins is well known to a person of ordinary skill in the art. Briefly, an affinity matrix comprising a monoclonal antibody of the present invention bound to a solid support may be produced by methods well known in the art, and the biological sample may be contacted with the affinity matrix such that HARE in the biological sample binds to the monoclonal antibody of the affinity matrix. The HARE bound to the monoclonal antibody of the affinity matrix may be separated from the remainder of the biological sample by methods well known in the art. The HARE protein is then released from the monoclonal antibody of the affinity matrix and eluted from the affinity column by the addition of a solution, referred to as an eluate, which disrupts the binding between the HARE protein and the antibody. Such eluates are well known in the art, and may include solutions having a lower pH, solutions having a higher salt concentration, and the like. In preferred embodiments, the solution utilized for elution of the HARE protein is based on the ability of the solution to retain the functional activity of the HARE protein. That is, exposure to low pH or high salt may affect the conformations of some proteins, and therefore an eluate is chosen that does not have any effect on the activity of the protein to be eluted.

The monoclonal antibodies of the present invention can also be used to affinity purify peptide fragments or variants of HARE proteins as long as the peptide fragment or variant contains the epitope against which the monoclonal antibody was raised. The monoclonal antibodies of the present invention may also be utilized to affinity purify other proteins (such as the “HARE-like” proteins described herein above) that contain at least one domain or motif similar to a domain or motif of a HARE protein, as long as the corresponding HARE protein domain or motif contains the epitope against which the monoclonal antibody was raised.

In another embodiment of the present invention, a method of identifying compounds which inhibit binding of at least one of HA, chondroitin and chondroitin sulfate to HARE is provided. The method includes providing a purified fragment of HARE capable of binding at least one of HA, chondroitin and chondroitin sulfate, such as a soluble form or variant of HARE, and forming a first affinity matrix comprising the purified fragment of HARE bound to a solid support. The first affinity matrix is separated into two portions, and a test compound is contacted with one portion of the first affinity matrix, thereby forming a treated affinity matrix. In two parallel experiments, at least one of HA, chondroitin and chondroitin sulfate that is labeled in such a manner that it can be readily detected is contacted with: (1) the second portion of the first affinity matrix, and (2) the treated affinity matrix. If the HA, chondroitin or chondroitin sulfate binds to a greater extent to the first affinity matrix than to the treated affinity matrix, a determination that the test compound inhibits binding of HA, chondroitin or chondroitin sulfate to HARE can be made. The purified fragment of HARE may be a soluble fragment of HARE, such as an extracellular domain of HARE or an HA-binding domain of HARE, or a variant of HARE. It is within the abilities of a person having ordinary skill in the art to design a high throughput ELISA-like assay to detect such derived mimetics by using adsorbed purified HARE variant protein and a biotin-GAG binding assay as described in detail herein.

In yet another embodiment of the present invention, a method of treating a liquid solution containing at least one of HA, chondroitin and chondroitin sulfate is provided. Such method includes providing an affinity matrix comprising a functionally active fragment or variant of HARE, as described herein above, bound to a solid support, and exposing a quantity of the liquid solution to the affinity matrix wherein at least one of HA, chondroitin and chondroitin sulfate contained in the liquid solution is removed therefrom. Such liquid solution could be blood or plasma, such as when blood or plasma is removed from a dialysis patient and filtered to remove contaminants and waste.

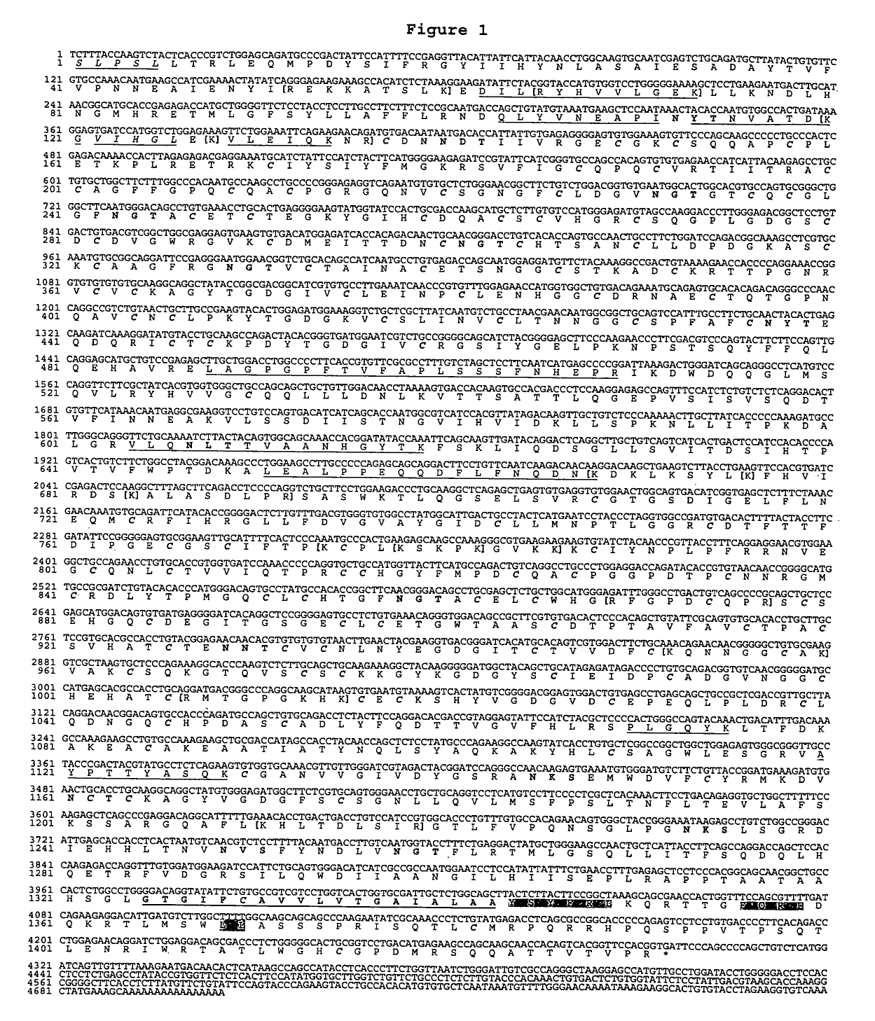

The present invention utilizes the characterization and molecular description of the rat and human HAREs (as described herein below in reference to FIGS. 1-15 and 18-25 and in parent applications U.S. Ser. Nos. 09/842,930 and 10/133,172) to develop novel strategies to interfere with the metastatic process. In addition, many therapeutic and diagnostic utilities for a functionally active HARE or active peptide fragment or variant thereof, a plasmid or chromosomally integrated gene encoding same and antibodies which bind thereto are envisioned by the present invention. Such utilities are described in detail hereinbelow. However, various therapies and diagnostic assays utilizing the nucleic acid and amino acid sequences, functionally active peptides and proteins, and antibodies of the present invention can be envisioned, and therefore the present invention is not limited to the methods described hereinbelow.

The monoclonal antibodies (raised against the rat HARE) of the present invention can be utilized in a mammal, such as a human, to target a compound deleterious to tumor cells, such as a radioisotope or chemotherapeutic agent, to such tumor cells when the cancer is present in tissues that express HARE, such as lymph nodes, bone marrow, liver and spleen. When the mammal is a human, the mAb is humanized as described herein and conjugated to the compound/radioisotope/chemotherapeutic agent, and an effective amount of such conjugate is then administered to the individual such that the mAb selectively binds to cells expressing HARE on a surface thereof, thereby delivering the compound/radioisotope/chemotherapeutic agent to the nearby tumor cells which are in close proximity to the cells expressing HARE on the surface thereof.

The mAb/compound conjugate or blocking Ab can be targeted to tissues such as lymph node, bone marrow and liver to minimize the chance of metastasis during surgery to remove a primary tumor. The mAb/compound conjugate or blocking Ab can also be administered and directed to HARE in such tissues after there is evidence for metastasis.

A similar method can be utilized when it is desired to target a non-deleterious compound to cells expressing HARE on a surface thereof. As in the previous example, the compound is conjugated to a monoclonal antibody of the present invention, and the compound-monoclonal antibody conjugate is administered in an effective amount to a mammal such that the monoclonal antibody selectively binds to cells expressing HARE on a surface thereof, thereby delivering the compound to such cells.

Such utilization of the monoclonal antibodies of the present invention may require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the present invention can be “humanized”, that is, the antibodies are engineered such that antigenic portions thereof are removed and like portions of a human antibody are substituted therefor, while the antibodies' affinity for an epitope of HARE is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, the Specifications of which are all hereby expressly incorporated herein by reference in their entirety.

Humanized forms of antibodies are chimeric immunoglobulins, immunoglobulin chains or fragments thereof (such as Fᵥ, F_{ab}, F_{ab′}, F_{(ab′)2} or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., (1986); Riechmann et al., (1988); Verhoeyen et al., (1988)), by substituting rodent CDRs or CDR sequences for the corresponding sequences of a human antibody. (See also U.S. Pat. No. 5,225,539.) In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, (1992)).

97 published articles relating to the generation or use of humanized antibodies were identified by a PubMed search of the database as of Apr. 25, 2002. Many of these studies teach useful examples of protocols that can be utilized with the present invention, such as Sandborn et al., (2001); Mihara et al., (2001); Yenari et al., (2001); Morales et al., (2000); Richards et al., (1999); Yenari et al., (1998); and Shinkura et al., (1998), all of which are expressly incorporated in their entirety by reference. For example, a treatment protocol that can be utilized in such a method includes a single dose, generally administered intravenously, of 10-20 mg of humanized mAb per kg (Sandborn, et al. (2001)). In some cases, alternative dosing patterns may be appropriate, such as the use of three infusions, administered once every two weeks, of 800 to 1600 mg or even higher amounts of humanized mAb (Richards et al., (1999)). However, it is to be understood that the invention is not limited to the treatment protocols described above, and other treatment protocols which are known to a person of ordinary skill in the art may be utilized in the methods of the present invention.

The presently disclosed and claimed invention further includes fully human monoclonal antibodies against the HARE protein or portions thereof. Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., (1983)) and the EBV hybridoma technique to produce human monoclonal antibodies (see Cole, et al., (1985)). Human monoclonal antibodies may be utilized in the practice of the present invention and may be produced by using human hybridomas (see Cote, et al., (1983)) or by transforming human B-cells with Epstein Barr Virus in vitro (see Cole, et al., (1985)).

In addition, human antibodies can also be produced using additional techniques, including phage display libraries (Hoogenboom and Winter, (1991); Marks et al., (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., (1992); Lonberg et al., (1994); Morrison, (1994); Fishwild et al., (1996); Neuberger, (1996); and Lonberg and Huszar, (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the XENOMOUSE™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

The monoclonal antibodies of the presently disclosed and claimed invention may also be utilized in a method of preventing metastasis in an individual wherein the tumor cells of such individual are provided with an HA, chondroitin sulfate or chondroitin coat which interacts with non-tumor cells expressing HARE on a surface thereof. The monoclonal antibody may be humanized as described herein, and an effective amount of the humanized monoclonal antibody can then be administered to the individual such that the humanized monoclonal antibody selectively binds to an epitope of HARE expressed on the surface of the non-tumor cells and inhibits binding of at least one of HA, chondroitin sulfate and chondroitin in the coat of the tumor cells to the non-tumor cells expressing HARE.

An exemplary treatment protocol for use in such a method includes a single dose, generally administered intravenously, of about 10 mg of humanized mAb per kg to about 20 mg of humanized mAb per kg (Sandborn et al. (2001)). In some cases, alternative dosing patterns may be appropriate, such as the use of three infusions, administered once every two weeks, of about 800 μg to about 1600 μg or even higher amounts of humanized mAb (Richards et al., (1999)).

More effective results can be obtained in some patients with a dose in the range of from about 5 mg/kg to about 20 mg/kg taken weekly and administered by subcutaneous injection or by use of an automated delivery device as used for delivery of insulin. However, it is to be understood that the invention is not limited to the treatment protocols described herein above, and other treatment protocols which are known to a person of ordinary skill in the art may be utilized in the methods of the present invention.

While such methods described above involve preventing metastasis by preventing interaction between tumor cells having an HA, chondroitin or chondroitin sulfate coat and non-tumor cells expressing HARE on a surface thereof, the present invention is not limited to such use, and the method described herein above may be utilized to prevent or to modify interactions between any cell having an HA, chondroitin or chondroitin sulfate coat and a cell expressing HARE on a surface thereof. Optionally, administration of a soluble HARE variant may be utilized to intentionally block or modify the interaction between cells. For example, a variant could stimulate stem cell homing to bone marrow and thus increase red or white blood cell production.

A similar method encompassed by the present invention utilizes a compound other than the humanized monoclonal antibody that inhibits binding of at least one of HA, chondroitin sulfate and chondroitin to HARE or a fragment or variant thereof, such that upon administration of an effective amount of the compound to the individual described above, the compound inhibits binding of at least one of HA, chondroitin sulfate and chondroitin in the coat of tumor cells to non-tumor cells expressing HARE (or fragment or variant thereof) on a surface thereof. For example, such compound may be any compound that acts as a mimetic for the HA binding site, including a mimetic peptide, a nucleic acid, an oligonucleotide or a PNT (a synthetic DNA formed of protein which mimics oligonucleotides), and conjugates thereof, wherein such compound binds to HARE (or fragment or variant thereof) expressed on the surface of non-tumor cells and inhibits binding of at least one of HA, chondroitin sulfate and chondroitin in the coat of tumor cells to non-tumor cells expressing HARE (or fragment or variant thereof). However, the invention is not limited to the use of the compounds described herein above as the compound but rather includes any drug or chemical that inhibits HA binding to HARE (or fragment or variant thereof). Such compounds are identified using an affinity matrix column or multiwell format comprising an HA-, chondroitin sulfate-, or chondroitin-binding domain of HARE bound to a solid support. Upon passing candidate compounds over the immobilized HARE, HA is then passed over the immobilized HARE, and a decrease in HA binding (as detected by methods described herein or known to one of ordinary skill in the art, such as by utilization of HA labeled in such a manner that it can be detected readily) will suggest that such a compound is effective in the method described above.

In a preferred embodiment of the method of preventing interaction between a first cell expressing HARE on a surface thereof and a second cell whose surface contains at least one of an HA, chondroitin and chondroitin sulfate, a functionally active, soluble variant or fragment of HARE is utilized. The functionally active, soluble variant or fragment of HARE is capable of binding at least one of HA, chondroitin and chondroitin sulfate on the surface of the second cell, thereby directly competing with HARE on the surface of the first cell for binding to the HA/chondroitin/chondroitin sulfate on the surface of the second cell. When an effective amount of the functionally active, soluble variant or fragment of HARE is administered, the functionally active, soluble variant or fragment of HARE inhibits binding of the HARE expressed on the first cell to at least one of HA, chondroitin and chondroitin sulfate on the surface of the second cell.

A treatment protocol for use in such a method includes the same or similar protocol for treatment with a humanized mAb as described previously herein above. Such a treatment protocol would utilize a specific mimetic drug (whether a peptide or other chemical or compound) or a soluble variant or fragment of HARE, in the range of from about 5 mg to about 300 mg, and be taken daily and administered by at least one of orally, subcutaneous injection or use of an automated delivery device such as a time release skin patch or a small implanted pump, such as used for delivery of insulin.

While such methods described above involve preventing interaction between tumor cells having HA, chondroitin and/or chondroitin sulfate on a surface thereof and non-tumor cells expressing HARE on a surface thereof, the present invention is not limited to such use, and the method described herein above can be utilized to prevent interactions between any cell having HA, chondroitin and/or chondroitin sulfate on a surface thereof and a cell expressing HARE on a surface thereof.

Another method of the present invention involves targeting a compound to a tissue of a human patient wherein cells of the tissue do not express a functionally active HARE on a surface thereof, but wherein the cells of the tissue express one or more other cell surface or extracellular matrix components capable of binding to HA, chondroitin sulfate or chondroitin, such as but not limited to, CD44. The method involves providing a compound of interest, such as a drug, conjugated to at least one of HA, chondroitin sulfate and chondroitin, which thereby functions as a drug delivery device. By conjugating a drug to HA, chondroitin sulfate or chondroitin and co-administering such conjugate for a therapeutic purpose together with the blocking agents disclosed above to prevent the binding and uptake of HA, chondroitin sulfate or chondroitin to HARE, the lifetime of such drug in the bloodstream or targeted tissues can be prolonged. An effective amount of a humanized monoclonal antibody that selectively binds to an epitope of HARE and inhibits binding of at least one of HA, chondroitin and chondroitin sulfate to HARE, as described in detail herein above, is provided and administered to the human patient such that the humanized monoclonal antibody binds HARE and blocks the binding of at least one of HA, chondroitin sulfate and chondroitin to HARE, so that upon administration of an effective amount of the compound-HA, compound-chondroitin sulfate or compound-chondroitin conjugate to the human patient, the compound-HA, compound-chondroitin sulfate or compound-chondroitin conjugate is not able to bind to the cells expressing HARE and is therefore delivered to the cells of a tissue which do not express HARE on a surface thereof. Optionally, a specific mimetic drug could be utilized in the same manner as described herein for the monoclonal antibody.

A treatment protocol for use in such a method includes the same or similar protocol for treatment with a humanized mAb as described herein above. Optionally, in a treatment protocol utilizing a specific mimetic drug, whether a peptide or other chemical or compound, the specific mimetic drug could be administered in the range of from about 5 mg to about 300 mg taken daily and administered orally, by subcutaneous injection or by use of an automated delivery device such as a time release skin patch or a small implanted pump, such as used for delivery of insulin.

In a similar manner, if one desires to target a compound of interest, such as a drug, to a tissue of an individual wherein cells of the tissue express HARE on a surface thereof, the method above may be utilized with the exception that the humanized monoclonal antibody is omitted. That is, the method includes conjugating the compound to an HA, chondroitin sulfate or chondroitin molecule or a desired combination thereof (which acts as a drug delivery device, as described herein before), and administering an effective amount of the HA-, chondroitin sulfate- and/or chondroitin-compound conjugate to the individual such that the HARE expressed on the surface of cells in the tissue bind and endocytose the HA-, chondroitin sulfate- and/or chondroitin-compound complex, thereby delivering the HA-, chondroitin sulfate- and/or chondroitin-compound complex to the cells of such tissue.

The compound-HA, compound-chondroitin or compound-chondroitin sulfate conjugate can be targeted to tissues such as lymph node, bone marrow and liver to minimize the chance of metastasis during surgery to remove a primary tumor. The compound-HA, compound-chondroitin or compound-chondroitin sulfate conjugate can also be administered and directed to HARE in such tissues after there is evidence for metastasis.

A treatment protocol that could be utilized in such a method includes a specific drug, whether a peptide or other chemical or compound, conjugated to HA, chondroitin sulfate and/or chondroitin and used at a dose in the range of from about 5 mg to about 300 mg taken daily and administered either by intravenous injection, by subcutaneous injection or by use of an automated delivery device such as a time release skin patch or a small implanted pump, such as used for delivery of insulin.

In one embodiment, the presently disclosed and claimed invention provides a method for preventing, in a subject, a disease or condition associated with an aberrant HARE expression or activity, by administering to the subject an agent that modulates HARE expression or at least one HARE activity. Subjects at risk for a disease that is caused or contributed to by aberrant HARE expression or activity can be identified by, for example, any or a combination of diagnostic or prognostic assays as described herein. Administration of a prophylactic agent can occur prior to the manifestation of symptoms characteristic of the HARE aberrancy, such that a disease or disorder is prevented or, alternatively, delayed in its progression. Depending upon the type of HARE aberrancy, for example, a HARE agonist or HARE antagonist agent can be used for treating the subject. Such prophylactic methods are discussed in more detail hereinbelow.

Other methods envisioned by the present invention involve methods of treating a disease in a patient wherein one symptom of the disease is an elevated level of at least one of HA, chondroitin and chondroitin sulfate in the blood or lymph. In one embodiment, the method comprises administering to a patient an effective amount of a plasmid, cosmid, phage, viral vector or other vector encoding a functionally active HARE or a functionally active variant or fragment of HARE. The vector should be targeted to a specific cell type such that upon transfection or transduction of such cell with such vector, the cell expresses increased levels of HARE (or a variant or fragment thereof) on the surface thereof. This allows such cell to endocytose greater amounts of HA, chondroitin and chondroitin sulfate and thereby clear an increased amount of HA, chondroitin or chondroitin sulfate from the circulation. Preferably, the vector is targeted to a cell that normally expresses HARE and endocytoses HA, chondroitin or chondroitin sulfate, such as but not limited to, reticuloendothelial cells of the liver and the lymphatic system.

In another embodiment, an affinity matrix is formed which comprises a functionally active fragment or variant of HARE bound to a solid support. Through the process of dialysis, the patient's blood or plasma may be exposed to the affinity matrix such that excess HA, chondroitin or chondroitin sulfate in the patient's blood or plasma binds to the functionally active fragment or variant of HARE of the affinity matrix and is thereby removed from the patient's blood or plasma.

In yet another embodiment, an “artificial organ” is created by expressing the HARE gene in compatible cells, which could preferably be the patient's own cells, and using these cells either in culture in vitro or reinfused back into the patient in vivo to clear HA, chondroitin and/or chondroitin sulfate from blood or plasma. The HARE gene may encode full length HARE or a fragment or variant thereof.

A treatment protocol that could be utilized in such a method includes the isolation under sterile conditions of the patient's white blood cells and their exposure, by transfection, transduction or other appropriate method, to a plasmid, cosmid, phage, viral vector or other vector encoding a functionally active HARE (or active fragment or variant thereof) such that the recipient cells then express an active HARE (or active fragment or variant thereof) capable of binding and internalizing HA, chondroitin sulfate and/or chondroitin from the surrounding milieu. The patient's cells are then transfused back into the patient wherein these cells containing HARE (or a fragment or variant thereof) are then able to lower the blood concentration of HA, chondroitin sulfate and/or chondroitin as desired.

In a further embodiment of the present invention, a soluble fragment or variant of HARE that retains the ability to specifically bind at least one of HA, chondroitin and chondroitin sulfate is utilized to detect HA, chondroitin or chondroitin sulfate in a variety of applications, including ELISA assays and immunocytochemistry. Such soluble fragment or variant of HARE may include an extracellular domain of HARE or an HA-binding domain, a chondroitin-binding domain or a chondroitin-sulfate binding domain of HARE. Clinically, the soluble fragment or variant of HARE could be used to make a test kit for measurement of levels of HA, chondroitin and/or chondroitin sulfate in bodily fluids such as but not limited to, urine, blood, tears, saliva and sweat, such information as may be needed for diagnostic procedures, particularly those related to diseases and cancers that are accompanied by significant elevations of the circulating levels of HA, chondroitin and/or chondroitin sulfate.

For example, the HARE fragment or variant may comprise only an HA-binding domain of HARE and not a chondroitin-binding domain or a chondroitin sulfate-binding domain, and therefore has a unique, predetermined specificity for HA binding and not chondroitin or chondroitin sulfate binding. In this instance, a protocol that could be utilized in such a method includes immobilizing the HARE-derived protein domain (containing an HA-binding domain) on a solid support by methods known to those in the art, such as by covalent attachment of a HARE-derived protein domain to a bead support, such as CNBr-activated Sepharose, and establishment of a negative competition binding assay in which a radiolabeled, biotinylated, fluorescently labeled or otherwise suitably tagged preparation of HA is allowed to bind to the solid HARE-containing support in the absence and presence of increasing amounts of the liquid sample to be tested. Based on a standard curve with known amounts of nonlabeled HA, the amount of HA present in the sample can be calculated.

In another example, the HARE fragment or variant may comprise only a chondroitin-binding domain of HARE and not an HA-binding domain or a chondroitin sulfate-binding domain, and therefore has a unique, predetermined specificity for chondroitin binding and not HA or chondroitin sulfate binding. In this instance, a protocol that could be utilized in such a method includes immobilizing the HARE-derived protein domain (containing a chondroitin-binding domain) on a solid support by methods known to those in the art, such as by covalent attachment of the HARE-derived protein domain to a bead support, such as CNBr-activated Sepharose, and establishment of a negative competition binding assay in which a radiolabeled, biotinylated, fluorescently labeled or otherwise suitably tagged preparation of chondroitin is allowed to bind to the solid HARE-containing support in the absence and presence of increasing amounts of the liquid sample to be tested. Based on a standard curve with known amounts of nonlabeled chondroitin, the amount of chondroitin present in the sample can be calculated.

In yet another example, the HARE fragment or variant may comprise only a chondroitin sulfate-binding domain of HARE and not a HA-binding domain or a chondroitin-binding domain, and therefore has a unique, predetermined specificity for chondroitin sulfate binding and not HA or chondroitin binding. In this instance, a protocol that could be utilized in such a method includes immobilizing the HARE-derived protein domain (containing a chondroitin sulfate-binding domain) on a solid support by methods known to those in the art, such as by covalent attachment of the HARE-derived protein domain to a bead support, such as CNBr-activated Sepharose, and establishment of a negative competition binding assay in which a radiolabeled, biotinylated, fluorescently labeled or otherwise suitably tagged preparation of chondroitin sulfate is allowed to bind to the solid HARE-containing support in the absence and presence of increasing amounts of the liquid sample to be tested. Based on a standard curve with known amounts of nonlabeled chondroitin sulfate, the amount of chondroitin sulfate present in the sample can be calculated.

In a further example, the HARE fragment or variant may comprise two or more of the HA-binding domain, chondroitin-binding domain and chondroitin sulfate-binding domain of HARE. In this instance, a protocol that could be utilized in such a method includes immobilizing the HARE-derived protein domains on a solid support by methods known to those in the art, such as by covalent attachment of the HARE-derived protein domains to a bead support, such as CNBr-activated Sepharose, and establishment of a negative competition binding assay in which a radiolabeled, biotinylated, fluorescently labeled or otherwise suitably tagged preparation of at least one of HA, chondroitin and chondroitin sulfate is allowed to bind to the solid HARE-containing support in the absence and presence of increasing amounts of the liquid sample to be tested. Based on a standard curve with known amounts of at least one of nonlabeled HA, nonlabeled chondroitin and non-labeled chondroitin sulfate, the amount of HA, chondroitin and chondroitin sulfate present in the sample can be calculated. If desired, identification of the particular glycosaminoglycan present among HA, chondroitin sulfate or chondroitin can be further elucidated by utilizing treatment of the sample with specific glycosidases to differentiate the various contributions to the overall assay result by each of either HA, chondroitin sulfate or chondroitin, and the amount of HA, chondroitin and/or chondroitin sulfate in the sample can be quantitated.

In a similar manner as described above for the negative competition binding assay, one can also develop a capture assay for measuring levels of HA, chondroitin or chondroitin sulfate in a sample, such as a biological fluid. A HARE fragment or variant, such as an HA, chondroitin and/or chondroitin sulfate binding region of HARE, is immobilized by attachment to a solid phase. A sample is contacted with the immobilized fragment, thereby allowing HA, chondroitin or chondroitin sulfate present in the sample to bind to the immobilized HARE protein or peptide fragment or variant. The sample is then washed away, and a suitably labeled HARE protein or fragment or variant thereof (or labeled HARE peptide containing the HA, chondroitin and/or chondroitin sulfate binding domains) is used to detect HA, chondroitin or chondroitin sulfate bound to the immobilized HARE protein or peptide fragment or variant.

It is to be understood that test kits for measurements of HA, chondroitin and/or chondroitin sulfate in a sample utilizing the negative competition assay or the capture assay both fall within the scope of the present invention. A test kit which could be utilized for detecting HA, chondroitin and/or chondroitin sulfate by the negative competition assay comprises an immobilized HARE protein or an immobilized HARE peptide fragment or variant that contains HA, chondroitin and/or chondroitin sulfate binding domains, a labeled or tagged preparation of HA, chondroitin and/or chondroitin sulfate, means for contacting the sample with a portion of the immobilized HARE protein or peptide fragment or variant to form a mixture thereof, and means for contacting the labeled or tagged preparation of HA, chondroitin and/or chondroitin sulfate with immobilized HARE protein or peptide fragment or variant alone and with the mixture of sample and immobilized HARE protein or peptide fragment or variant. The kit may further include a known amount of nonlabeled HA for preparing a standard curve for calculating the amount of HA, chondroitin or chondroitin sulfate present in the sample. In addition, the kit may also further include at least one specific glycosidase for identifying the particular glycosaminoglycans present among HA, chondroitin and chondroitin sulfate in the sample.

A test kit which could be utilized for detecting HA, chondroitin and/or chondroitin sulfate by the capture assay comprises an immobilized HARE protein or an immobilized HARE peptide fragment or variant that contains HA, chondroitin and/or chondroitin sulfate binding domains, a labeled or tagged preparation of HARE protein or HARE peptide fragment or variant that contains HA, chondroitin and/or chondroitin sulfate binding domains, means for contacting the sample with a portion of the immobilized HARE protein or peptide fragment or variant to form a mixture thereof, means for washing away unbound sample, and means for contacting the labeled or tagged preparation of HARE protein or peptide fragment or variant with HA, chondroitin and/or chondroitin sulfate (present in the sample) bound to the immobilized HARE protein or peptide fragment or variant. In addition, the kit may further include at least one specific glycosidase for identifying the particular glycosaminoglycans present among HA, chondroitin and chondroitin sulfate in the sample.

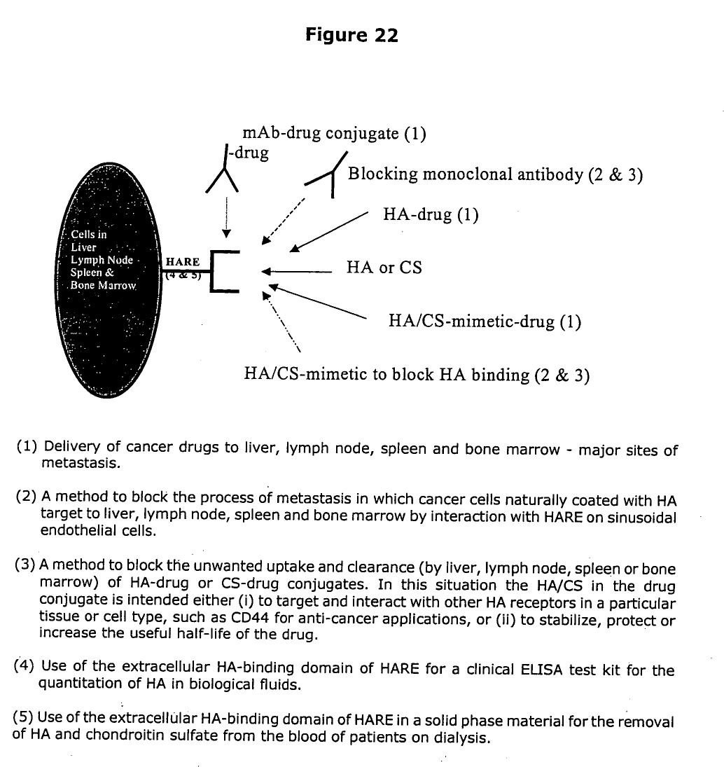

FIG. 22 provides a schematic illustration of some of the above-described methods of the present invention.

The following examples illustrate the practice of the preferred embodiments of the present invention. However, the present invention is not limited to the examples set forth.

### EXAMPLE

U.S. Ser. Nos. 09/842,930 and 10/133,172, which have previously been incorporated herein by reference, disclose the identification and characterization of functionally active Hyaluronan Receptor for Endocytosis (HARE) from rat liver which is able to specifically bind at least one of HA, chondroitin and chondroitin sulfate and endocytose the bound HA, chondroitin or chondroitin sulfate into a cell via a clathrin-coated pit pathway, as well as the purification of 190 kDa and 315 kDa human HARE and the identification and assembly of a human cDNA sequence encoding the 190 kDa HARE. U.S. Ser. Nos. 09/842,930 and 10/133,172 also disclose the isolation of monoclonal antibodies raised against an HA-binding domain of rat HARE, wherein at least one of the monoclonal antibodies blocks binding of HA to HARE. FIGS. 1-15 and 18-25 are provided herein to summarize the identification of the rat liver and human spleen HAREs and the characterization of the rat HARE as well as the isolation of such monoclonal antibodies against the HA-binding domain of rat HARE.

### Description of FIGS. 1-11

U.S. Ser. No. 09/842,930 describes the isolation and characterization of two rat liver HARE isoreceptors that are present in liver, spleen and lymph node. The 175 kDa and 300 kDa HARE species are independent isoreceptors, and the 175 kDa HARE is a bone fide endocytic receptor for HA that is capable of functioning independently of the 300 kDa HARE.

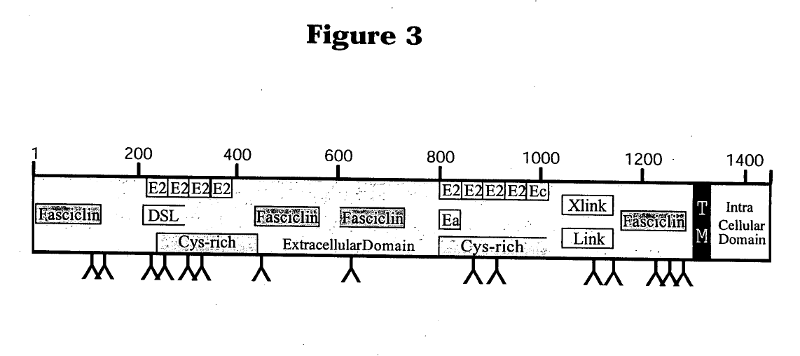

FIG. 1 illustrates the cDNA sequence (SEQ ID NO:1) of the deduced 175 kDa HARE, which encodes a 1431 amino acid protein (SEQ ID NO:2). The protein is predicted to be a type I membrane protein (FIG. 3), with a large NH₂-terminal extracellular domain (1322-1324 residues depending on the particular prediction program used), a single transmembrane domain (˜L¹³²³-A¹³⁴³), and a small COOH-terminal cytoplasmic domain (˜88 amino acids). As is the case for many proteins, the exact boundaries predicted for the transmembrane domain of HARE are somewhat uncertain; they vary by 2-3 amino acids on both sides of the predicted domain depending on the particular algorithm used. For example, the programs TMPred, TMHMM and PSORTII, respectively, predict a transmembrane domain between residues 1327-1347, 1325-1347 and 1327-1343. The predicted mass of the protein is 156,002 Da, and the predicted isoelectric point is pH 7.49. The ectodomain contains 15 putative N-glycosylation sites (excluding one NPS sequon), and two cysteine-rich regions. The extracellular domain has multiple motifs and subdomains with homology to similar regions identified in other receptors and matrix molecules. Multiple EGF-like, βIgH3, and Fasciclin domains, as well as one DSL domain, are also organized throughout the extracellular domain of the 175 kDa HARE. In addition, a 93 amino acid region near the membrane junction (Gly¹⁰⁶³-Arg¹¹⁵⁶) is homologous to the mammalian proteoglycan extracellular Xlink domain and the HA-binding domain of the link protein.

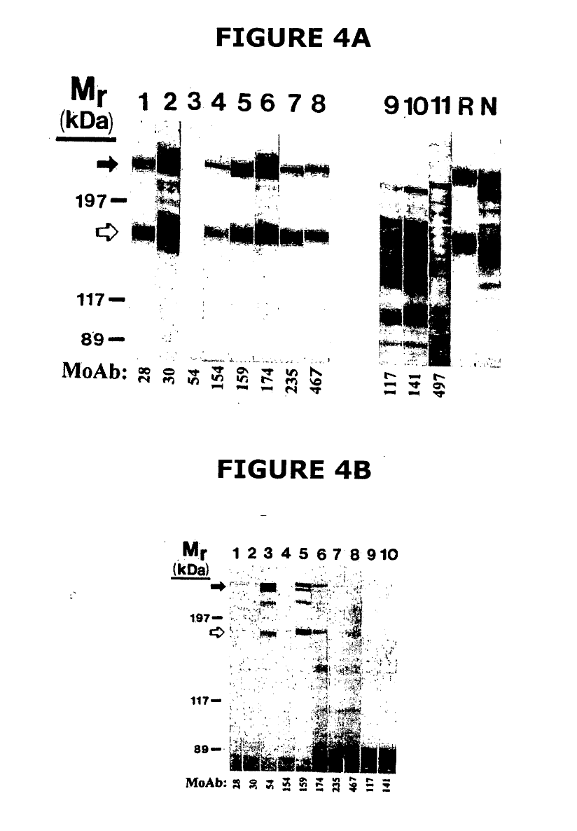

Antibodies were raised utilizing a partially purified fragment of the 175 kDa rat HARE as the antigen, and eleven original monoclonal antibodies were selected as candidates. Eight of the 11 mAbs recognize both the rat LEC 175HARE and 300HARE in Western blots after either nonreducing (FIG. 4A) or reducing (FIG. 4B) SDS-PAGE (mAb's 117, 141 and 497 were not against 175HARE, since they have a different Western pattern and do not immunoprecipitate HARE). Three mAbs (numbers 54, 159 and 174) recognize both reduced HAREs in Western blots. Most of the mAbs raised against the nonreduced 175HARE no longer react with either HARE species after reduction (FIGS. 4A and 4B). The exceptions are mAb-159 and mAb-174, which recognize both the 175HARE and 300HARE proteins in Western blots, whether they are reduced (FIG. 4B) or nonreduced (FIG. 4B). MAb-54 recognizes only the reduced HAREs (FIGS. 4A and 4B, lanes 3).

Four of the mAbs also immunoprecipitate both proteins from LEC extracts. Surprisingly, all mAbs that bind to the 175HARE species, the original antigen, also recognize the 300HARE species. However, as described below, the 300 kDa species is not a dimer of the 175 kDa protein and does not contain a 175 kDa subunit. That eight of eight mAbs raised against the 175HARE cross-react with the 300HARE suggests that the two proteins share one or more common epitopes that may be very antigenic. Except for mAb-159 (IgM) and mAb-30 (IgG_{2b}), all of the HARE-specific mAbs are IgG₁. Listed in Table II are the characteristics of the eight mAbs raised against the rat 175HARE.

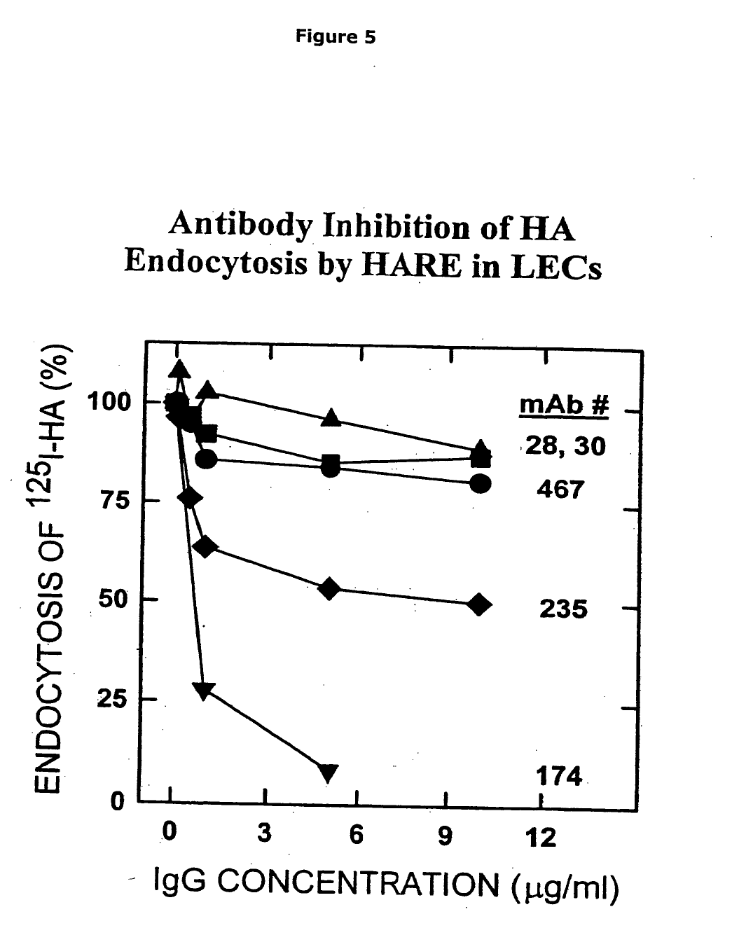

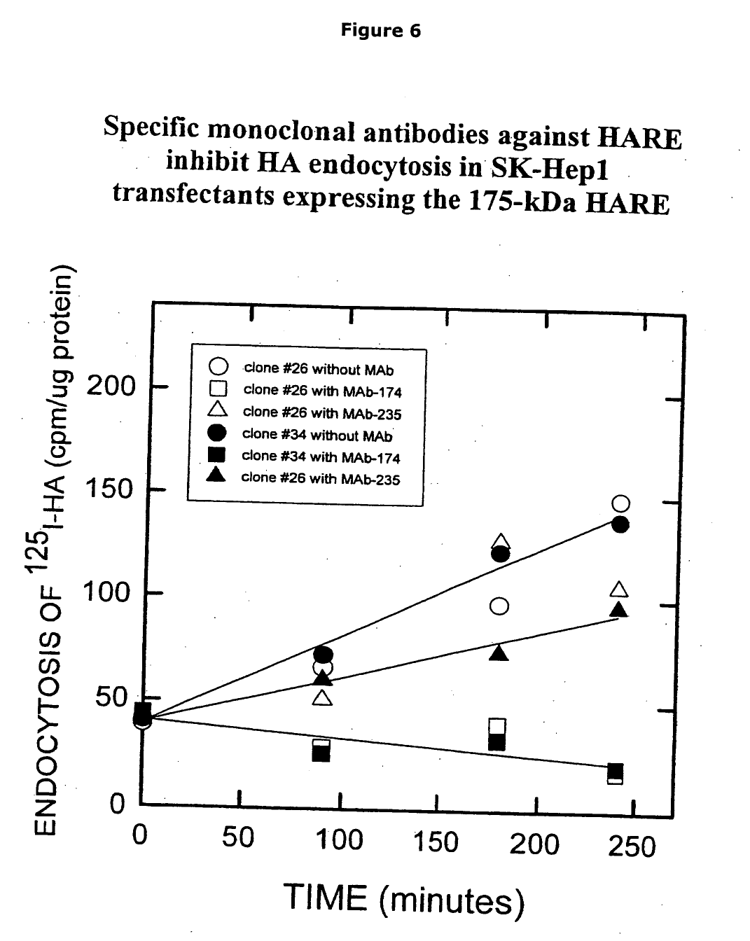

FIGS. 5 and 6 illustrate the specificity of monoclonal antibodies raised against the rat liver 175 kDa HARE protein. Endocytosis and accumulation of ¹²⁵I-HA at 37° C. by cultured LECs was completely inhibited by MAb-174 (FIG. 5). Only one other MAb (#235) had any appreciable affect on HA endocytosis, consistently causing partial (about 50%) inhibition of ¹²⁵I-HA endocytosis. The same results were seen with a SK-Hep1 cell line transfected with cDNA encoding a recombinant 175-kDa HARE (FIG. 6).

Western blot analysis and confocal indirect immunofluorescence demonstrated that the HARE proteins are expressed in spleen as well as in liver, but are not present or are present at much lower levels in brain, lung, heart, muscle, kidney and intestine. The HARE proteins are localized to the sinusoids in the liver and were not observed in parenchymal cells. In addition, the protein is not expressed in isolated hepatocytes in culture but is strongly expressed in purified, cultured LECs, in a pattern typical for an endocytic, recycling receptor: at the cell surface, in pericellular vesicles (presumably endosomes), ER and Golgi. In rat spleen, the HARE proteins are present in the venous sinuses of the red pulp, and were not observed in the germinal centers or white pulp of the splenic nodules. In rat lymph nodes, HARE is localized to the medullary sinuses and is not present in the spheroid nodules or their germinal centers.

Three of the monoclonal antibodies raised against the rat 175 kDa HARE (numbers 30, 154 and 159) were able to recognize a human HARE homologue in human spleen. As observed with the rat HARE, two high molecular weight protein species, at ˜190 kDa and ˜315 kDa, were reactive with the mAbs are were able to bind HA. The specific reactivity of the human HARE proteins with mAb-30, which had been used to purify the rat liver HARE, enabled the purification of the HARE proteins directly from detergent extracts of human spleen by immunoaffinity chromatography. The ˜315 kDa HARE is consistently more abundant than the 190 kDa HARE in human spleen. The apparent molar ratio of the ˜315 kDa HARE: 190 kDa HARE in spleen is ˜2-3:1. Interestingly, essentially the reverse ratio is observed for the two HARE isoreceptors in rat liver.

Upon subunit characterization of the two human HARE isoreceptors, it was determined that the 190 kDa HARE contains only one polypeptide, which migrates at ˜196 kDa after reduction. The ˜315 kDa HARE contains at least two types of disulfide-bonded subunits, which migrate at ˜220 kDa and ˜250 kDa upon reduction. The apparent molar ratio of 250 kDa:220 kDa subunits is about 2-3:1. In contrast, the rat 300 kDa HARE contains three subunits of 97, 230 and 260 kDa in apparent molar ratios of 1:1:1, respectively.

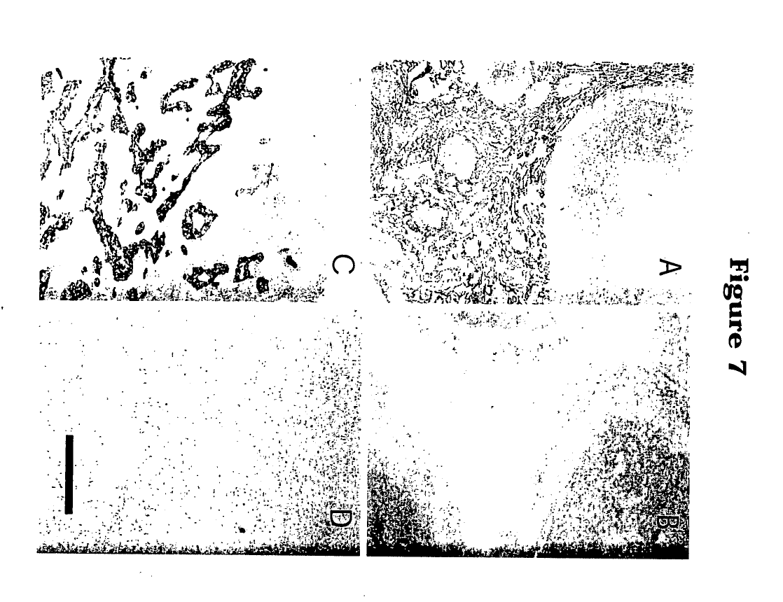

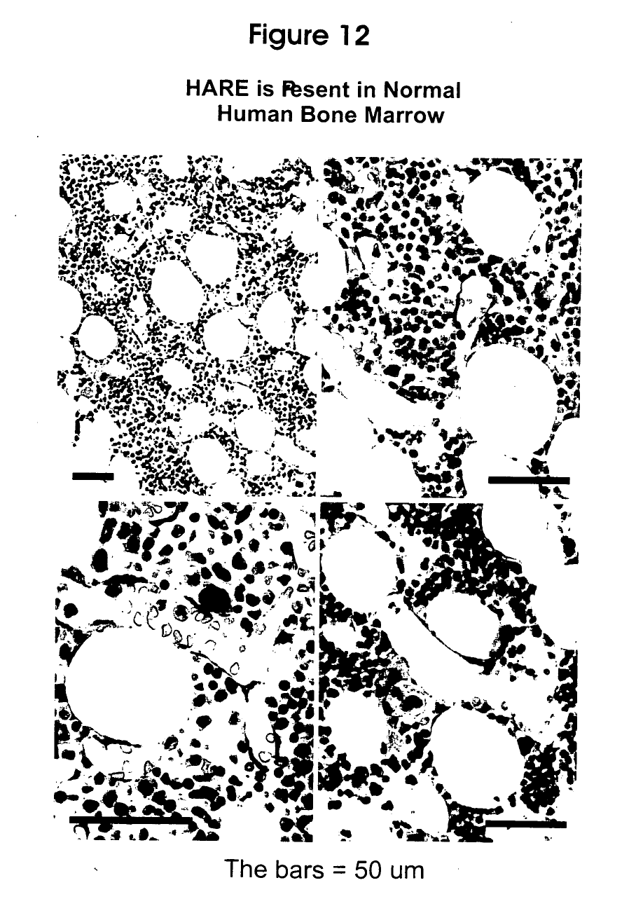

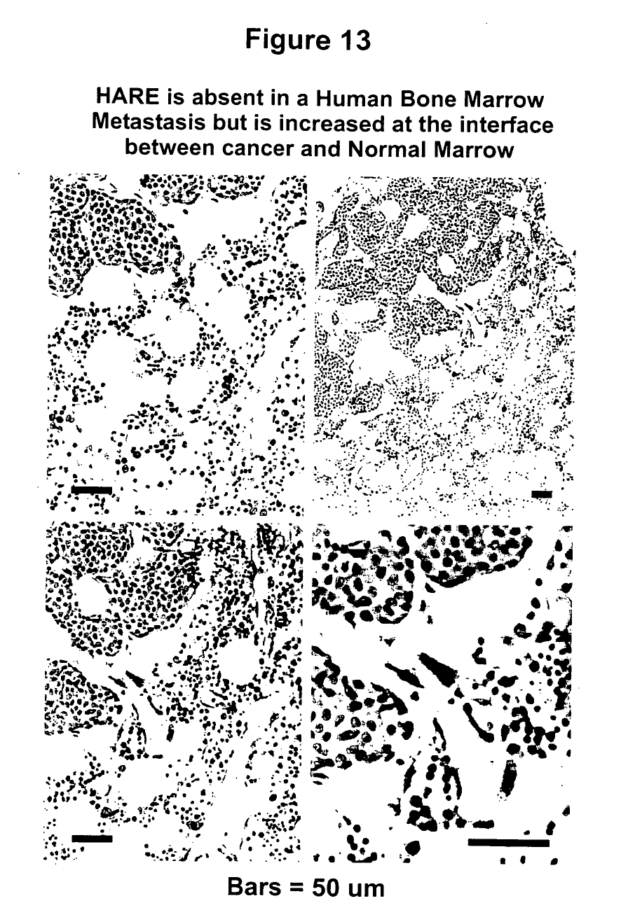

Using mAb-30, abundant HARE protein expression was found in human liver, spleen and lymph node (FIG. 7) and in bone marrow (FIGS. 12 and 13). Staining intensity, and therefore protein expression levels, were much greater in lymph node than in spleen than in liver. In each tissue, only cells in the sinusoidal regions were stained. In spleen, the germinal centers and white pulp areas of spleenic nodules were unstained, whereas the venous sinusoids of the red pulp stained strongly. A more thorough examination of other human tissues is still in progress.

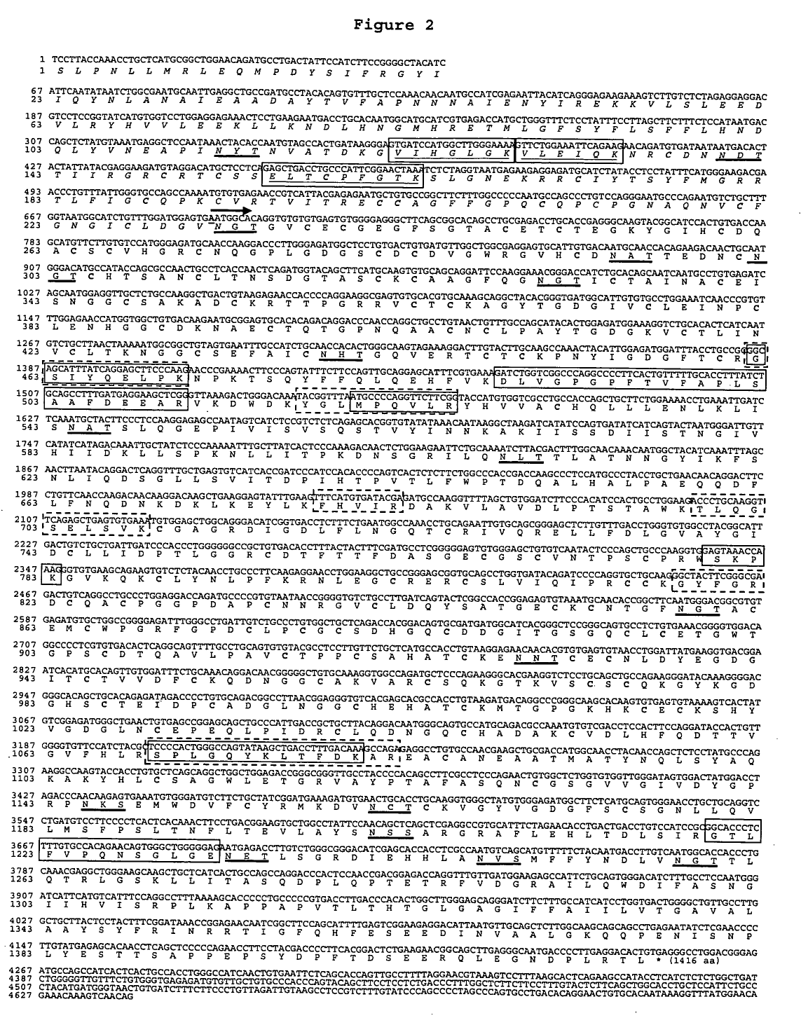

The nucleic acid sequence (SEQ ID NO:3) and deduced protein sequence (SEQ ID NO:4) for the 190 kDa human HARE are shown in FIG. 2. The BAB15793 nucleotide sequence contains a partial ORF of 1193 amino acids that starts at nucleotide position 606. The RT-PCR products generated from spleen mRNA confirmed almost all of the 4575 bp BAB15793 sequence with several important exceptions. Most significantly, key results characterizing new human HARE sequences were obtained from the most 5′ PCR product that was derived from an upstream region of BAB15793 that had been incorrectly concluded to be untranslated. The majority of this 418 bp PCR product is upstream of the putative Trp residue that begins the BAB15793 hypothetical protein sequence (FIG. 2). In fact, the first seven residues of this hypothetical sequence were incorrect due to a frameshift error. Other PCR products are in-frame with, and extend the size of, the human HARE ORF to at least 4251 bp, ending at a stop codon and encoding a protein of 1416 residues. This additional deduced protein sequence contains another three tryptic peptides identified from

TABLE II
Characteristics of mAbs against the rat and human HARE isoreceptors

| | Mouse Monoclonal Antibody Number | | | | | | | |
|---|---|---|---|---|---|---|---|---|
| Property | 28 | 30 | 54 | 154 | 159 | 174 | 235 | 467 |
| Immunoprecipitation of the rat 175 kDa | + | + | − | − | + | + | + | + |
| HARE | | | | | | | | |
| Immunoprecipitation of the rat 300 kDa | + | + | − | − | + | + | + | + |
| HARE | | | | | | | | |
| Recognizes nonreduced rat 175 kDa | + | + | − | + | + | + | + | + |
| HARE in WB | | | | | | | | |
| Recognizes nonreduced rat 300 kDa | + | + | − | + | + | + | + | + |
| HARE in WB | | | | | | | | |
| Recognizes reduced rat 175 kDa HARE | − | − | + | − | + | ˜ + | − | − |
| in WB | | | | | | | | |
| Recognizes 260 kDa subunit of rat | − | − | + | − | + | ˜ + | − | − |
| 300 kDa HARE in WB | | | | | | | | |
| Recognizes 230 kDa subunit of rat | − | − | + | − | + | ˜ + | − | − |
| 300 kDa HARE in WB | | | | | | | | |
| Recognizes 97 kDa subunit of rat | − | − | − | − | − | − | − | − |
| 300 kDa HARE in WB | | | | | | | | |
| Blocks HA uptake in rat LECs at 37- | − | − | − | − | − | + | + | − |
| degrees | | | | | | | | |
| Blocks HA binding to 175 kDa HARE in | − | − | − | − | − | + | − | − |
| blots | | | | | | | | |
| Blocks HA binding to 300 kDa HARE in | − | − | − | − | − | + | − | − |
| blots | | | | | | | | |
| Immunocytochemistry of rat tissues | + | + | + | + | + | + | + | + |
| Immunoprecipitation of the human 190 | − | + | − | − | − | − | − | − |
| kDa HARE | | | | | | | | |
| Immunoprecipitation of the human 315 | − | + | − | − | − | − | − | − |
| kDa HARE | | | | | | | | |
| Recognizes nonreduced human 190 | − | + | − | + | − | − | − | − |
| kDa HARE in WB | | | | | | | | |
| Recognizes nonreduced human 315 | − | + | − | + | − | − | − | − |
| kDa HARE in WB | | | | | | | | |
| Recognizes reduced human 190 kDa | − | − | − | − | + | − | − | − |
| HARE in WB | | | | | | | | |
| Recognizes 250 kDa subunit of human | − | − | − | − | + | − | − | − |
| 315 kDa HARE in WB | | | | | | | | |
| Recognizes 220 kDa subunit of human | − | − | − | − | + | − | − | − |
| 315 kDa HARE in WB | | | | | | | | |
| Immunocytochemistry of human tissues | − | + | − | + | + | − | − | − |

The 8 mAbs raised against the rat liver 175 kDa HARE were tested for their usefulness (+, yes; −, no) as reagents: for immunoprecipitation or Western blot (WB) analysis of either the rat or human small (175-190 kDa) or large (300-315 kDa) HARE proteins; for inhibition of HA binding to LECs or to either HARE in a ligand blot assay; and for immunocytochemical analysis of HARE expression in rat or human tissues.

the purified HARE protein and is 83% identical to the same 139 residue region in the rat 175 kDa HARE.

The entire 1416 amino acid open reading frame (4251 nucleotides) of the human 190 kDa HARE (SEQ ID NO:4) has been successfully amplified from a human lymph node cDNA library. A similar bp PCR product was also seen with a comparable cDNA library prepared from human spleen.

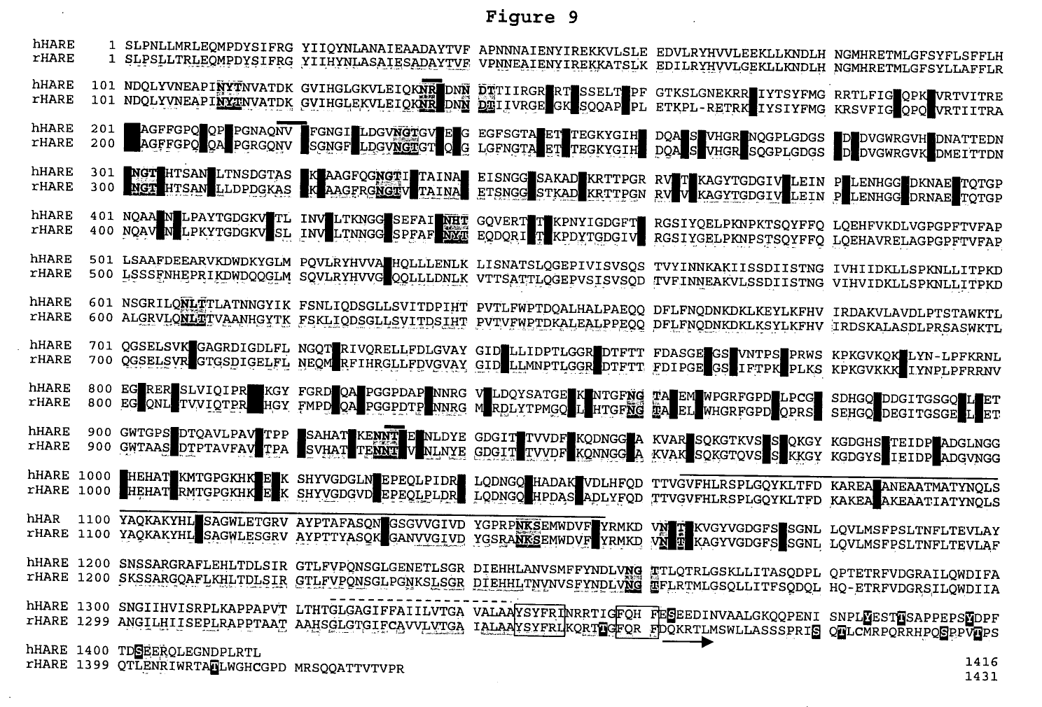

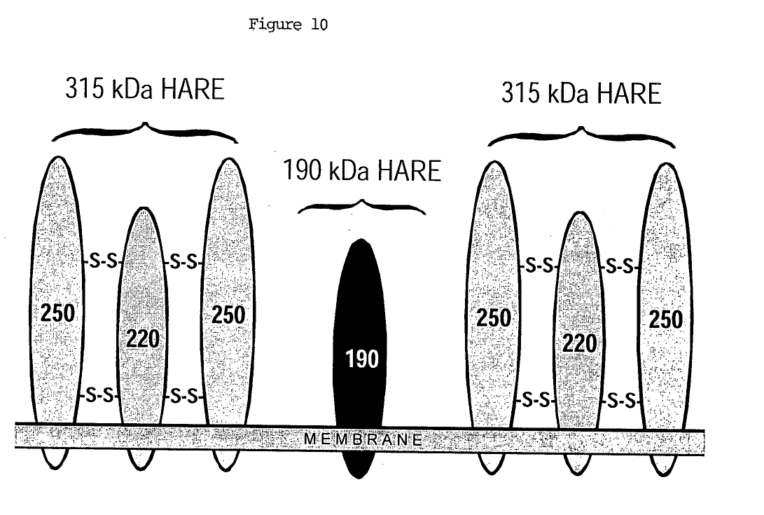

The human partial cDNA encoding the 190 kDa HARE in fact encodes (in its entirety) for a much larger protein which is consistent with the finding for the rat HARE that a large precursor protein gives rise to the smaller HARE. For example, the two largest rat HARE proteins were demonstrated to be reactive with an antibody against a predicted amino acid sequence upstream of the cDNA region encoding the native rat 175-kDa HARE. Furthermore, the partial human cDNA for HARE encodes a protein with almost the identical N-terminal 20-residue sequence found for the rat 175 kDa HARE (FIG. 9). This human core protein for the 190 kDa HARE corresponds with a very high level of identity and similarity to the rat 175 kDa HARE protein. Despite the apparent size difference between the human 190 kDa and rat 175 kDa HARE species, the sizes of the two core proteins are identical. In this experiment, the affinity purified proteins were treated with endoglycosidase F to remove N-linked oligosaccharides and then analyzed by SDS-PAGE and Western blotting to detect the human and rat HARE core proteins.

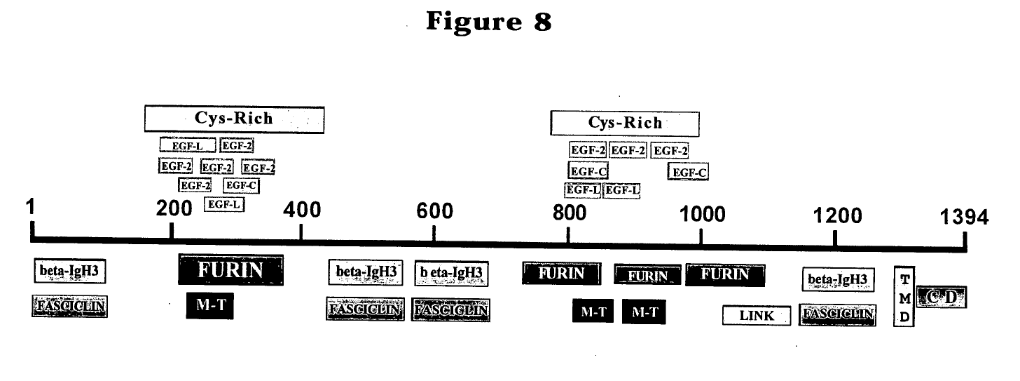

The human HARE is predicted to be a type I membrane protein (FIG. 8), with a large NH₂-terminal extracellular domain (>1300 amino acids), a single transmembrane domain (˜21 amino acids), and a small COOH-terminal cytoplasmic domain (˜72 amino acids). The predicted mass of the 1416 residue partial core protein determined here is 154,091 Da, and the pI is pH 5.91. The protein contains 17 potential N-glycosylation sites (-N-X-T/S-) in the extracellular domain. Twelve of these sites are identical with sites in the rat 175 kDa HARE (FIG. 9). An additional three nonclassical glycosylation sequons (-N-X-C-) are present in the human HARE, two of which are conserved with the rat HARE. An interesting feature of these Cys-containing sites is that glycosylation and participation of the Cys in a disulfide bond may be mutually exclusive (Miletich and Broze, (1990)). The 190 kDa HARE extracellular domain has two cysteine-rich regions and multiple EGF-like, βIgH3, Furin, Metallothionein and Fasciclin domains, as well as DSL domains and one 93 amino acid Link (or XLink) domain near the membrane junction (Gly¹⁰⁶³-Tyr¹¹⁵⁵). Many of the programs such as Pfam-HMM, ScanProsite, SMART (Schultz et al., (1998)) or CD-Search identify domains that are only partial or weak matches and overlap with other domains. In particular the EGF-like domains show this characteristic (FIG. 8). Although the overall organization of all these above domains is very similar between the human and rat HARE proteins, the exact arrangement and number of each type of domain is not identical.

The human 190 kDa HARE and the rat 175 kDa HARE protein sequences are 78.1% identical, with a gap frequency of only 0.2% (using the SIM Alignment Program), over a region containing 1416 residues (FIG. 9). An additional ˜6.5% of the amino acid differences between the two proteins are conservative substitutions (e.g. R/K or S/T). Almost all of the cysteine residues within the extracellular domains of the two HARE proteins are absolutely conserved, which suggests that the two proteins have the same overall folding and organization of their polypeptide chains. Unlike the rat protein, the human HARE has no cysteine residues in its transmembrane or cytoplasmic domains. The cytoplasmic domains of the two HARE proteins are less conserved (˜25% identical) than their transmembrane (˜76% identical) or extracellular domains (˜80% identical). Nonetheless, two candidate φXXB motifs for targeting these receptors to coated pits are highly conserved: the human HARE YSYFRI¹³⁵⁰ and FQHF¹³⁶⁰ motifs differ by only one amino acid from the corresponding regions in the rat HARE cytoplasmic domain (FIG. 9).

Table III identifies several putative motifs from the human HARE protein that may be present in “HARE-like” proteins. Such “HARE-like” proteins have the ability to bind at least one of HA, chondroitin and chondroitin sulfate, and the “HARE-like” proteins comprise the LINK domain (SEQ ID NO:5) and at least one motif selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and sequences that are substantially identical to or only contain conserved or semi-conserved amino acid substitutions to the above-referenced sequences.

### Description of FIGS. 12-25

There is a large literature supporting the involvement of HA itself or hyaluronidases in cancer, particularly in the process of metastasis wherein malignant cells leave a primary tumor, migrate through multiple cell layers to enter and then leave the vasculature and ultimately enter a target tissue where they will establish a secondary tumor. In general the high mortality of cancers is not associated with the primary tumor but rather with the secondary metastases, which are very often found in liver, lymph nodes and bone marrow, the same tissues in which we have disclosed the presence of the HA Receptor for Endocytosis. Auvinen et al., (2000) showed a high correlation between HA expression levels, metastasis to lymph nodes and decreased survival of breast cancer patients. The very close link between metastasis and cellular synthesis of, and interactions with, HA indicates that HA can play a critical role in this process. For example, Simpson et al., (2001)

TABLE III
Putative Motifs of “HARE-like” Proteins

| | | |
|---|---|---|
| SEQ | | |
| ID | | Residues in hHARE |
| NO: | Amino Acid Sequence | (from SEQ ID NO:4) |

| | | |
|---|---|---|
| 5 | GVFHLRSPLGQYKLTFDKAREACANEAAT | G¹⁰⁶³ - Y¹¹⁵⁵ |
| | MATYNQLSYAQKAKYHLCSAGWLETGRVA | |
| | YPTAFASQNCGSGVVGIVDYGPRPNKSEM | |
| | WDVFCY | |
| 6 | GTACETCTEGKYGIHCDQACSCVHGRCNQ | G²⁴⁵ - D²⁹³ |
| | GPLGDGSCDCDVGWRGVHCD | |
| 7 | CKAGYTGDGIVCLEINPCLENHGGCDKNA | C³⁶⁵ - Q⁴⁰² |
| | ECTQTGPNQ | |
| 8 | IDKLLSPKNLLITPKD | I⁵⁸⁵ - D⁶⁰⁰ |
| 9 | ALPAEQQDFLFNQDNKDKLK | A⁶⁵⁴ - K⁶⁷³ |
| 10 | CRIVQRELLFDLGVAYGIDCLLIDPTLGG | C⁷²⁵ - D⁷⁶² |
| | RCDTFTTFD | |
| 11 | DCQACPGGPDAPCNNRGVC | D⁸²³ - C⁸⁴¹ |
| 12 | CKCNTGFNGTACEMCWPGRFGPDC | C⁸⁵¹ - C⁸⁷⁴ |
| 13 | CSDHGQCDDGITGSGQCLCETGWT | C⁸⁷⁹ - T⁹⁰² |
| 14 | YEGDGITCTVVDFC | Y⁹³⁸ - C⁹⁵¹ |
| 15 | GGCAKVARCSQKGTKVSCSC | G⁹⁵⁶ - C⁹⁷⁵ |
| 16 | PCADGLNGGCHEHATC | P⁹⁹¹ - C¹⁰⁰⁶ |
| 17 | TGPGKHKCECKSHYVGDG | T¹⁰⁰⁹ - G¹⁰²⁶ |
| 18 | PIDRCLQDNGQCH | P¹⁰³⁵ - H¹⁰⁴⁷ |

demonstrated that tumor cells producing surface HA are much more able to interact with and bind to bone marrow endothelial cells and that this interaction may be important in the cell homing process by which a malignant prostate cell is able to migrate to and establish itself in bone marrow. Similarly, Itano et al., (1999) showed that mutants of a mouse mammary carcinoma cell line that were unable to synthesize HA had a significantly decreased ability to metastasize in an animal model, but when transfected with a cDNA encoding HA synthase 1, these cells were rescued in their ability to make HA and to metastasize. Other studies support the idea that HA on the tumor cell or the endothelial cell can mediate cell adhesion, which is a critical step in metastasis, if the other cell has a cell surface component able to bind HA (Okada et al., (1999)).

The immunocytochemical localization of human HARE in bone marrow, utilizing our specific monoclonal antibodies against HARE, demonstrates the expression of HARE in the sinusoidal endothelial cells of normal marrow (FIG. 12) in a female patient with primary ductal breast cancer. The control (lower right panel) using mouse serum rather than the anti-HARE mAbs shows no staining. The same patient had metastasis to the femoral head, and FIG. 13 shows that the HARE expression appears normal in regions of marrow adjacent to the cancer (the tumor is to the upper left in all four panels). The cancer cells are not stained for HARE, indicating it is absent in the tumor. In areas immediately adjacent to the cancer, the expression of HARE in the human bone marrow endothelial (HBME) cells appears to be enhanced. The control (upper left panel) using mouse serum rather than the anti-HARE mAbs shows no staining.

HARE mediates HA-dependent adhesion to metastatic prostate and breast cancer cells. FIGS. 12 and 13 show that HARE expression appears enhanced at the interface between normal bone marrow and cancer cells that have metastasized to marrow in a breast cancer patient. Elegant animal studies by Simpson et al. (2001 and 2002) showed that human metastatic tumor cells expressing cell surface HA are targeted to sinusoidal endothelial cells of bone marrow and lymph nodes. To verify that HARE can mediate adhesion to tumor cells expressing HA, cell adhesion studies were performed. It was found that HARE-HA interactions are able to mediate specific cell-cell adhesion between tumor and “normal” cells in vitro. The ability of human cells to adhere via HARE-HA interactions in vitro supports the role of HARE in metastasis of some cancer cells in vivo.

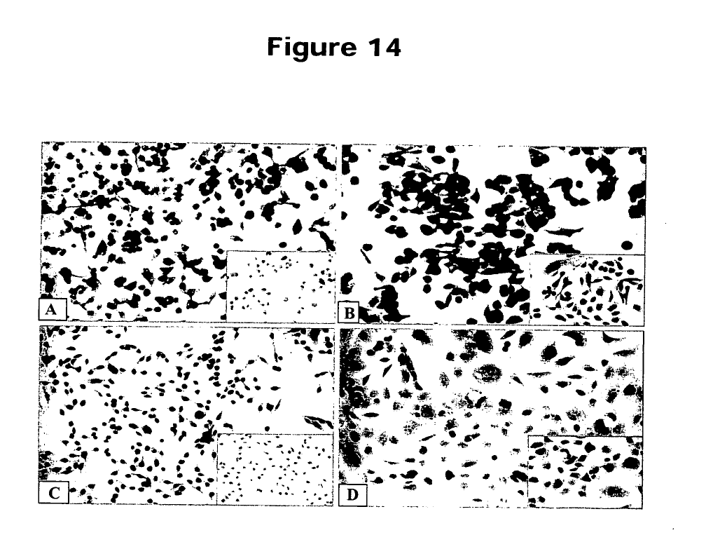

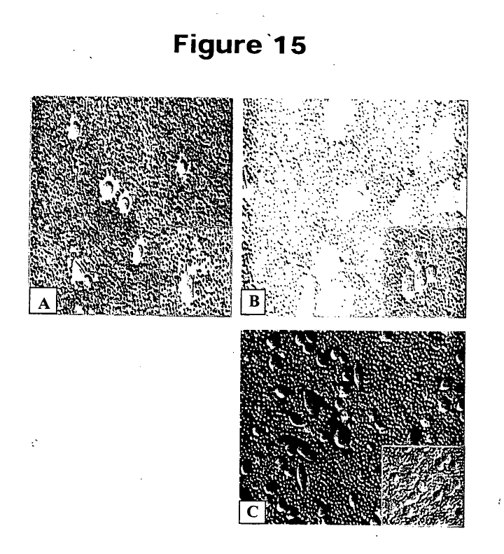

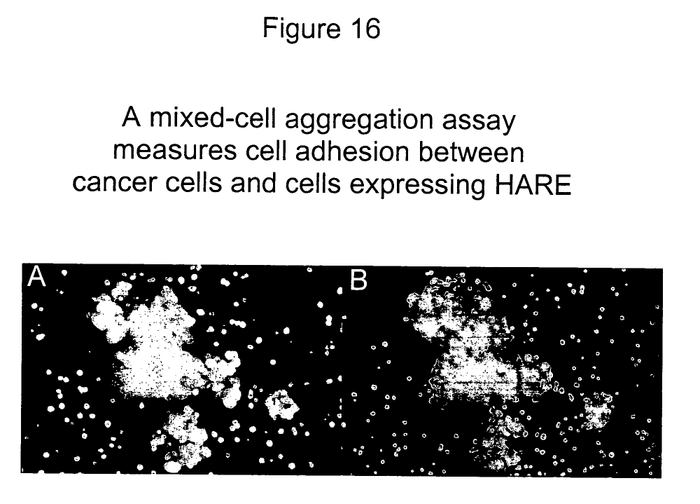

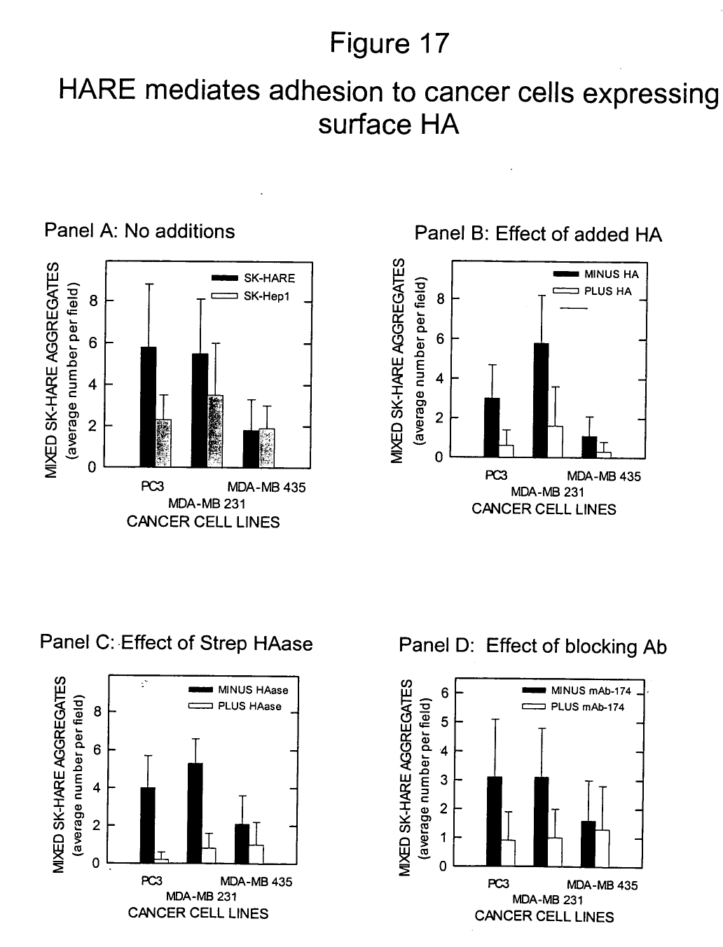

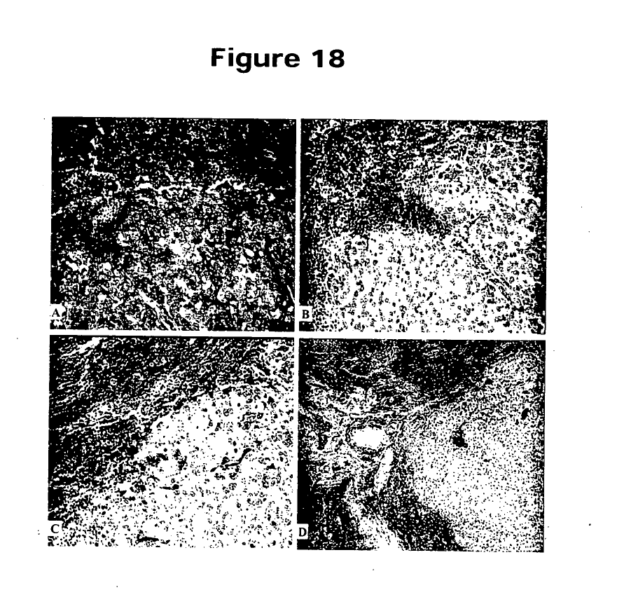

Cell-associated HA has been increasingly associated with carcinoma cell metastasis. Metastasis of some cancer cells to specific tissues could involve specific binding interactions between HA on the tumor cell surface and HA receptors on particular cell types in the target tissue. This possibility was investigated using an in-vitro model of HA mediated carcinoma cell adhesion. The metastatic human breast carcinoma cell line MDA-MB-231 shows increased cell surface HA (based on a particle exclusion assay or staining with a biotinylated-HA binding protein) compared to the metastatic human breast carcinoma cell line MDA-MD435 (FIGS. 14 and 15). Similarly, the human metastatic prostate cancer cell line PC3 has increased peri-cellular HA compared to the less metastatic DU145 human prostate cancer cell line. A mixed cell aggregation assay was used (each cell type was pre-labeled with a different probe) and adhesion was scored as the average number of mixed cell aggregates/field (n=10) using fluorescence microscopy. This semi-quantitative assay does not take into account aggregate size or the percent of each cell type participating in mixed aggregates. Some mixed SK-HARE cell aggregates were quite large, e.g. with PC3 cells (FIG. 16), whereas others were smaller, e.g. with MDA-MB 435 cells (not shown). PC3 and MDA-MB 231 cells adhered much more to the SK-HARE cells compared to the parental SK-Hep-1 cells not expressing HARE (FIG. 17A). In contrast, there was no difference in aggregation of MDA-MB 435 cells (with little HA) to SK-HARE or SK-Hep-1 cells. Strong HARE-dependent adhesion was indicated for the interactions of SK-HARE cells with PC3 and MDA-MB 231 cells because aggregation of each cell pair was strongly blocked by pretreatment with either HA (FIG. 17B), hyaluronidase (FIG. 17C) or the blocking mAb-174 (FIG. 17D). These results support the claim that human cancer cells can adhere to normal cells via interactions between HARE and HA (or another GAG such as a CS) and that this interaction can mediate metastasis, survival or growth of cancer cells in tissues expressing HARE such as liver, lymph node, and bone marrow. HARE, which is highly expressed in liver, lymph node and bone marrow (very common sites of adenocarcinoma metastasis), could be a “homing receptor” that mediates the capture and localization of tumor cells expressing cell surface HA. Tissue sections from lymph nodes containing metastatic breast carcinoma show tumor cells that contain cell surface HA have apparently arrested in the lymph node at sites of HARE expression (FIG. 18).

Carcinoma metastasis requires specific biochemical interactions at the metastatic site between the tumor cells and endothelium to mediate adhesion and tumor cell arrest. In breast carcinoma, subsets of tumor cells undergo phenotype changes allowing them to accomplish all steps in the metastatic cascade. This includes detachment from the primary tumor, invasion of tissue, entry into lymphatics/vasculature, dissemination and avoidance of host defense, arrest at a distant site, exit from the circulation and finally proliferation at the secondary site (Seraj et al., (2000)). Tumor cell arrest in the metastatic site can be facilitated by receptor-ligand interactions. Recent reports indicate that hyaluronan (HA) on prostate carcinoma cell surfaces is important for adhesion of prostate carcinoma cells to bone marrow endothelium (Lehr et al., (1998); Simpson et al., (2001)). The HBME cell surface molecule responsible for this adhesion has not been identified. Candidate HA binding proteins would include CD44 (Simpson et al., (2001)), the Receptor for HA mediated motility (RHAMM) (Lokeswar et al., (2000)), the lymph vessel endothelial specific HA receptor (LYVE-1) (Banerji et al., (1999)) and HARE (Zhou et al., (2000)). Incubation of HBME cells with CD44 blocking antibodies failed to inhibit HA-mediated prostate cancer cell adhesion, making CD44 a less likely candidate (Simpson et al., (2001)). RHAMM has not been described in HBME cells, although it can be involved in lung metastasis (Lokeswar et al., (2000)). LYVE-1 mRNA was detected in bone marrow; however, bone marrow protein expression was not confirmed by immunohistochemistry (Banerji et al., (1999)). HARE is expressed in spleen, liver, lymph node and bone marrow, the latter three organs being common sites of carcinoma metastasis.

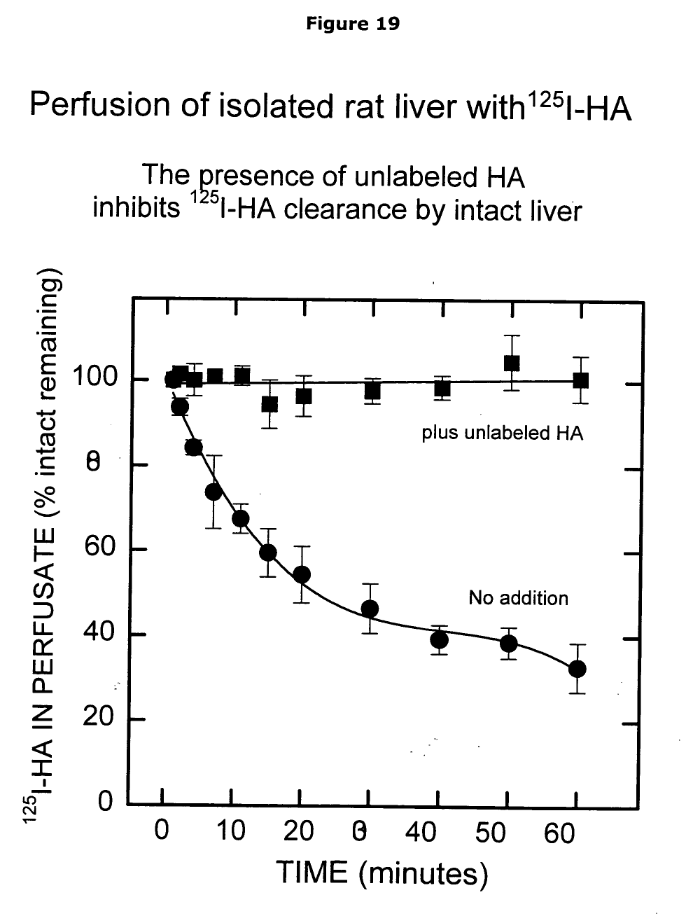

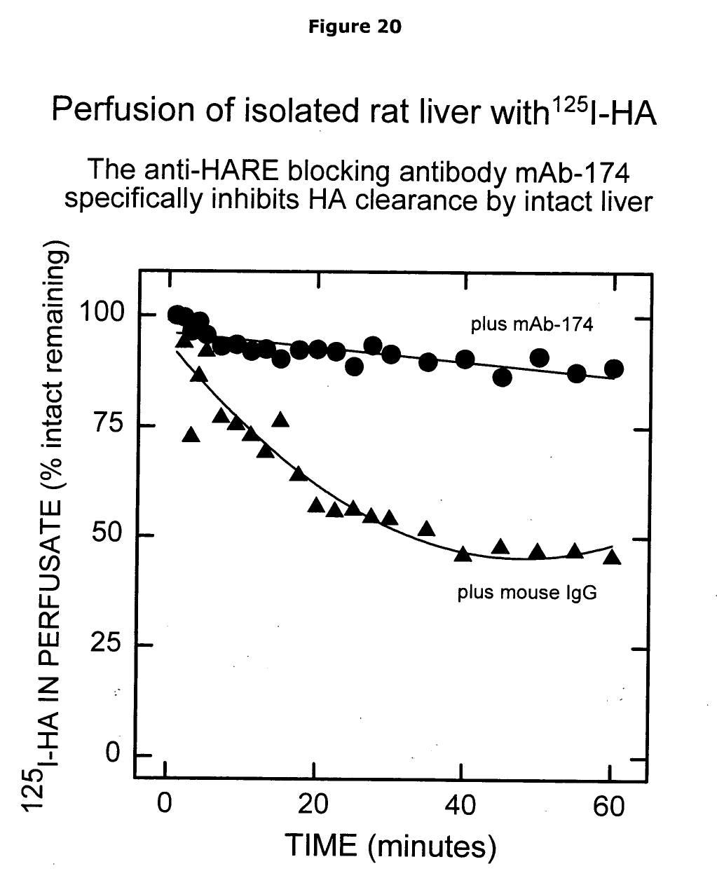

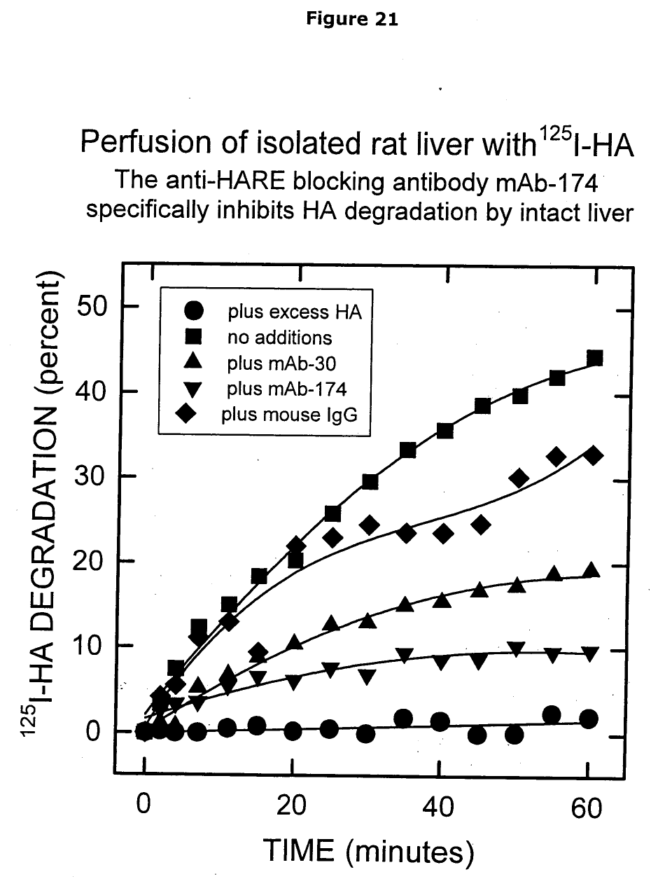

FIGS. 19 and 20 are continuous perfusion (with recirculation) experiments with isolated rat liver that demonstrate that excess unlabeled HA and the anti-HARE blocking antibody mAb-174 specifically inhibit HA clearance by intact liver. FIG. 21 demonstrates that excess unlabeled HA, mAb-30 and mAb-174 specifically inhibit HA degradation by intact liver.

In FIG. 19, isolated rat liver is reperfused with continuous recirculation with ¹²⁵I-HA, and the uptake of ¹²⁵I-HA by the rat liver (labeled as “No addition”) can be observed over time. The addition of unlabeled HA competitively inhibits this uptake, demonstrating that the clearance of ¹²⁵I-HA is due to a receptor that specifically recognizes HA.

In FIG. 20, the anti-HARE blocking antibody mAb-174 also specifically inhibits ¹²⁵I-HA clearance by intact liver, while the addition of mouse IgG does not affect ¹²⁵I-HA uptake by the liver. This demonstrates that the specific receptor responsible for the clearance of ¹²⁵I-HA is HARE. These results are consistent with the findings of Laurent and co-workers that liver is the major site of HA clearance from the blood.

In FIG. 21, isolated rat liver is reperfused with ¹²⁵I-HA, and the degradation of ¹²⁵I-HA by the rat liver (labeled as “no additions”) is observed. The addition of excess HA completely inhibits such degradation, while mAb-30 and mAb-174 also inhibit degradation of ¹²⁵I-HA. The addition of mouse IgG has very little affect of the degradation of ¹²⁵I-HA.

To further confirm that the bone fide cDNA for the rat 175-kDa HARE had been cloned, HA binding and internalization studies were performed using transfected COS-7 or SK-Hep-1 cells expressing the 175-kDa protein. Since there is no natural mRNA directly coding for the 175-kDa HARE protein, an artificial cDNA that encodes the ORF for the 175-kDa HARE fused at the 5′ end to a short region of the Ig k-light chain sequence containing a start codon and a membrane insertion signal or leader sequence was constructed. Transient transfection of this cDNA into COS-7 cells yielded a protein of the expected size that was recognized in Western blots by the specific anti-HARE mAbs and that bound ¹²⁵I-HA specifically in the ligand blot assay.

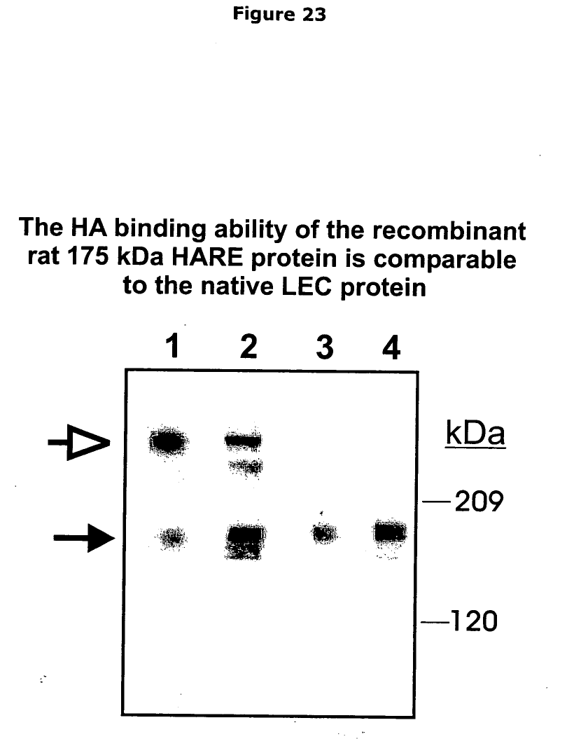

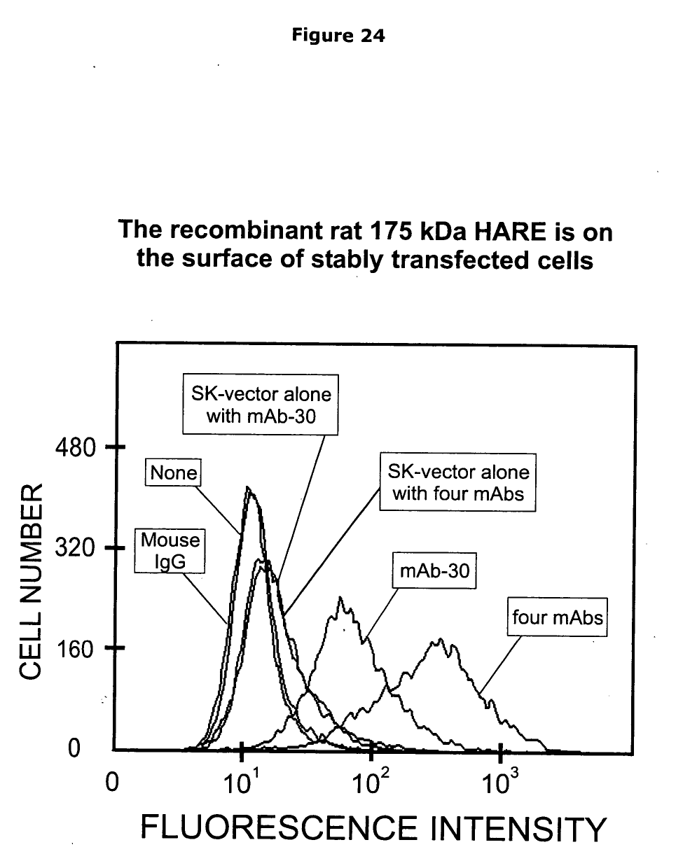

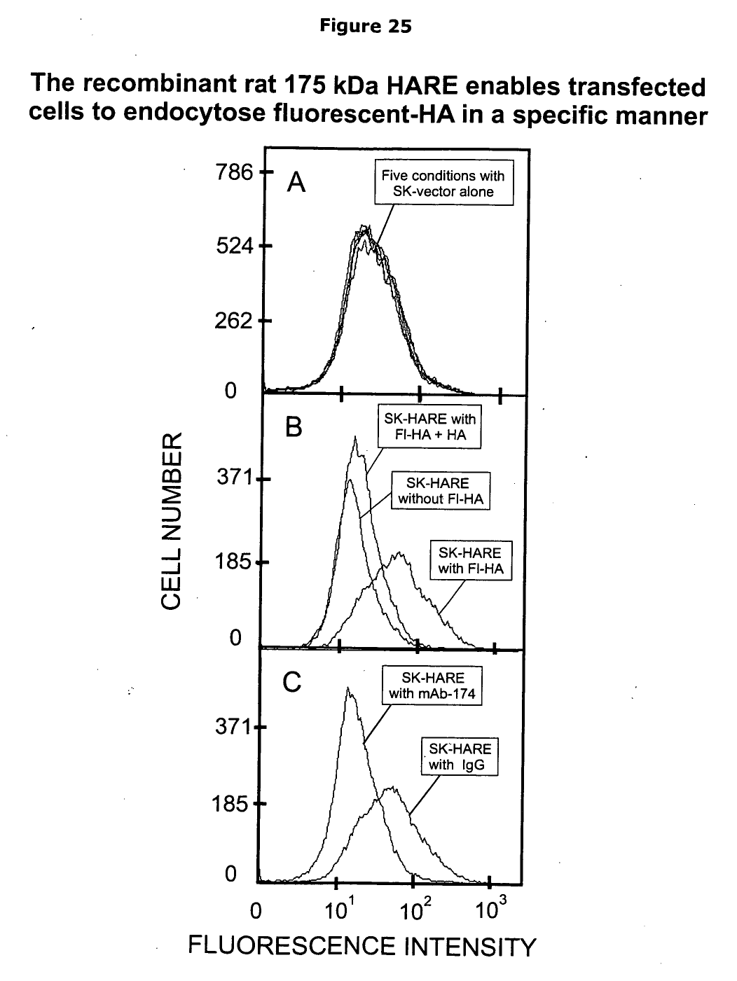

This p175HARE-k vector was then used to generate stable cell lines expressing HARE after antibiotic selection of transfected SK-Hep-1 cells. This cell line was chosen because it does not express any detectable endogenous HA receptors capable of specific ¹²⁵I-HA binding or endocytosis, and does not show reactivity with the anti-HARE mAbs in Western blots. Seven independent clones were selected, all of which had essentially identical characteristics with respect to 175-kDa HARE expression and function. The recombinant 175-kDa HARE expressed by these cells and the purified rat LEC protein were essentially identical in their ability to bind ¹²⁵I-HA in the ligand blot assay (FIG. 23). FACS analysis showed that the recombinant HARE protein was localized to the cell surface (FIG. 24). Specific mAbs against the 175-kDa HARE bound to cells expressing HARE, but not to SK-Hep-1 parental cells or cells transfected with vector alone. The internalization of fluorescent-HA by SK-HARE cells was specific as judged by its competition with unlabeled HA (FIG. 25B), its inhibition by mAb-174 (FIG. 25C), and the lack of uptake by SK-Hep-1 cells or cells transfected with vector alone (FIG. 25A).

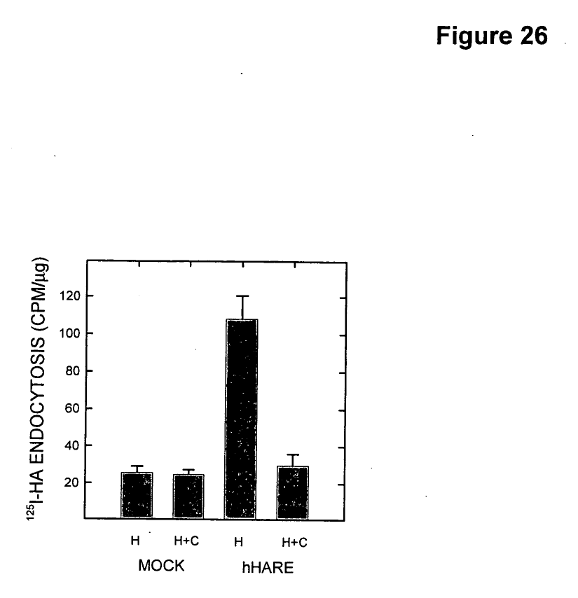

### Description of FIGS. 26-35

The purification of the two hHARE proteins, of 190 kDa and 315 kDa, from spleen extracts, has been described herein previously and in the two parent applications U.S. Ser. Nos. 10/133,172 and 09/842,930, as well as the molecular cloning of partial cDNAs from pooled human lymph node and spleen that encoded part or all of the subunits in these two isoforms. The 190 kDa hHARE protein is not expressed from a unique mRNA, but rather is encoded by a 4383 bp region (1461 amino acids) at the 3′ end of the full-length Stab 2 coding region. In order to express the 190 kDa protein, an artificial cDNA for a recombinant 190 kDa hHARE was created in the pSecTag/FRT/V5/His-TOPO expression vector. For proper membrane orientation and trafficking to the cell surface, the pSecTag vector provides an Ig K-chain secretion signal sequence fused at the N-terminus of the protein. Transiently transfected Flp-In 293 cells expressed sufficiently high levels of the recombinant 190 hHARE to mediate the specific binding and internalization of ¹²⁵I-HA (FIG. 26). Compared to vector alone, cells transfected with hHARE cDNA internalized approximately 4-times the amount of HA and this uptake was completely blocked by unlabeled HA. Specific HA uptake, therefore, was ˜80% of the total.

The potential advantage of using Flp-In 293 cells as the parental cell line for generation of stable cell lines expressing hHARE is that all clones should be virtually identical if the plasmid inserts at only the single unique chromosome site containing the engineered integration site. Correct integration at this site interrupts a β-galactosidase gene and a Zeocin resistance gene in the engineered site. Clones containing a single plasmid insertion at the correct engineered site are, therefore, Hygromycin B resistant, negative for β-galactosidase activity and are Zeocin sensitive. If plasmid insertion occurs at other chromosome sites, rather than the correct engineered site, then clones will express β-galactosidase and be Zeocin resistant. Out of 41 stably transfected clones that we selected and characterized, three (#9, #14, and #40) had no detectable galactosidase activity, were Zeocin sensitive and were judged to contain a plasmid insertion at the unique engineered site.

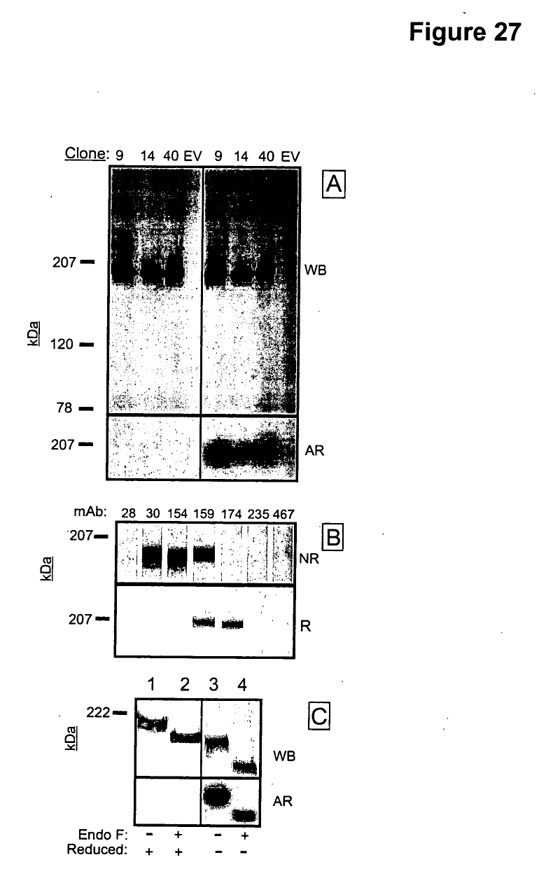

A protein of the correct size for the 190 hHARE was expressed in the three selected stable Flp-In 293 cell lines, and this protein bound ¹²⁵I-HA with >98% specificity in a ligand blot assay following SDS-PAGE and electrotransfer (FIG. 27A). The 190 kDa hHARE protein expressed in Flp-In 293 cells had the characteristics previously found for native hHARE purified from spleen. The recombinant nonreduced protein was recognized in Western blots by the three anti-rHARE mAbs that cross-reacted with native hHARE (mAbs # 30, 154, and 159) but not mAbs # 28, 174, 235 and 467 (FIG. 27B: NR). Similarly, the reduced 190 kDa hHARE protein reacted with only mAbs #159 and 174 (FIG. 27B: R). Based on its HA-binding activity in these in vivo and in vitro assays, the recombinant hHARE protein appeared to be folded properly. Consistent with this interpretation, three other characteristics of the recombinant hHARE were identical to those of the native protein. Reduction of disulfide bonds resulted in slower migration of the 190 hHARE in SDS-PAGE compared to the non-reduced protein (FIG. 27C, lanes 1 and 3; WB). Reduction of disulfide bonds also caused loss of HA-binding activity (FIG. 27C, lanes 1 and 3; AR). After treatment with endoglycosidase-F to release N-linked oligosaccharides, the recombinant protein migrated at a position corresponding to a loss of −25 kDa (FIG. 27C, lanes 3 and 4; WB). The de-N-glycosylated hHARE protein was still able to bind HA in this ligand blot format (FIG. 27C, lane 4; AR). In addition, anti-V5 antibody recognition of the C-terminal epitope provided by the vector, was suitable for immunoprecipitation (not shown).

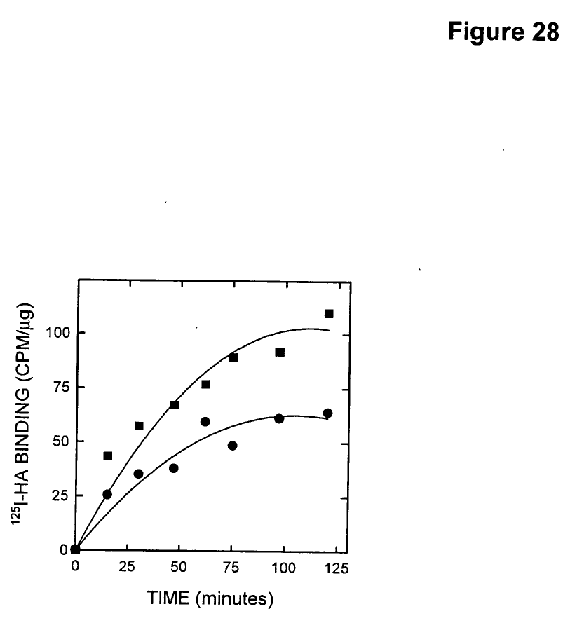

The specific binding of ¹²⁵I-HA at 4° C. by stable cell lines was typical for a membrane bound receptor; binding kinetics was hyperbolic and saturated after about 90 min (FIG. 28). Essentially no specific binding of ¹²⁵I-HA occurred in the control cells transfected with empty vector, consistent with the absence of any significant HA receptor activity in 293 cells (Table IV). Also, as found for other endocytic, recycling receptors (e.g. the asialoglycoprotein and mannose receptors), about 30-50% of the total cellular hHARE population was on the cell surface and the remainder was intracellular. The native rHARE in isolated LECs is an active endocytic receptor that recycles so that HA can be continually internalized and delivered to lysosomes for degradation over many hours (Weigel and Yik, 2002; McGary et al., 1989; McGary et al., 1993). To assess the ability of the recombinant 190 hHARE to recycle, cells were allowed to internalize ¹²⁵I-HA for 4 h, and the amount of specific HA uptake was calculated as the number of cell surface receptor equivalents. This estimates the approximate number of times that a cohort of cell surface HARE proteins would have to be used in order to achieve the observed level of HA uptake. For 190 hHARE Flp-In 293 clones #9 and #14, these recycling ratios were 25 and 32, respectively (Table IV). Based on these values of 25-32 surface equivalents of HA internalized in 240 min, the estimated individual receptor recycling time is 7.5-9.6 min, which is identical to the recycling times reported for all the known coated pit mediated clearance receptors that recycle (Weigel and Yik, 2002; Mellman, 1996).

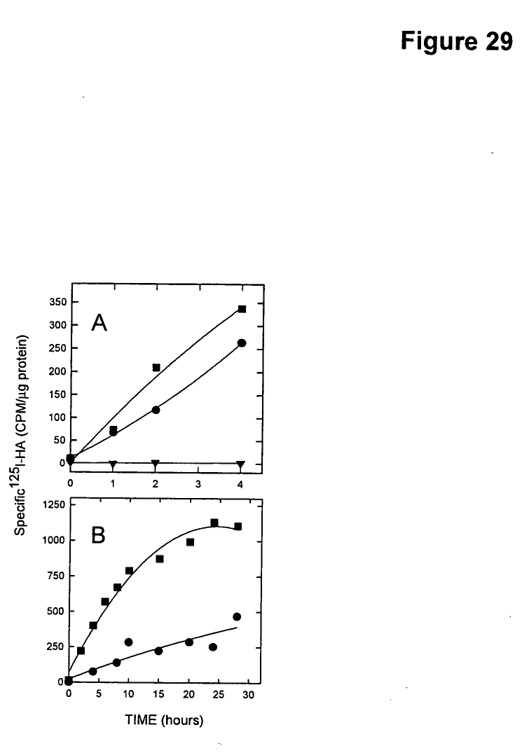

Consistent with the conclusion that the recombinant 190 kDa hHARE is a recycling receptor able to mediate the continuous endocytosis of ligand, the Flp-In 293 cell lines expressing hHARE, but not the vector-alone control, were able to internalize ¹²⁵I-HA for >20 h before cellular accumulation appeared to level off (FIG. 29). The apparent saturation of HA uptake is a steady-state situation, however, since cells are still endocytosing ¹²⁵I-HA, but they are releasing radioactive degradation products into the medium at the same rate. The ability of cells to process (i.e. internalize, degrade and secrete degradation products) large amounts of ligand over many hours or days is characteristic of recycling receptors that operate via the coated pit pathway (Weigel and Yik, 2002; Mellman, 1996; and Weigel, 1993).

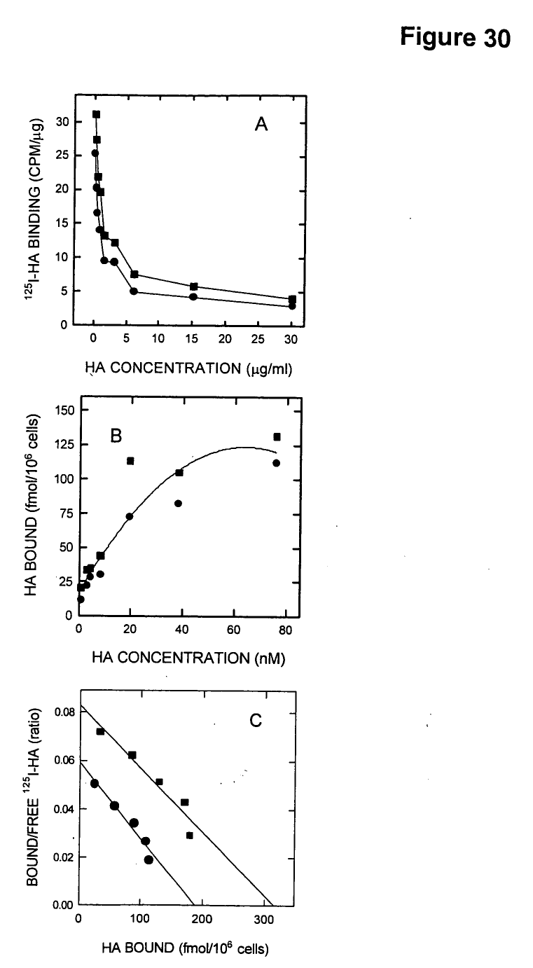

Since no ligand binding information exists for the individual hHARE species, equilibrium binding studies were performed using 190hHARE Flp-In 293 clones #9 and #14 to determine total receptor content and the affinity of the HA-hHARE interaction (FIG. 30). Based on the concentration of unlabeled HA required for half-maximal competition of ¹²⁵I-HA binding, the apparent Kₘ for HA binding is 1-2 μg/ml or about 10 nM (FIG. 30A). When these data were normalized for the specific radioactivity of the bound ¹²⁵I-HA at each point, the resulting binding isotherm was hyperbolic, which is typical of many receptor-ligand interactions, and binding approached saturation at >80 nM HA (FIG. 30B). When analyzed according to the method of Scatchard (Scatchard, 1949), the data in replicate experiments were best fit by a single straight line (cc≧0.9), indicating that a single class of noninteracting HA binding sites was present in digitonin-permeabilized cells (FIG. 30C). Based on two independent experiments with both clones #9 and #14 (n=8) the mean (±SD) Bₘₐₓ and K_{d}

TABLE IV
Surface and intracellular HA binding, and receptor recycling during endocytosis.

| | | | | | | | HARE |
|---|---|---|---|---|---|---|---|
| Clone | Surface | Specificity | Total | Specificity | Endocytosis | Specificity | recycling |
| # | cpm/μg | % | cpm/μg | % | cpm/μg | % | Endo/surface |
| 9 | 12.4 ± 1.9 | 63.3 ± 10.0 | 54.2 ± 15.8 | 75.2 ± 9.5 | 300.8 ± 34.8 | 90.1 ± 2.0 | 24.8 ± 4.2 |
| 14 | 11.9 ± 3.0 | 50.7 ± 17.3 | 62.7 ± 21.7 | 71.3 ± 10.8 | 354.4 ± 26.8 | 90.0 ± 2.1 | 32.3 ± 8.1 |
| EV13 | 1.3 | | 12.3 | | 1.6 | | 1.3 |
| Stable Flp-ln 293 cell lines transfected with empty vector (clone #EV13) or the 190 hHARE cDNA (clone #9 and #14) were grown to confluence, chilled on ice, washed with HBSS and incubated with medium containing 1.5 μg/ml ¹²⁵I-HA with or without digitonin as described in | | | | | | | |
| # Methods to assess total or cell surface binding, respectively. A set of parallel cell cultures was incubated at 37° C. for 4 h in medium containing 1.5 μg/ml ¹²⁵I-HA. Nonspecific binding or endocytosis was assessed in the presence of a 100-fold excess of unlabeled HA. | | | | | | | |
| # Specific values shown are the mean ± SE (n = 10 for clones #9 and #14) or the average of duplicates for clone #EV13. The receptor recycling ratio is the amount of specific HA endocytosis divided by the specific cell surface HA binding. Regardless of cell type, when | | | | | | | |
| # vertebrate cells are treated with digitonin under the conditions used here, ˜50% of the total cellular protein is lost, representing the cytoplasmic contents (Weigel et al., 1983). Therefore, the protein yield for permeable cells is about half that for intact cells. | | | | | | | |

values were 196±45 fmol of total HA binding sites/10⁶ cells and 7.2±1.2 nM, respectively. The Bₘₐₓ value corresponds to ˜118,000 total HA binding sites per cell.

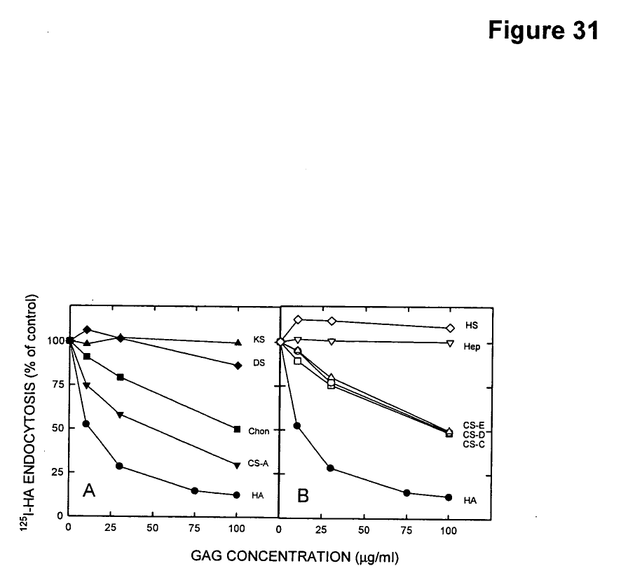

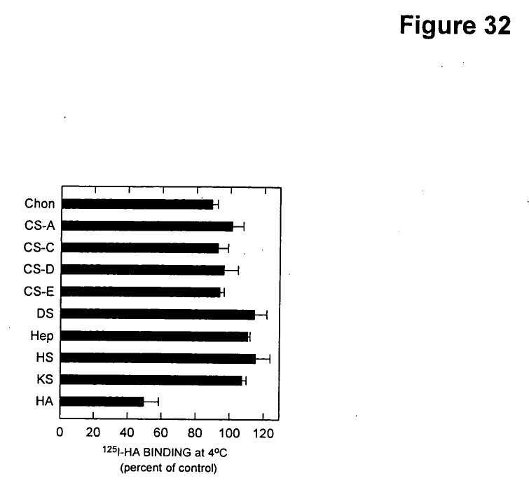

The GAG specificites of the two hHARE isoforms have not been determined. Using the stable 190 hHARE Flp-In 293 cell lines, the ability of individual purified GAG chains to block the endocytosis of ¹²⁵I-HA was examined. Even at 100 μg/ml, KS, HS and heparin did not compete for HA binding and uptake at 37° C., and DS showed a modest ˜15% inhibition (FIG. 31). This latter slight inhibition by DS appears to be significant, since it was observed in other experiments as noted below. CS-A was the most effective inhibitor, although its blocking ability was not comparable to that of HA, e.g. at 30 μg/ml ˜45% inhibition was observed with CS-A versus ˜70% inhibition with HA (FIG. 31A). Four other GAGs including chondroitin (FIG. 31A) and CS-C, CS-D, and CS-E (FIG. 31B) gave very similar titration profiles, with ˜50% inhibition at 100 μg/ml. For comparison, 100 μg/ml HA blocked ¹²⁵I-HA uptake by ˜87%. In contrast to these results at 37° C., none of the GAGs tested, except for HA, competed for ¹²⁵I-HA binding to 190hHARE Flp-In 293 cells at 4° C. (FIG. 32). ¹²⁵I-HA binding in the presence of the other nine GAGs ranged within 10% of the no-addition control value. The binding of GAGs other than HA to the 190 kDa hHARE protein, thus, appears to be very temperature dependent.

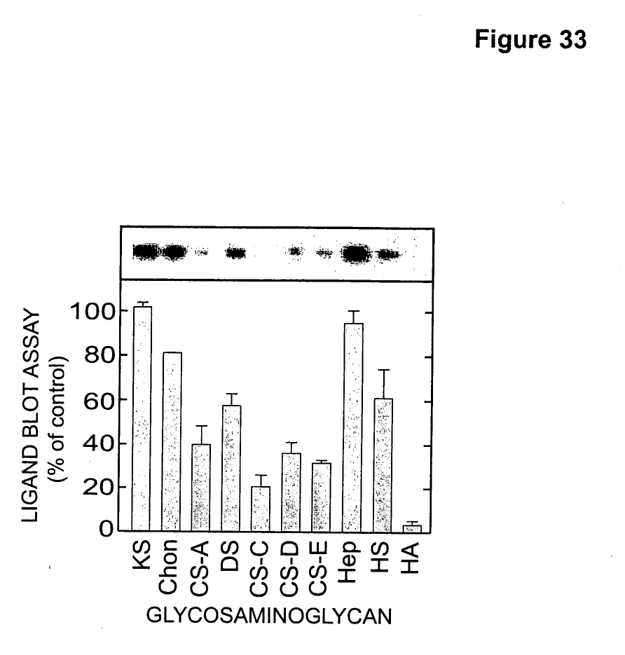

The ability of the 190 kDa hHARE to interact with GAGs was also assessed in a ligand blot format in which whole cell extracts were probed, in a Western blot format, with ¹²⁵I-HA (Yannariello-Brown et al., 1996). As shown in FIG. 27A, the level of hHARE protein expression in extracts is high enough to obtain an excellent signal, by autoradiography, in this assay within 6-18 hours. The ability of various GAGs to compete for ¹²⁵I-HA binding to the 190 kDa hHARE in the ligand blot assay (FIG. 33) closely paralleled the pattern seen for competition of endocytosis by cells, with the exception of HS. No competition was observed with KS or Hep. As with live cells, Chon, DS and all the CS types showed significant competition. In contrast, 100 μg/ml HS showed no effect on HA endocytosis in live cells (FIG. 31B), whereas 50 μg/ml HS inhibited ¹²⁵I-HA binding by 40% in the in vitro ligand blot assay.

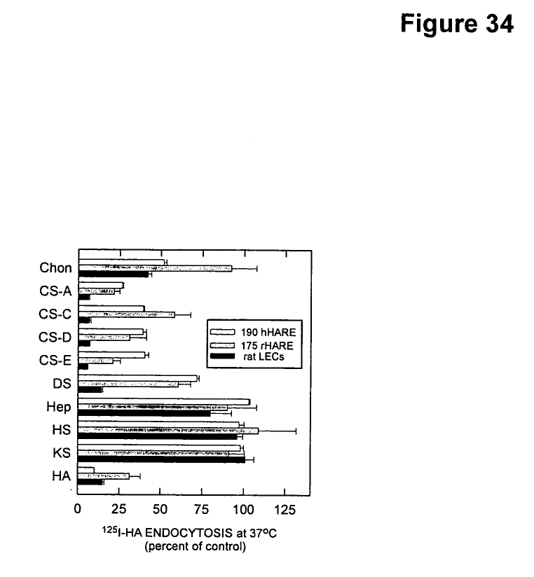

It should be informative to compare the GAG specificities of the rat and human small HARE isoforms because the amino acid sequences of the extracellular domains of these two proteins are 80% identical. Such GAG specificity differences might reflect significant differences between species in the biology of HARE or its role in GAG turnover. FIG. 34 compares the abilities of various GAGs to compete for ¹²⁵I-HA endocytosis by isolated rat LECs expressing both rat HARE isoforms, SK-HARE cells expressing the 175rHARE and Flp-In 293 cells expressing the 190hHARE. In each of the three cell types, little or no competition was observed with KS, HS or Hep. DS competed for HA uptake to the same slight extent (˜25-30%) in cells expressing either hHARE or rHARE. The four CS variants competed for HA uptake by both HARE proteins, although the patterns were not identical. The effects of CS-A and CS-D were essentially the same, whereas the preference for CS-C or CS-E was switched between the rat and human HARE proteins. The hHARE was competed better by CS-E, and the rHARE was competed better by CS-C. The greatest difference between the rat and human HARE was observed for competition by Chon. The hHARE appeared to interact more strongly with Chon (50% inhibition) than did the rHARE (˜10% inhibition).

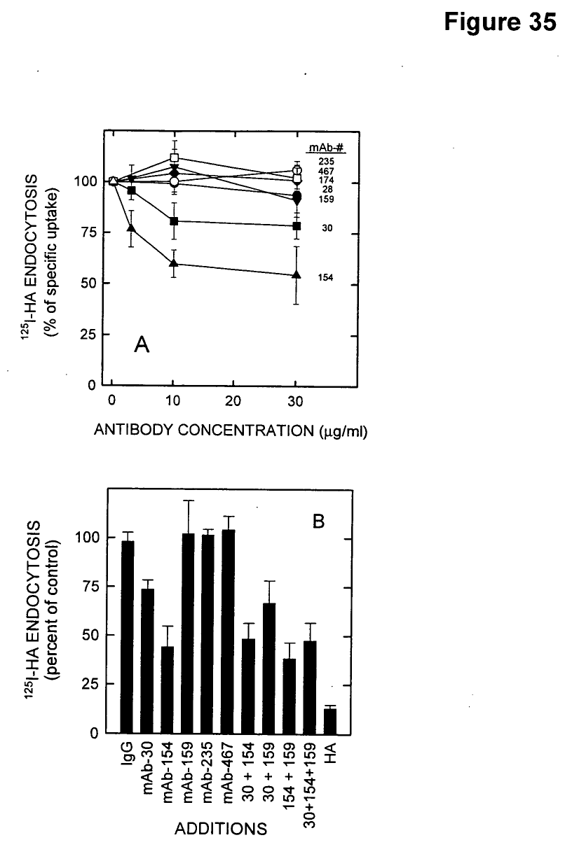

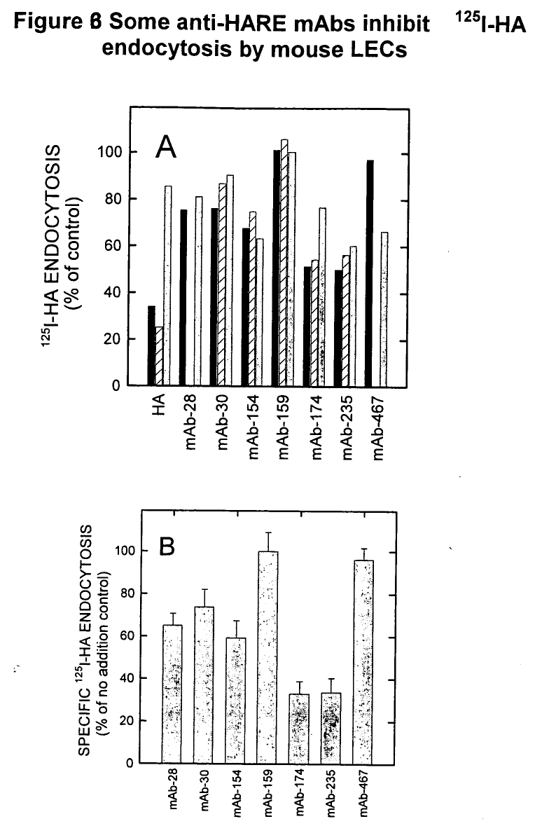

The development of a panel of eight mouse mAbs against the rat 175 kDa HARE protein to facilitate HARE purification and characterization was described in parent applications U.S. Ser. Nos. 09/842,930 and 10/133,172. Seven of these mAbs recognize both nonreduced rHARE proteins and were useful for a variety of immuno-procedures. In particular, mAb-174 was extremely useful, since it completely blocks HA binding to the rHARE in LECs, SK-HARE cells or in the ligand blot assay. A second mAb, #235, partially inhibited HA binding to rHARE to a level of ˜50%, indicating that HA binding likely involves multiple protein regions (epitopes). mAb-174 was used to demonstrate that HARE is responsible for the ability of liver to remove circulating HA, since this mAb blocked essentially all ¹²⁵I-HA uptake in a perfused liver system. Although mAb-174 and mAb-235 did not recognize hHARE, three of the seven anti-HARE mAbs (#30, #154 and #159) cross reacted with both native hHARE isoforms and with the recombinant 190 kDa hHARE (FIG. 27B). Nonetheless, the ability of any of the anti-HARE mAbs to inhibit the endocytosis of ¹²⁵I-HA by 190hHARE Flp-In 293 cell lines was tested (FIG. 35). Surprisingly, although mAb-159 had no effect on HA uptake (even at 30 μg/ml), partial inhibition of specific HA endocytosis was observed with both mAb-30 and mAb-154 (FIG. 35A). Negative controls for these effects included the other four anti-HARE mAbs (#28, #174, #235 and #467), as well as IgG and mouse serum (not shown), any of which caused ≦8% inhibition at concentrations up to 30 μg/ml. Experiments to assess the effects of various mAb combinations on HA uptake at 37° C. showed that the inhibitory mAbs (#30 and #154) were not additive (FIG. 35B). The maximum partial inhibition of specific HA endocytosis by mAb-30 or mAb-154 was, respectively, approximately 20-30% and 50-60%.

### Description of FIGS. 36-43

The results shown in FIG. 36 demonstrate that the function of HARE protein expressed by primary mouse sinusoidal liver endothelial cells is inhibited by the presence of anti-rat HARE mAbs 174 and 235. Although this inhibition is not complete, it is quite substantial at >60% inhibition of specific HA endocytosis for either one of these mAbs alone (FIG. 36B). In contrast, mAb-467 and mAb-159 showed virtually no inhibition. Several other mAbs (i.e. mAb-28, mAb-30, and mAb-154) gave intermediate levels of inhibition, blocking HA uptake in the range of 25% to 40%. The results demonstrate the ability of these mAbs, and in particular mAb-174 and mAb-235, to block the ability of mouse HARE to bind and endocytose HA. Thus, similar abilities of various anti-HARE mAbs to block HA binding to rat HARE (FIGS. 5, 6, and 19-21), human HARE (FIG. 35), and to mouse HARE (FIG. 36) have been shown; all of these results support the presently claimed and disclosed invention wherein Abs or mimetics are utilized to target and block the interaction between HARE and GAGs such as HA that it binds.

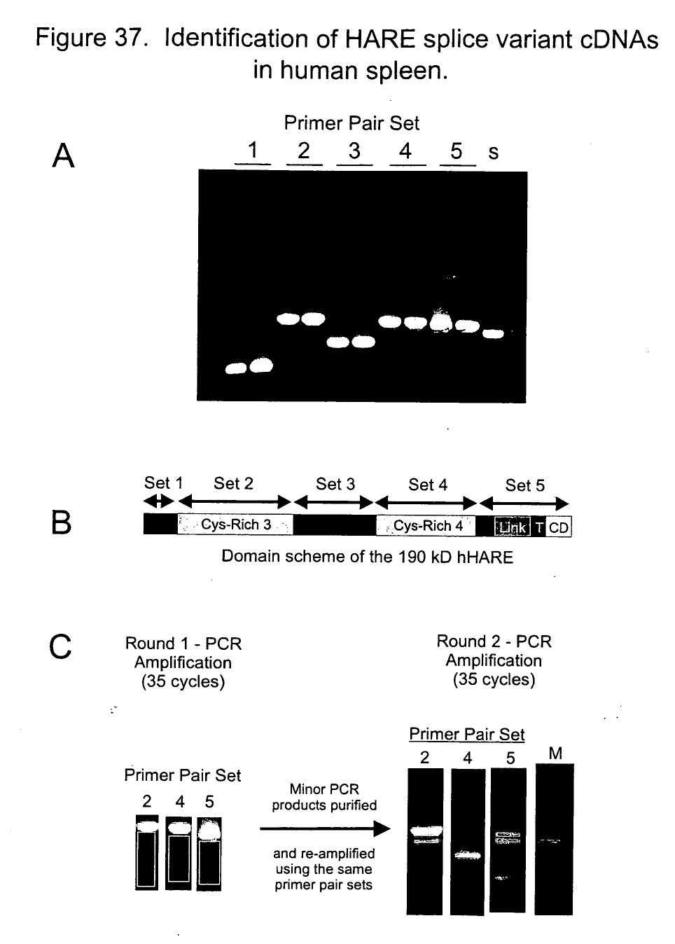

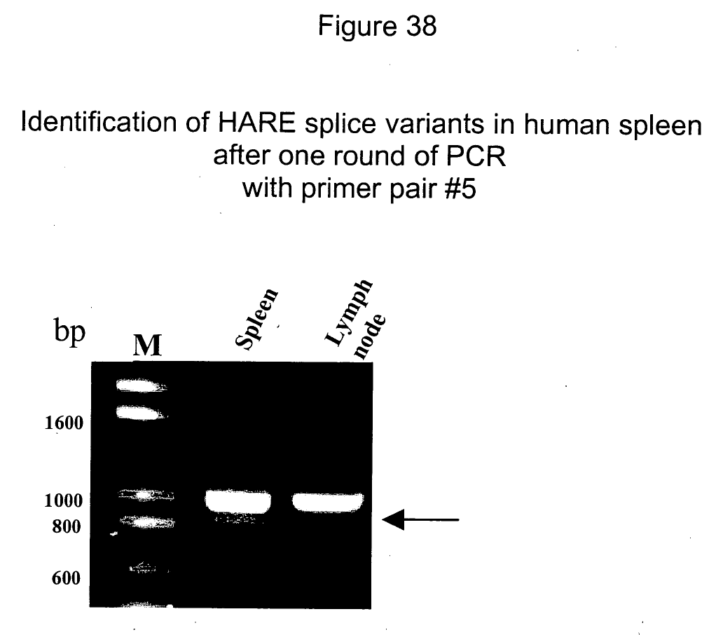

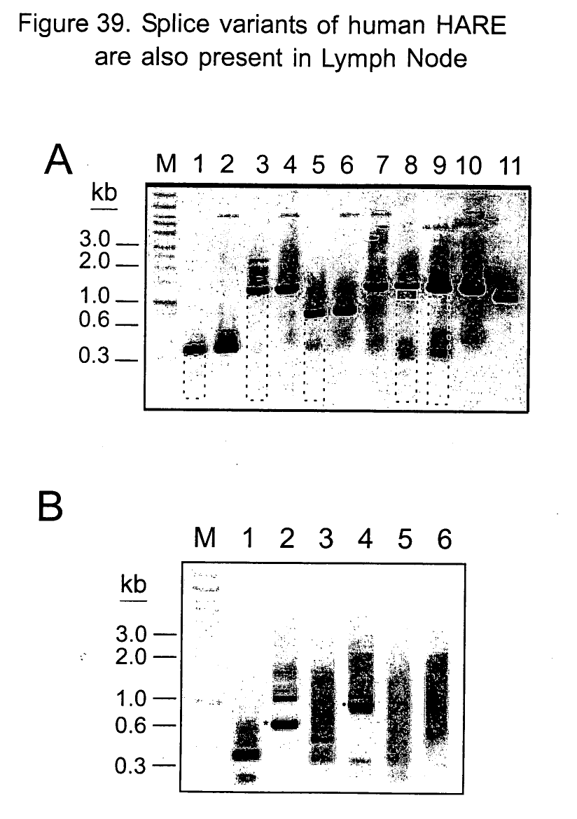

In initial experiments to identify hHARE splice variants, six candidate splice variants were found in spleen (FIGS. 37 and 38), and three different splice variants were found in lymph nodes (FIG. 39). Marathon cDNA pools (Clontech, BD Biosciences) were used as the templates for PCR reactions, as outlined in FIG. 37. Focusing on the region that encodes the 190 kD HARE protein, 5 sets of primers were used to amplify regions of <1100 bp that were present in the spleen cDNA pool. The top gel (FIG. 37A) shows the results of PCR amplification with the major product identified as the native wild type (wt) product. Analysis of the cDNA pool with each primer set (arrows) is accompanied by a positive control PCR reaction using the wild type 190-hHARE cDNA in an expression vector to indicate the migration position of the full-length product. In this first round of PCR (35 cycles), only the 5th reaction tube demonstrated a detectable minor fragment, which was not seen in lymph node (FIG. 38). The minor band and the gel regions beneath each major band in the experimental lanes in FIG. 37 were excised from the gel (FIG. 37C; white boxes on bottom left), purified, and subjected to a second round of PCR under the exact same conditions.

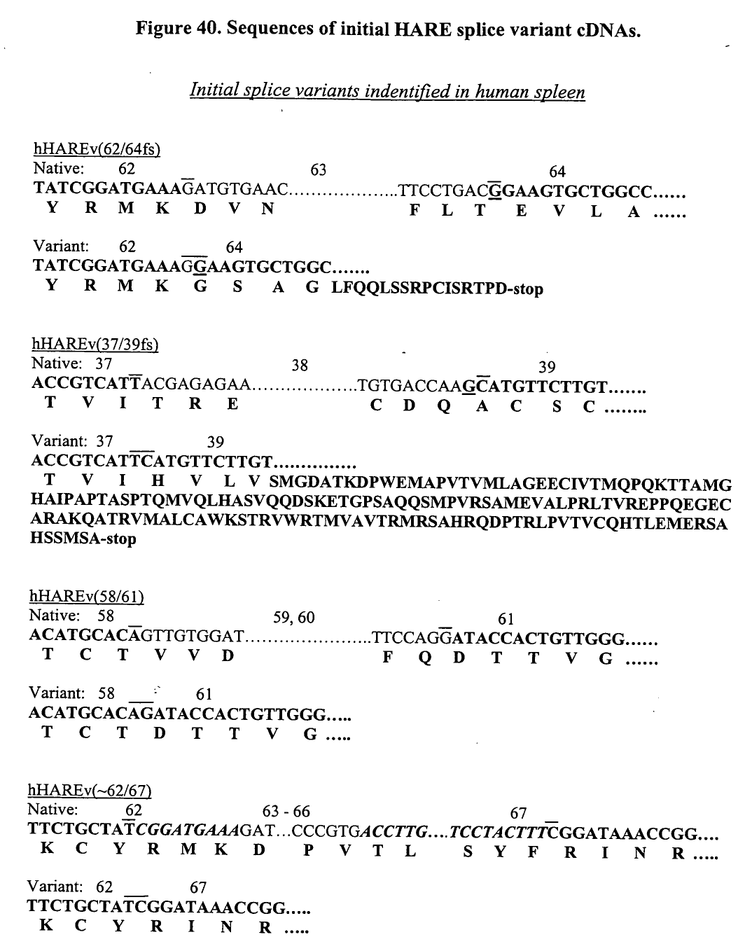

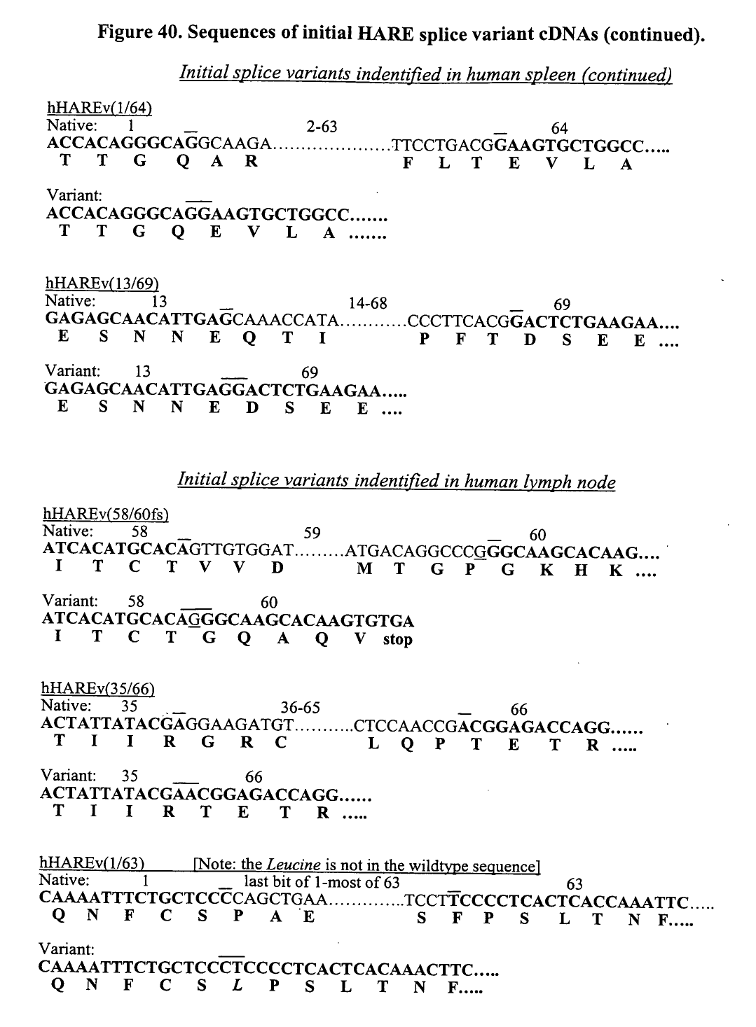

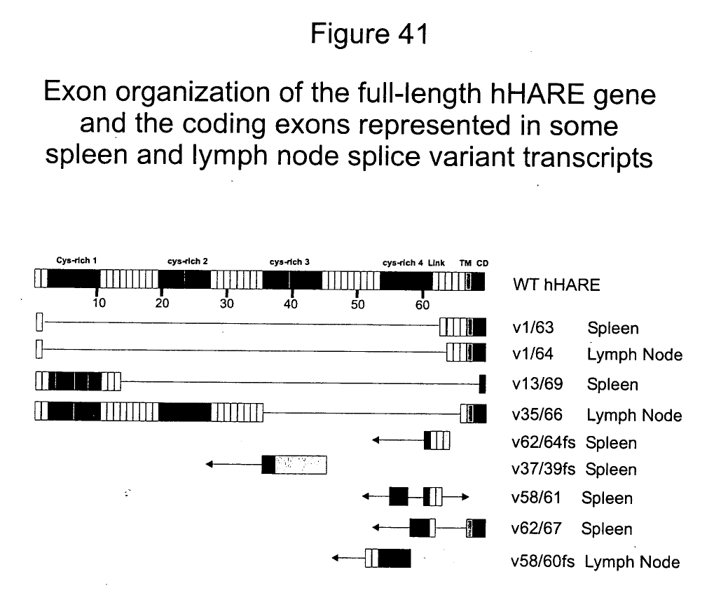

After separating DNA in a 1.0% agarose gel and staining with ethidium bromide, three of the five primer pair sets (FIG. 37C) yielded minor bands, some of which were sequenced and shown to be splice variants. FIG. 40 contains the sequences of the initial HARE splice variant cDNAs identified in human spleen and lymph node, and compares such sequences to the native HARE sequence. FIG. 41 shows a schematic of the different splice variants discovered thus far, and Table V gives more details for these nine splice variants. Some variants were evident even after one PCR round (as in FIG. 38).

Of the nine splice variants identified, the complete coding sequences have been determined for four of the splice variants. The full-length nucleic acid coding sequence and amino acid sequence of hHAREv(1/64) have been assigned SEQ ID NOS:55 and 56, respectively. The full-length nucleic acid coding sequence and amino acid sequence of hHAREv(13/69) have been assigned SEQ ID NOS:57 and 58, respectively. The full-length nucleic acid coding sequence and amino acid sequence of hHAREv(35/66) have been assigned SEQ ID NOS:59 and 60, respectively. The full-length nucleic acid coding sequence and amino acid sequence of hHAREv(1163) have been assigned SEQ ID NOS:61 and 62, respectively.

For the remaining five splice variants, the sequences of the splice regions have been determined, but a full-length fragment of each splice variant has not yet been cloned. The known splice region nucleic acid and amino acid sequences for hHAREv(62/67) are SEQ ID NOS: 63 and 64, respectively. The known splice region nucleic acid and amino acid sequences for hHAREv(58/61) are SEQ ID NOS: 65 and 66, respectively. The known splice region nucleic acid and amino acid sequences for hHAREv(37/39fs) are SEQ ID NOS: 67 and 68, respectively. The known splice region nucleic acid and amino acid sequences for hHAREv(62/64fs) are SEQ ID NOS: 69 and 70, respectively. The known splice region nucleic acid and amino acid sequences for hHAREv(58/60fs) are SEQ ID NOS: 71 and 72, respectively.

TABLE V
Candidate HARE splice variants identified from human spleen/lymph node cDNA pools

| | Exon(s) removed | Membrane- | Inclusion of full | Resident | Round of |
|---|---|---|---|---|---|
| Name of Variant | by splicing | bound or soluble | reading ORF | Organ | PCR detected |
| hHAREv(62/64)fs‡ | 63 | Soluble | No | Spleen | 1 |
| hHAREv(37/39)fs | 38 | Soluble | No | Spleen | 2 |
| hHAREv(58/61) | 59, 60 | Membrane | No | Spleen | 2 |
| hHAREv(˜62/67)* | 63-66 | Membrane | No | Spleen | 2 |
| hHAREv(1/63)* | 2-62 | Membrane | Yes | Spleen | 1 |
| hHAREv(13/69) | 14-68 | Soluble | Yes | Spleen | 1 |
| hHAREv(35/66) | 36-65 | Membrane | Yes | Lymph Node | 1 |
| hHAREv(58/60)fs | 59 | Soluble | No | Lymph Node | 2 |
| hHAREv(1/64) | 2-63 | Membrane | Yes | Lymph Node | 1 |

*indicates a splice variant that does not follow the standard splicing rules;
‡fs = frameshift

TABLE VI
Glycosaminoglycans (GAGs) used and their abbreviations

| | |
|---|---|
| Chon | chondroitin |
| CS | chondroitin sulfate |
| CS-A | chondroitin-4 sulfate |
| CS-C | chondroitin-6 sulfate |
| CS-E | chondroitin-4,6 sulfate |
| CS-D | chondroitin-2′,4 sulfate |
| DS | dermatan sulfate (CS-B) |
| HA | hyaluronic acid (hyaluronan) |
| Hep | heparin |
| HS | heparan sulfate |
| KS | keratan sulfate |

However, it is fully within the abilities of a person having ordinary skill in the art, given the present disclosure and specifically SEQ ID NOS:63-72, to clone and sequence full-length fragments of these five splice variants, and therefore the full-length sequences of the five splice variants also fall within the scope of the present invention. Based on the wild-type sequence, putative full-length coding sequences and amino acid sequences for these five splice variants have been constructed. The nucleic acid and amino acid sequences for putative full-length hHAREv(62/67) have been assigned SEQ ID NOS:73 and 74, respectively. The nucleic acid and amino acid sequences for putative full-length hHAREv(58/61) have been assigned SEQ ID NOS:75 and 76, respectively. The nucleic acid and amino acid sequences for putative full-length hHAREv(37/39fs) have been assigned SEQ ID NOS:77 and 78, respectively. The nucleic acid and amino acid sequences for putative full-length hHAREv(62/64fs) have been assigned SEQ ID NOS:79 and 80, respectively. The nucleic acid and amino acid sequences for putative full-length hHAREv(58/60fs) have been assigned SEQ ID NOS:81 and 82, respectively. However, it is to be understood that one or more changes to SEQ ID NOS:73-82 may be present in the actual full-length sequences of these five splice variants, and such changes are clearly identifiable to a person having ordinary skill in the art using the processes of cloning and sequencing a full-length fragment of a splice variant as described herein, and therefore changes to SEQ ID NOS:73-82 identified upon cloning and sequencing the full-length splice variants also fall within the scope of the present invention.

There are likely to be many more hHARE splice variants in spleen, lymph node and other tissues not yet examined, because many more candidate product bands that have not yet been sequenced and identified were observed (FIG. 37). In addition, different variants of the same size (amplified by the same primer pair) could be present in a given “product” band; these variants can be identified by isolating and sequencing individual PCR clones.

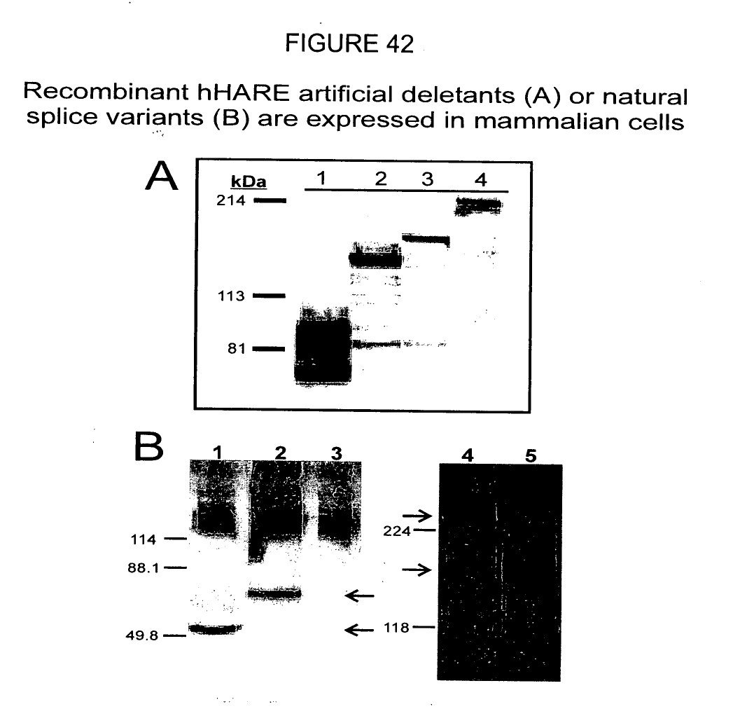

The constructs encoding designed variants hHARE(Δ1-89) (SEQ ID NOS: 83 and 84 for the nucleic acid and amino acid sequences, respectively), hHARE(A1485) (SEQ ID NOS:85 and 86 for the nucleic acid and amino acid sequences, respectively), hHARE(Δ1-695) (SEQ ID NOS:87 and 88 for the nucleic acid and amino acid sequences, respectively), and hHARE (Δ1-1063) (SEQ ID NOS:89 and 90 for the nucleic acid and amino acid sequences, respectively) have been made, sequenced and verified. The latter two constructs lack the C4 and C3/C4 domains, respectively. These truncated membrane hHARE proteins are all expressed in transiently transfected human Flp-In 293 cells (FIG. 42A), and recombinant proteins migrating at the expected size were detected by Western Blot analysis with the anti-V5 antibody (FIG. 42).

The results in FIG. 42 demonstrate that human cells are capable of expressing a wide range of natural splice or designed variants of hHARE. Although there will likely be exceptions in the future, all of the naturally occurring splice variants of the present invention tested so far have been expressed in cells transfected with appropriate vectors encoding their cDNAs. Four designed deletion variants lacking various portions of the amino terminal domains of hHARE were expressed by 293 cells, as indicated by the presence of protein bands of the expected size that contained the C-terminal recombinant V5 fusion epitope (FIG. 42A). Similarly, three splice variants, for which the complete predicted coding region has been determined, were also expressed from an appropriate expression vector (e.g. containing a membrane insertion signal), and in each case produced a protein of the expected size, that contained the recombinant V5 epitope. The finding that a wide range of hHARE protein variants can be expressed successfully, indicates that the protein likely contains many semi-independent domains along its length so that deletion of one or more of these domains does not dramatically hinder the ability of the remaining polypeptide to fold and establish disulfide bonds in a correct manner, to produce a native-like though truncated hHARE protein.

Cellular function of membrane-bound hHARE splice variants. Many of the variants identified so far (Table V) are predicted to be membrane proteins, whose cytoplasmic domains are targeted to coated pits (Weigel and Yik, 2002; and Mellman, 1996). These variant membrane receptors, which lack various regions of the wildtype hHARE protein sequence, are expected to bind and internalize a different subset of the various GAGs (Table VI) used to characterize the GAG-binding functions of the recombinant 175 kD rHARE (Zhou et al., 2002) and 190 kD hHARE in stable cell lines (FIG. 31). For example, of the 10 GAGs tested, all of the CS species, especially CS-A, are able to compete with the labeled HA, indicating their ability to bind to the 190 kD hHARE (FIG. 31A). The small hHARE isoform, therefore, has binding sites for multiple GAGs, and these sites overlap with the HA binding site(s). Similar functional studies were performed using ¹²⁵I-HA and cells transiently expressing the 190 kD hHARE (FIG. 26).

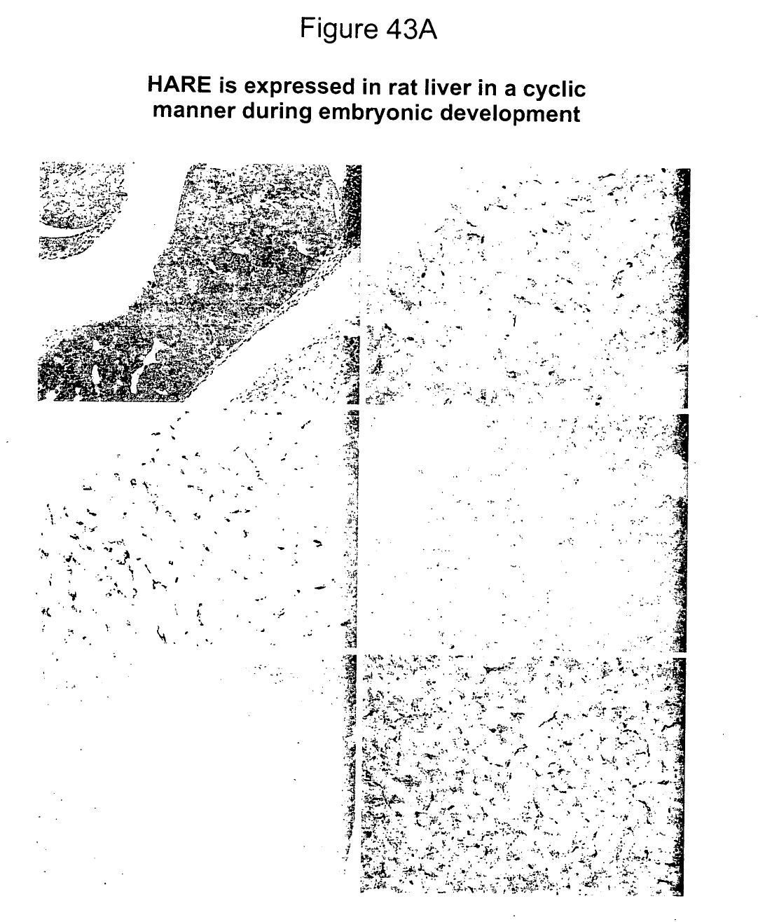

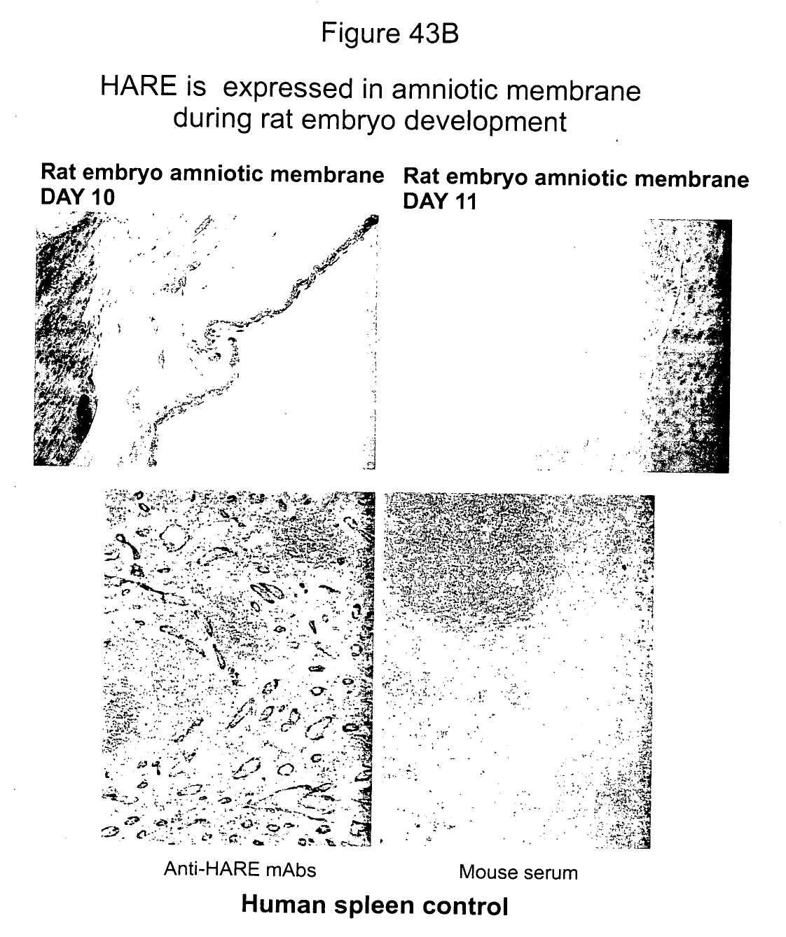

HARE is also expressed in bone marrow and in fetal liver. HA facilitates morphogenesis of the heart, skeleton, teeth, skin, hair, and other vertebrate organs. As previously disclosed, HARE is also present in human and rat bone marrow, and in early and late rat fetal liver (FIGS. 43A and B). As in liver, lymph node and spleen, HARE in marrow is localized to the sinusoidal endothelial cells lining the sinuses of these tissues. HARE expression during embryonic rat development is dramatically cyclic. Since HA plays predominant roles in embryonic development in vertebrates (Abatangelo and Weigel, (2000)), it was believed that HARE might be expressed in developing tissues that need to turnover and remove large amounts of HA or CS quickly. Thus HARE might be expressed in liver, perhaps reflecting a systemic turnover mediated by transfer of HA/CS through the circulation. HARE expression could also occur transiently in some fetal tissues (in order to mediate rapid local clearance of HA or CS or some other important function) even though expression in the adult tissue might be minimal or absent. Therefore, a set of slides prepared from day 10-18 rat embryos (Novagen) were obtained, and the presence of HARE was assessed by immunohistochemistry using a mixture of the eight anti-rat HARE mAbs described herein (FIG. 43A). HARE expression in fetal liver was up-regulated, down-regulated and then up-regulated again as development progressed from day-13 to day-18. HARE expression is evident at day-13 and very high on day-15 (FIG. 43A; left panel, middle row), becomes very low then absent on day-17 (FIG. 43A; left panel, bottom row), but then is very high again on day-18 (FIG. 43A; right panel, bottom row). HARE expression probably remains elevated from day-18 until birth at approximately day-21. Since gestation is at approximately day-20, the HARE expression at day-18 is likely the same as in adult liver. The significance of the oscillating expression of HARE is unknown, but it is believed that the earlier (day-15) expression is a fetal HARE variant and the later HARE expression (day-18) is the adult HARE form. Since fetal liver is the initial site of hematopoesis and HARE is also expressed in rat and human (FIGS. 12 and 13) bone marrow, it is possible that these tissues express specific hHARE splice variants with novel functions other than GAG clearance.

Although other proteins may undergo similarly large expression changes in such a cyclic manner, this fetal liver expression pattern is very striking. The only other tissue to demonstrate staining was day-10 amniotic membrane (FIG. 43B). Using the anti-HARE mAbs described herein, specific anti-HARE staining was also found in day-12 chick embryo liver (no other stages were examined). No staining was seen with mouse IgG. Therefore, HARE (or a related protein) is expressed in both rat and chicken during development.

Fetal liver and adult spleen and bone marrow are not the major sites of HA clearance mediated by HARE. This latter function is mediated by the small and large HARE isoforms expressed in liver and lymph node. HARE in these other tissues likely functions in alternate important processes, such as matrix organization, cell signaling, cell-cell and cell-matrix adhesion and/or hematopoesis.

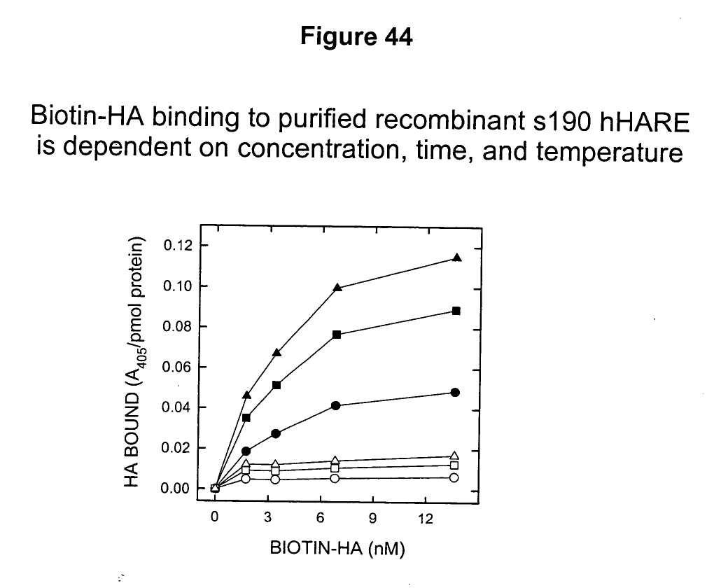

### Description of FIGS. 44-54

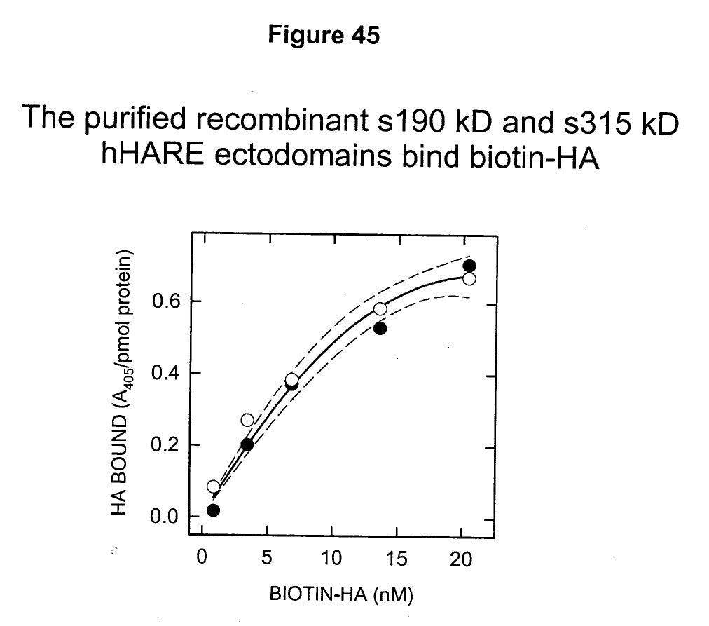

GAG-binding assays were conducted using soluble HARE domains. Assays using unlabelled GAGs and ¹²⁵I-HA to monitor binding, internalization or degradation (Zhou et al., 2002; Harris et al., 2004; and Weigel et al., 2003) are indirect competition assays. Many of the HARE splice variants will have differential binding specificities or affinities for the various GAGs. These variants will have complete or partial omissions of domains allowing for different ligand specificities. To test this, biotinylated GAGs were used in direct binding assays. Based on the weight-average mass of the 9 non-HA GAGs to be used (determined by light scattering; Harris et al., 2004), 1:1 molar ratios of GAG:biotin hydrazide were used to couple an average of ≦1 biotin per GAG chain. If sensitivity is low, this level of modification is increased as needed. Since the HA used (280 kD) is larger than the other GAGs (7-38.5 kD), the biotin-HA derivative was modified at a frequency of one biotin per 200 sugars. This biotin-HA was used to establish an ELISA-like assay using the purified recombinant extracellular domains of the 190 kD (FIG. 44) as well as the 315 kD hHARE proteins (FIG. 45). The HARE proteins, containing a C-terminal 6×His fusion, were purified using Ni-chelate columns, followed by SDS-PAGE and electro-elution of the specific hHARE protein from excised gel. The s190 kD hHARE nucleic acid and amino acid sequences have been assigned SEQ ID NOS:91 and 92, respectively, while the 315 kD hHARE nucleic acid and amino acid sequences have been assigned SEQ ID NOS:93 and 94, respectively.

The purified hHARE proteins are >95% pure based on silver staining of the final protein preparations. Biotin-HA binding to the adsorbed 190 hHARE ectodomain is saturable, and dependent on time and the concentration of each species (FIG. 44). As expected based on the temperature sensitivity of the wildtype rat HARE protein in primary rat liver sinusoidal endothelial cells, ligand blot assays, and the recombinant rat and human HARE proteins in stable cell lines, the binding of HA by HARE is temperature dependent. Little or no binding is observed at 4° C., whereas robust binding occurs at the normal cellular temperature of 37° C. (FIG. 44). Although one might expect that the larger 315 kD protein would bind more HA than the smaller 190 kD isoform, surprisingly the s190 kD hHARE and full-length s315 kD hHARE bound biotin-HA with the same apparent affinity and to the same extent (FIG. 45). This may be due to the relatively large size of the HA used (280 kD), which is almost as large as the 315 kD HARE protein; binding of one large HA may preclude binding of other HA molecules. This effect would decrease as the HA size decreased. Both hHARE isoforms would likely be able to bind multiple copies of considerably smaller HA (e.g. 30 kD).

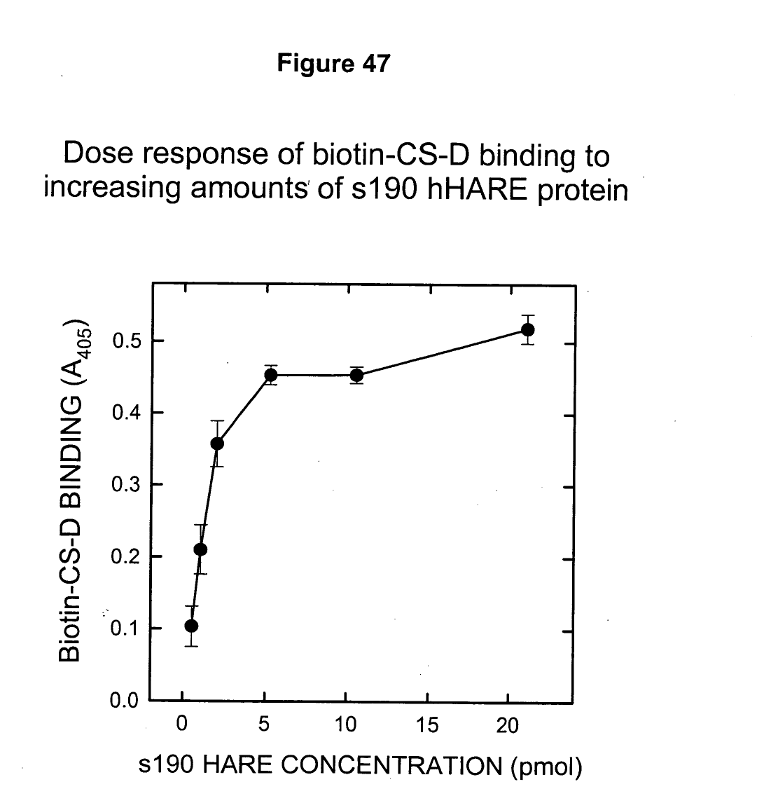

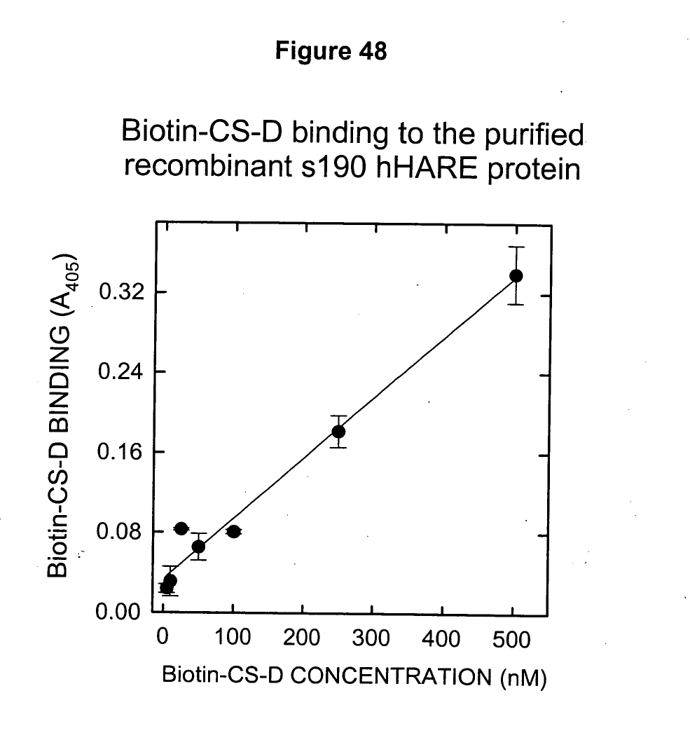

The results in FIGS. 44-51 clearly demonstrate the ability to characterize and quantitate the ability of any soluble hHARE variant to bind one or more of the various GAGs employed herein (Table VI). The ELISA-like assay uses purified HARE variants adsorbed to multi-well dishes, in which case the amount of test protein used can be varied, as shown in FIG. 47 in order to determine the dose response for binding of the desired biotin-labeled GAG. In this latter case, the amount of biotin CS-D binding displayed a very nice hyperbolic response as the amount of purified s190 on the surface of the test well was increased. The reproducibility of binding is greater, as expected, in the linear range of responsiveness, but then shows very little variability above the saturating level of adsorbed s190 protein (i.e. about 5 pmol). The linearity of HA binding to the s190 and s315 hHARE ectodomains (FIG. 44) also demonstrates the utility and responsiveness of this assay by showing the proportionality of biotin-HA binding as a function of increasing the amount of adsorbed hHARE protein. This linearity is also apparent in the binding of biotin-CS-D by purified recombinant s190 hHARE protein as shown in FIG. 48. In this case, the amount of biotin-CS-D binding was directly proportional to the concentration of biotin-CS-D tested over a broad range (i.e. up to 500 nM). Again, the reproducibility of replicates was very good. Thus, the above results show clearly that the interactions between one or more biotin-GAG species and a particular hHARE variant can be well established, monitored and quantified.

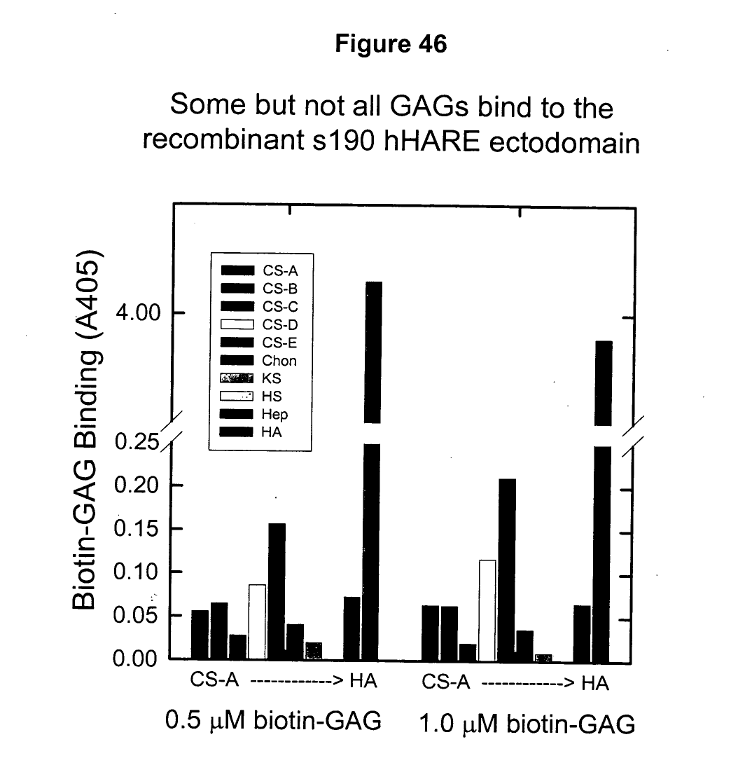

The direct GAG-binding results shown in FIG. 46 demonstrate that the purified s190 ectodomain of hHARE is able to bind to biotin-HA very well, but is also able to bind to other biotinylated GAGs, such as the CS types (e.g. CS-E and CS-D). The modest change in the amounts of bound GAG at the two different concentrations tested (i.e. 0.5 μM and 1.0 μM) indicate that these binding interactions may be at saturation with respect to the biotin-GAG concentration. These are the first experiments to measure directly (by use of multiple labeled biotin-GAGs) the ability of the 190 kD hHARE ectodomain to bind to a variety of individual GAGs. Thus, any of the desired GAGs can be biotinylated and used to characterize and quantify the binding ability of any desired hHARE variant. One of skill in the art will recognize the large number of possible GAG combinations that could be tested for binding to s190 hHARE in order to characterize the number and type of interactions that might occur between and among these species. It should be apparent that the hHARE variants, biotin-GAGs, and methods, assays and other procedures disclosed herein, can be employed to achieve whatever characterization is desired or necessary to practice the invention disclosed.

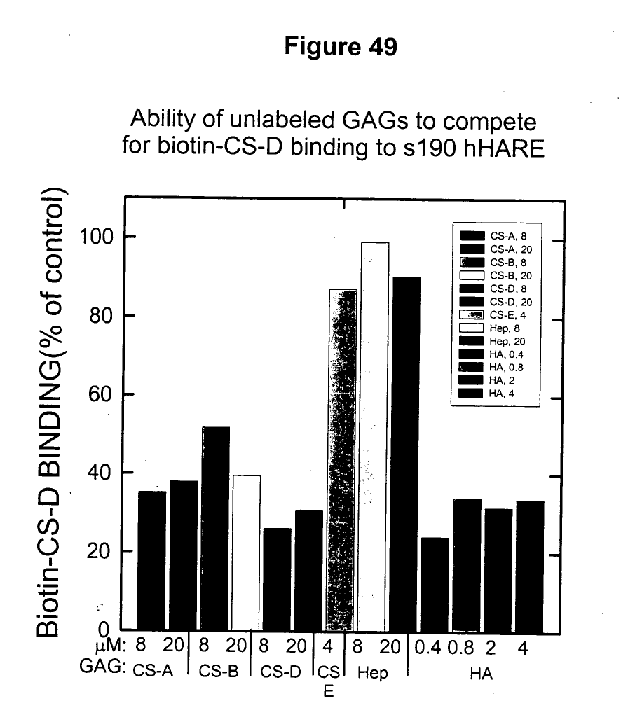

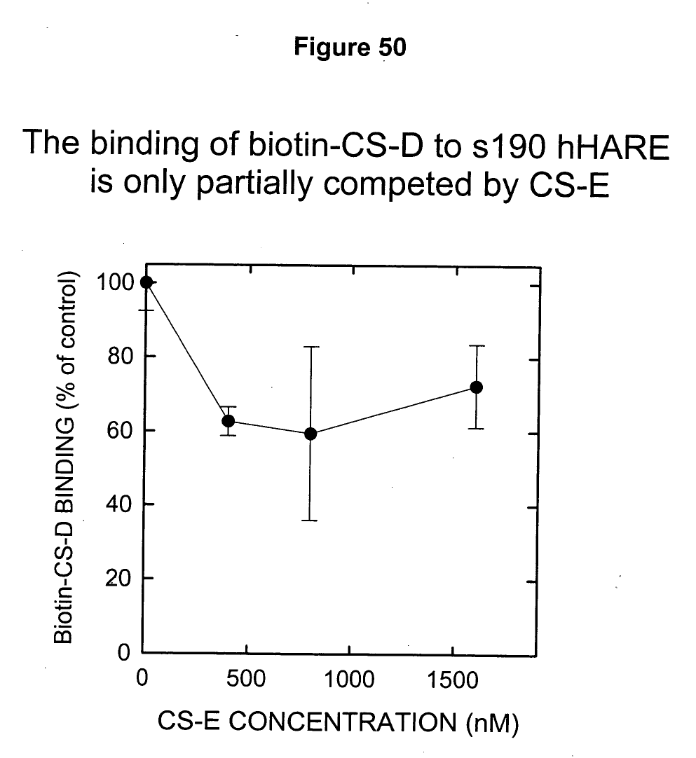

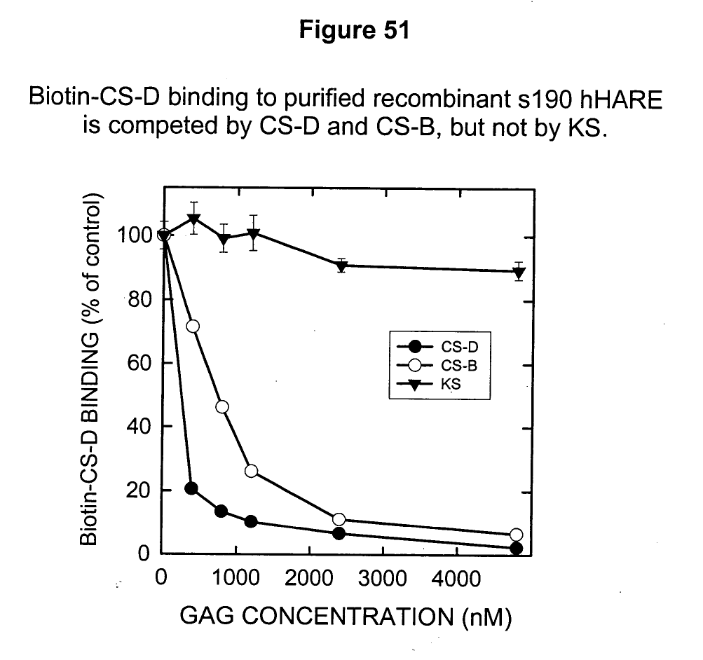

The 190 kD and 315 kD HARE proteins are organized into multiple domains along their length (FIG. 8), and these proteins are able to bind multiple types of GAGs. These wildtype native and recombinant proteins are thus not uniquely specific for one GAG, but are able to bind several GAGs as demonstrated in FIG. 31 for the endocytosis of HA by cellular 190 hHARE in the presence of other GAGs. Some particular domains of hHARE or variants thereof will be able to bind to only one type of GAG, and thus be uniquely specific for that GAG. In support of this, the results shown in FIGS. 49 and 50 indicate that the binding sites for CS-E and CS-D on hHARE are not completely overlapping. In indirect binding assays, one uses a competitor to displace the binding of a labeled GAG, such as biotin-HA. If a particular GAG, such as CS-A or CS-D in FIG. 49, is able to displace biotin-HA as completely as unlabeled HA, then some of the binding sites for both GAGs are most likely overlapping. In this case, then the binding of one GAG will adversely affect the binding of the second GAG. Such competition does not mean that hHARE does not have unique binding sites for the two GAGs, but that they overlap such that binding by one GAG precludes (e.g. for steric reasons) binding of the second GAG. Another possible reason for apparent competition is that a conformation change upon binding one GAG, e.g. CS-A, prevents the binding of HA. In this latter case, the binding sites for each GAG on HARE might be in separate domains. Therefore, if one GAG competes for the binding of a second GAG, one cannot know (without further experimentation and information) whether this mutual exclusion is due to overlapping binding sites or to conformational changes or perhaps to both effects. FIG. 51 provides an example of two GAGs (i.e. CS-D and CS-B) that compete completely for the binding of biotin-CS-D to the purified recombinant s190 hHARE protein. Based on the shape of the two competition curves, it appears that the apparent affinity of s190 hHARE for CS-B is slightly lower than for CS-D, since a higher concentration of the former was required to achieve an inhibition of 50% (which represents the approximate dissociation constant). In contrast, KS demonstrated virtually no inhibition of biotin CS-D binding (inverted triangles in FIG. 51), indicating that no KS binding sites overlap with those for CS-D.

However, when the result is that a second unlabeled GAG does not compete or competes only partially for the binding of a labeled GAG, then one can conclude for the former result that if the unlabeled GAG binds, it is to different binding sites, and for the latter result that both GAGs bind, but only partially to the same sites. The result shown in FIG. 50 clearly demonstrates that the ability of CS-E to compete for the binding of CS-D to purified recombinant s190 hHARE is only partial. At 400 nM CS-E, the inhibition of biotin-CS-D binding was ˜38% and despite increasing the CS-E concentration to 1.5 μM, the extent of inhibition did not change. Thus the ability of CS-E to compete for the CS-D binding sites within the s190 hHARE protein is only partial, and it is concluded that the two GAGs share some but not all of their binding sites on the protein. It will be apparent to one of ordinary skill in the art that the hHARE domains corresponding to CS-E binding only, to CS-D binding or to overlapping binding sites can be determined by a systematic investigation using the methods disclosed in the present invention. Thus, a hHARE variant capable of binding CS-E but not CS-D can be identified, expressed and purified for use in the present invention. Likewise, hHARE variants that bind the desired GAG or combination of GAGs can be identified, expressed and purified for use in the present invention.

One skilled in the art can now proceed to define the specific GAG binding domains within hHARE that recognize each of the seven GAGs able to bind HARE. HARE domains that recognize various combinations of HA, chon, CS-A, CS-B, CS-D, CS-E, and DS (CS-C) can thus be defined, created by means of recombinant DNA technology, expressed and utilized in a variety of ways, and therefore such HARE domains are fully within the scope of the present invention.

FIGS. 49 and 50 illustrate that CS-E does not effectively block CS-D binding to the recombinant s190 hHARE protein. The binding of biotin-CS-D to s190 hHARE (2.6 pmol per well) was assessed in the presence of no competitor (the 100% value) or the indicated amounts of unlabeled CS-A, CS-B, CS-D, CS-E, heparin or HA. All of the GAGs, with the exception of heparin and CS-E, competed effectively for the binding of CS-D, indicating that heparin and CS-E either do not bind to hHARE or do not bind to overlapping sites. Based on the results in FIGS. 46 and 50, it is clear that hHARE does not bind heparin (or binds very poorly), but does bind CS-E. The binding sites on hHARE for CS-D and CS-E, therefore, do not overlap completely indicating that both GAGs could bind to the same hHARE molecule.

FIGS. 49 and 50 illustrate that CS-E and CS-D do not bind to the same sites on the recombinant s190 hHARE protein. In FIG. 50, Biotin-CS-D (400 nM) was allowed to bind to adsorbed purified s190 hHARE protein (2.6 pmol per well) in the presence of no competitor (the 100% value) or increasing amounts of unlabeled CS-E as indicated. The partial ability of CS-E to block the binding of CS-D to s190 hHARE demonstrates that the two GAG binding sites are not completely overlapping, and both GAGs can be bound simultaneously.

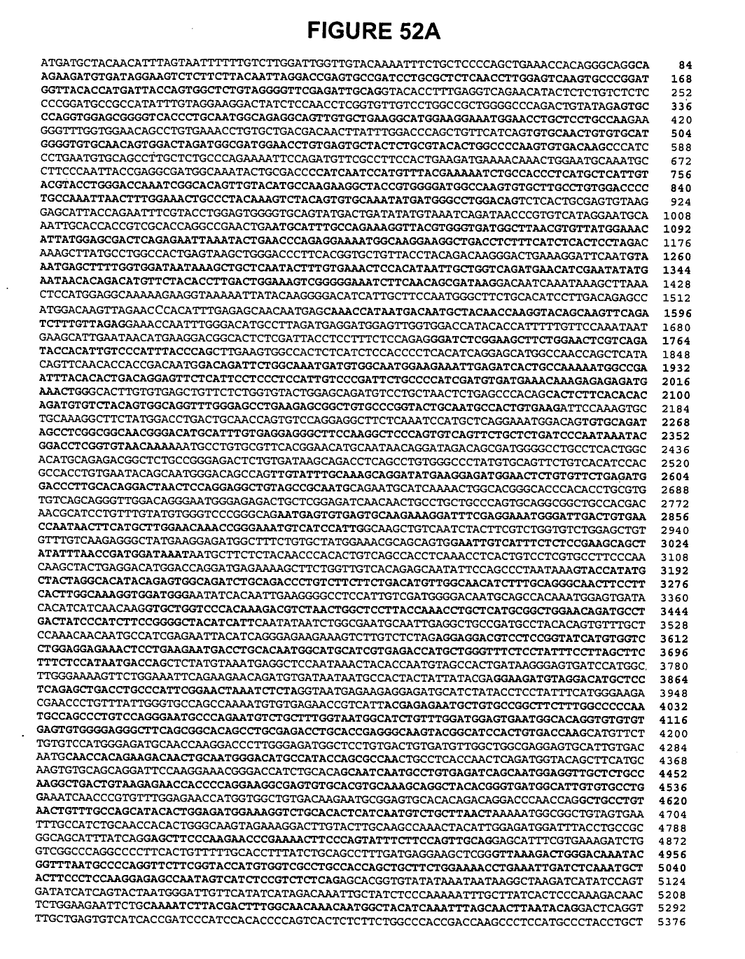

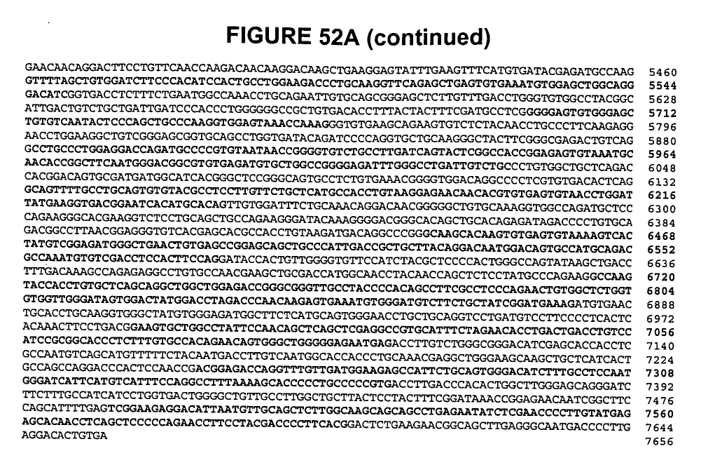

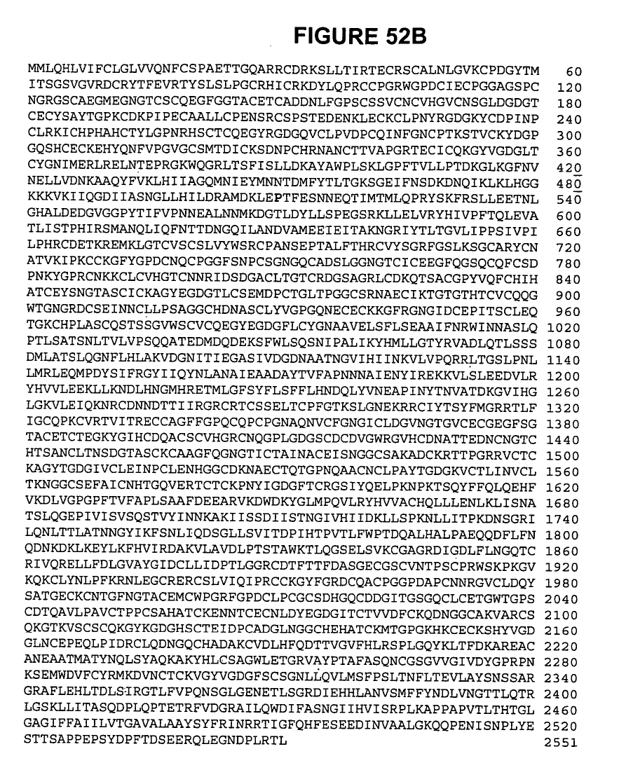

FIG. 51 illustrates that Biotin-CS-D binding to s190 hHARE is competed by CS-D and CS-B but not KS. Biotin-CS-D (400 nM) was allowed to bind to adsorbed purified s190 hHARE protein (2.6 pmol per well) in the presence of no competitor (the 100% value) or increasing amounts of either unlabeled KS, CS-B or CS-D as indicated. Unlike the results in FIG. 49 with CS-E, both CS-D and CS-B are able to completely block the binding of biotin-CS-D. The CS-B and CS-D binding sites on s190 hHARE appear to be largely overlapping. KS competes very poorly for binding of biotin-CS-D, indicating that this GAG is not bound by hHARE, a conclusion supported by the failure of KS to block HA binding and uptake by cells expressing wildtype 190 kD hHARE (FIG. 31A). FIG. 52 illustrates the nucleic acid coding sequence of the full-length human HARE/Stab2 cDNA (FIG. 52A, SEQ ID NO:95), as well as the amino acid sequence of the human HARE precursor protein (FIG. 52B, SEQ ID NO:96).

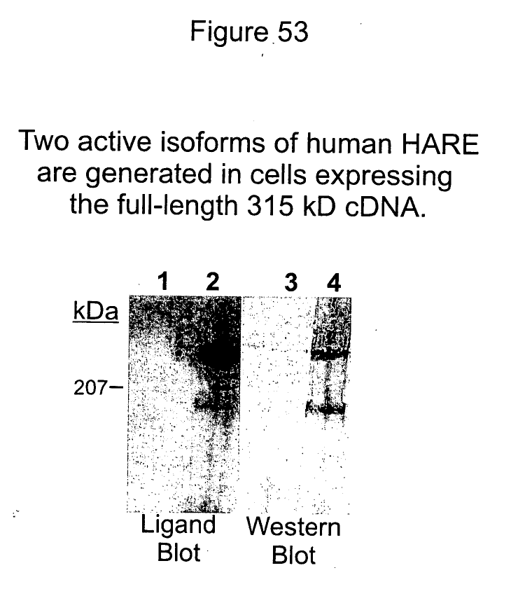

FIG. 53 illustrates that two active isoforms of human HARE are generated in cells expressing the full-length 315 kD hHARE cDNA (SEQ ID NO:95). Stable Flp-In 293 cell lines were isolated after transfection with a vector containing the full-length human HARE cDNA and selection with Hygromycin B. Detergent lysates from several stable cell clones expressing HARE were pooled (lanes 2 and 4), and the HARE proteins were immunoprecipiated using a mixture of three mAbs coupled to Sepharose 4B (mAbs 30, 154 and 159 which recognize hHARE). A ligand blot assay was performed using ¹²⁵I-HA followed by autoradiography (left panel). The same membrane was then subjected to Western Analysis (right panel) using rabbit anti-V5 antibody to detect the epitope tag on recombinant HARE proteins. The two HARE proteins apparent in lane 4 were both active, i.e. able to bind HA, and correspond to the previously identified native hHARE 190 kDa and 315 kDa isoforms. The results indicate that the smaller hHARE isoform is derived from a larger precursor produced from the full-length protein.

An artificial spleen/lymph node cDNA for the full-length 315 kDa hHARE was created in order to assess its functionality and GAG specificity in stable cell lines. The results in FIG. 53 demonstrate that transfected cells express the full-length hHARE protein as expected; the protein is at the correct size and is reactive with the anti V5 antibody, which is present at the C-terminal end of the recombinant protein as a fusion protein (FIG. 53, lanes 3 & 4). Furthermore, the recombinant 315 hHARE protein is folded correctly, since it is active and able to bind HA in the ligand blot assay (FIG. 53, lanes 1 & 2) and the protein migrates slower in SDS-PAGE after reduction, indicating the presence of disulfide bonds. In further support of the previous findings that the 190 kD hHARE is derived normally from the larger 315 kD protein or related precursor, it was found that all cell lines expressing the recombinant 315 kD hHARE protein also express a smaller form of the HARE protein that migrates at −190 kD, also contains the C-terminal V5 epitope, and is active as an HA-binding protein in the ligand blot assay (lanes 2 and 4 in FIG. 53). Thus, human 293 cells appear to have the necessary processing machinery to generate the smaller hHARE isoform from the larger 315 precursor protein.

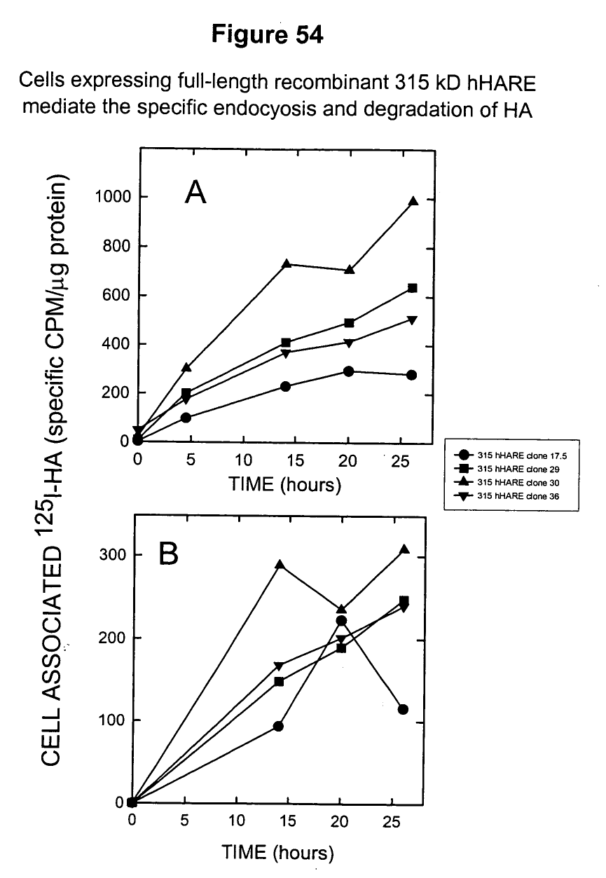

The results in FIG. 54 demonstrate that cell lines expressing the recombinant full-length 315 hHARE protein are capable of specifically endocytosing labeled HA and delivering it to lysosomes for degradation. These cell lines likely behave in a similar way to those expressing the 190 hHARE isoform, which were more extensively characterized in FIGS. 26-35. Previous analysis of 190 hHARE cell lines confirmed that cells transfected with vector alone are not capable of mediating HA uptake and degradation. The four 315 hHARE-expressing clones examined in FIG. 54A all demonstrated specific endocytosis of HA, although the rates of uptake varied among the cell lines. For example, HA uptake by clone #30 was about twice that for clone # 17.5. All four clones were also capable of degrading the internalized HA as indicated in FIG. 54B, and the trend was similar in that more degradation products were generated by clone #30 compared to clone #17.5. These characteristics are consistent with the ability of the larger 315 hHARE isoform to function as an endocytic, recycling receptor, which is able to mediate the uptake and degradation of a variety of GAGs, including HA.

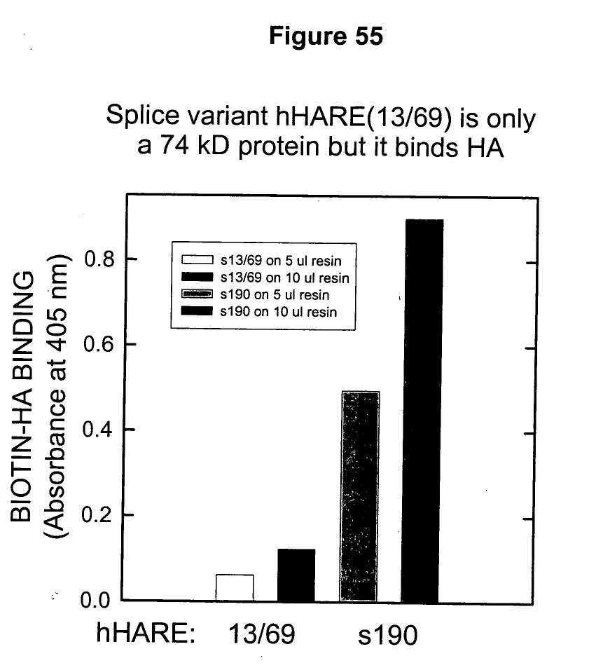

The result in FIG. 55 is particularly relevant to the invention disclosed herein, since it demonstrates clearly the ability of a small hHARE region to retain the ability to bind HA. Thus, although very high affinity HA binding may be greater for a full-length 190 kD or 315 kD hHARE, even a fragment that is only 74 kD shows significant HA binding. Since the conditions (e.g. pH, divalent cations, ionic strength, or concentration) for HA binding by hHAREv(13/69), a variant lacking the protein portions encoded by exons 14 through 68, have not been optimized, it is possible that the HA binding ability of this splice variant is even greater than shown here.

### Discussion

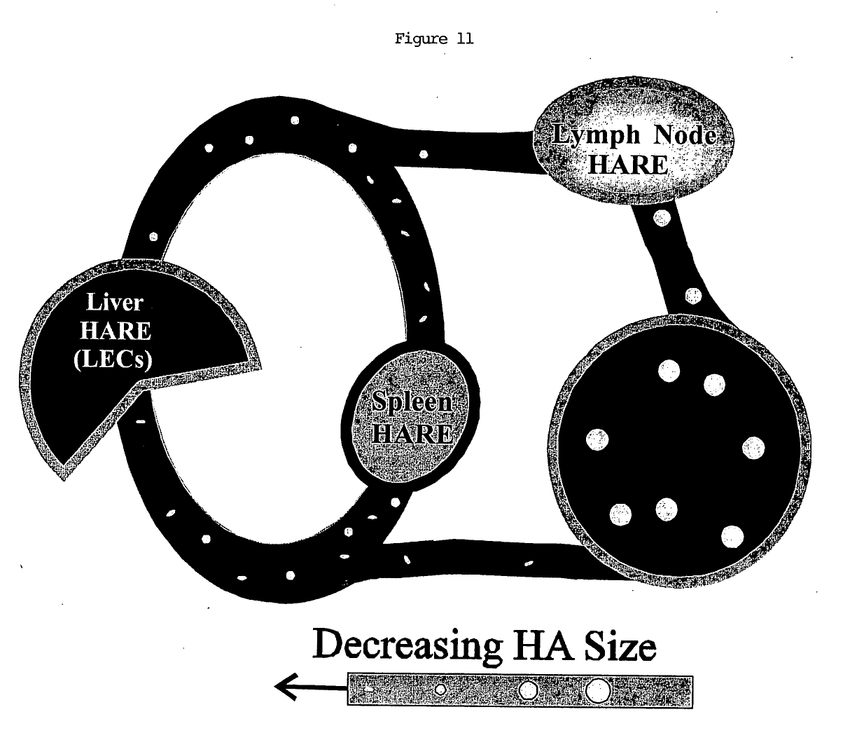

HA and CS turnover continuously in ECMs throughout the body (see FIG. 11). For humans, the HA turnover rate is so fast (e.g. ˜24 h in skin) that about one-third of total body HA is degraded and resynthesized daily (Laurent and Fraser, 1991). Partially digested native HA molecules are released from tissue matrices as large HA fragments of ˜106 Da that would still contain bound aggregating PGs (e.g. aggrecan or brevican) and Link proteins (Tzaicos et al., 1989; Lebel et al., 1988; and Laurent et al., 1991). The released ECM fragments would also contain covalently attached CS and other GAG chains, as well as a variety of bound ECM proteins and growth factors. Thus, multiple components associated with these HA-PG fragments are simultaneously released from an ECM and then enter lymphatic vessels and flow to regional lymph nodes. Lymph nodes are the initial and primary sites for the clearance of the HA and CS, accounting for ˜85% of the HA degradation. Liver is the second clearance site, after the lymph node effluent enters the circulation, accounting for ˜15% of the total body HA, and presumably CS, turnover. The clearance and degradation of HA and CS in liver and lymph nodes is mediated by HARE, which is expressed in the sinusoidal endothelial cells of these tissues (Zhou et al., 2000; Zhou et al., 2002; Zhou et al., 2003; Fraser et al., 1981; Fraser et al., 1983; and Eriksson et al., 1983).

Although no studies have yet addressed its role in normal health and in various diseases or pathologies, HARE is likely to be important in human physiology. Despite the high turnover rate of HA, the normal steady-state concentration of HA in blood (i.e. 10-100 ng/ml) is very low (Laurent and Fraser, 1992; and Laurent et al., 1991). The HA/CS clearance systems utilizing HARE in lymph node and liver, therefore, function very efficiently, indicating that the removal of HA from lymph fluid and blood is important for normal health. First, one would predict that if HA levels increased, particularly if the HA mass was large, then the increased viscosity of blood might create potentially adverse situations, e.g. erythrocyte passage in narrow microcapillaries could be impaired. Second, since HA binds to human fibrinogen (LeBoeuf et al., 1986) and stimulates fibrin clot formation in vitro (LeBoeuf et al., 1987), elevated HA levels could alter normal coagulation homeostasis. Finally, several diseases including some cancers (Thylen et al., 1999), psoriasis (Lundin et al., 1985), scleroderma (Freitas et al., 1996), rheumatoid arthritis (Manicourt et al., 1999), and liver cirrhosis (Yamada et al., 1998; and Lai et al., 1998) are associated with elevated levels of HA in serum. Over the last decade, numerous studies have suggested that the HA clearance function of liver can be used as a diagnostic tool to detect and monitor liver failure (Bramley et al., 1991). This hepatic function of LECs may also be a prognostic indicator of success in liver transplant patients.

In the disclosed and claimed present invention, artificial cDNAs were created in order to express a desired recombinant form of the large or small spleen hHARE isoform in stable cell lines. This enabled the characterization, for the first time, of the GAG specificity and endocytic activity of the small hHARE isoform in the absence of the larger hHARE isoform. Several key characteristics of the 190 kDa hHARE are very similar to those of the 175 kD rHARE. Both smaller HARE isoforms are functional endocytic HA receptors with the appropriate, as yet unidentified, sorting signals for targeting HARE to coated pits and then through an intracellular receptor recycling itinerary. Each HARE, though expressed in different cell types, mediated the continuous endocytosis of HA and its delivery to lysosomes for degradation. The rate of hHARE recycling in Flp-In 293 cell lines (i.e. one cell surface equivalent per 7-9 min) was comparable to that determined in primary rat LECs (McGary et al., 1989). The apparently slower rate of rHARE recycling in SK-Hep-1 cell lines (˜20 min) is likely due to a decreased capacity of the coated pit pathway in this latter cell line (Zhou et al., 2002; Yik et al., 2002; and Yik et al., 2002), rather than to intrinsic differences between the recombinant rHARE and hHARE. The affinities of the smaller rHARE and hHARE were also very similar at 4.1 nM and 7.2 nM, respectively.

Two significant differences between the rat and human HARE proteins that might be related are their slightly different GAG specificities and their very different profiles for anti-HARE mAb inhibition of HA-binding. The 190 kDa hHARE has a broad specificity for sulfated and nonsulfated GAGs, yet this recognition is not indiscriminant, because HA binding is not affected by KS, HS, or Hep. Thus, the 190 kDa hHARE recognizes HA and chondroitin, the two least negatively charged GAGs, as well as three CS variants with different levels and patterns of sulfation. The two GAGs with the greatest negative charge, HS and Hep, are not recognized. In contrast, all the CS variants tested were able to compete for HA binding. Though the pattern of inhibition by CS variants was similar to that for the 175 kDa rHARE, it was not identical. In particular, the two HARE species differ quantitatively in their recognition of CS-C and CS-E.

The GlcUA-GlcNAc disaccharide units in HA and the GlcUA-GalNAc disaccharide units of chondroitin were recognized by the 190 kDa hHARE. HARE also recognized all of the sulfated CS types tested, despite differences in the position and number of sulfates among their disaccharide units. Chondroitins sulfated at GalNAc positions C4, C6 or C4, 6 (i.e. CS-A, CS-C and CS-E) or at C6 of GalNAc and C2 of GlcUA (i.e. CS-D) were effective competitors of HA binding, although none were as effective as HA. CS-A was a slightly better competitor than the other CS types, all of which were essentially identical. DS (also called CS-B) was the weakest competitor. KS contains Gal rather than a uronic acid and was not a competitor. Although Hep and HS are very highly sulfated GAGs, they were not able to compete HA binding to the 190 kDa hHARE. Perhaps HARE can recognize N-acetyl groups in the amino sugars of some GAGs, but not the N-sulfated glucosamine residues characteristic of Hep and HS.

The greatest difference in GAG specificity between the rat and human proteins was with Chon, which poorly competes for HA binding by the rHARE. The 190 kDa hHARE protein appears to recognize Chon almost as well as most of the CS types. These slight differences between species in relative preference for various GAGs may be reflected in the more dramatic differences in the inhibition of their HA binding ability by mAbs. HA binding and endocytosis by the small (or large) rat HARE proteins is completely blocked by mAb-174 and partially blocked by mAb-235, whereas no inhibition of ligand binding or uptake was observed with the other five anti-HARE mAbs that recognize these proteins in Western blots and various immuno-procedures. In contrast, it has been demonstrated herein that mAb-30 and mAb-154 partially inhibit HA binding and endocytosis by the 190 kDa hHARE. The other anti-HARE mAbs, including mAb-174 and mAb-235, were not inhibitory. Thus, although mAb-30 and mAb-154 bind to both rHARE and hHARE, this binding only inhibits HA recognition by the latter protein.

Another distinctive and unusual feature of the 190 kDa hHARE was that GAG inhibition of its binding to HA was temperature sensitive. Although multiple GAGs were able to block the binding and endocytosis of ¹²⁵I-HA mediated by cells expressing recombinant hHARE at 37° C., none of these GAGs could compete for ¹²⁵I-HA at 4° C. Presumably, the extracellular domain of the 190 kDa hHARE undergoes a substantial conformational change between 37° C. and 4° C. that virtually eliminates the binding of GAGs other than HA. However, these binding studies were indirect and only monitored the binding of ¹²⁵I-HA, it is possible that a conformational change could also create a situation wherein both GAGs might bind to separate sites without, e.g. steric, interference. Distinguishing between these possibilities must await further direct binding studies between the various GAGs and the purified 190 kDa hHARE or its extracellular domain.

Taken together the above results indicate that amino acid sequence differences between the small rat and human HARE proteins may alter their GAG specificity slightly. These sequence differences may also alter conformations that occur when mAbs bind to the proteins, so that GAG binding is affected indirectly, and in a species dependent way, by formation of a mAb-HARE complex. It is likely that mAb-30 and mAb-154 recognize epitopes that are not directly involved in HA binding by hHARE, but that steric factors limit interactions of the protein with HA. The finding that inhibition with either mAb is only partial indicates that hHARE likely contains multiple HA-binding regions. It also indicates that each hHARE protein may bind more than one HA molecule. Again, these or other possible explanations will require epitope mapping studies and studies to define the GAG-binding regions within the extracellular domain of HARE.

The results in FIGS. 49 and 50 also support the idea that hHARE protein contains multiple independent GAG-binding domains, for example that recognize CS-D or CS-E, and that one of ordinary skill in the art will be able to identify such HARE protein variants or peptides that display a unique desired GAG specificity or a desired combination of GAG-binding activities. These hHARE variants with desired GAG binding properties for the purpose of one or more inventions disclosed herein can then be transiently or stably expressed by cells transfected with appropriate expression vectors containing coding cDNAs for the desired HARE variant in order to produce the HARE protein. A preferred embodiment is for production of soluble HARE variants with one or more desired binding activities for HA, chon or a CS type. Stable cell lines expressing the desired HARE variant protein can be grown on a large scale, e.g. in roller bottles, hollow fiber cell culture systems, or other similar device. The secreted HARE variant protein can then be purified from the collected cell culture medium by the use of established procedures know to those with ordinary skill in the art. For example, immunopurification using monoclonal or polyclonal antibodies against the HARE protein or against a fusion peptide or protein, typically fused at one end of the HARE protein. Other methods may also be useful for such purification including preparative HPLC, employing size exclusion, ion-exchange, hydrophobic or other interactive chromatography. Metal chelate chromatography can also be employed as described in the present invention to purify HARE variants containing a His-6 amino acid sequence added as a fusion protein.

### Methods

General. Protein content was determined by the method of Bradford (1976) using BSA as a standard. SDS-PAGE was performed according to the method of Laemmli (1970). Western blotting was performed as described by Burnette (1981) with minor modifications (Zhou et al., 2000). DNA sequencing was performed by the dideoxy nucleotide method (Sanger et al., 1977) either manually using the thermo sequenase radiolabeled terminator cycle sequencing kit or by the Department of Microbiology & Immunology Sequence Facility, University of Oklahoma Health Sciences Center using Applied Biosystems model 377 or ALF automated DNA sequencers. ¹²⁵I Radioactivity was measured using a Packard Auto-Gamma Counting system. Other digital images obtained by scanning blots or autoradiograms with a ScanMaker 9600 XL (MicroTek lab, Inc) were processed using Visioneer Paperport, v5.1 and then Corel Paint or Corel Draw, v9.0.

Cell culture and reagents. MDA-MB-231 and MDA-MB-435 metastatic breast carcinoma cells were maintained in DMEM/Ham's F12 with 5% FBS, and split at 80-90% confluence with 0.05% trypsin. SK-HARE and SK-Hep1 cells were maintained in DMEM with 5% FBS, and split at 80-90% confluence with 0.05% trypsin. Medium for the SK-HARE cells also contained 500 μg/ml geneticin. PC3 and DU145 prostate cancer cells were maintained in F12 with 7% FBS and DMEM with 10% FBS respectively, split at 80-90% confluence with 0.25% trypsin. All cells were maintained at 37° C. and 5% CO₂ and grown in the absence of antibiotics.

Demonstration of tumor and tumor cell associated HA. Tumor cell associated HA was directly demonstrated by peroxidase staining using a biotinylated HA binding probe (Seikagau, Japan) following the manufacturers protocol with and without a Streptomyces hyaluronidase pretreatment to assess specificity. Color was developed with 2% CV/VS aminoethylcarbazole according to the manufacturer instructions, followed by counterstaining with hematoxylin. Tumor cell-associated HA was also indirectly demonstrated in cultured cells with a particle exclusion assay. Glutaraldehyde-fixed sheep red blood cells in PBS/1% BSA were added to cultures of subconfluent carcinoma cells, allowed to settle for 15 min and then observed under phase contrast microscopy. Specificity of the assay was shown by hyaluronidase preteratment of tumor cells.

Assay for functional HARE. Hyaluronan hexylamine derivative (Raja et al., (1984)) was reacted with rhodamine green succinimidyl ester (Molecular Probes, Eugene Oreg.) according to manufacturer's instructions for coupling proteins, quenched, and purified from reactants by gel filtration. The SK-Hep1 cells and SK-HARE tranfectants were incubated at 37° C. with 20 μg/ml of rhodamine green-HA (RG-HA) with or without a 50-fold excess of unlabeled HA for 6 hours.

Cell aggregation assay. SK-HARE and SK-Hep1 cells were labeled with the red fluorescent dye 1,1′-dioctadecyl-3,3,3′3′-tetramethylindocarbocyanine perchlorate (Dil C-18), (Molecular Probes, Eugene, Oreg.) and carcinoma cells were labeled with the green fluorescent dye calcein AM (Molecular Probes) for 40 min, and the labeled cells were harvested from culture dishes by mild trypsinization. Approximately 10⁵ SK-HARE or SK-Hep1 cells were mixed with 10⁵ carcinoma cells and allowed to aggregate for 30 min at 37° C. with gentle mixing. The number of co-aggregates (containing both red and green cells) was assessed in a semi-quantitative manner by counting the distribution of cells in aggregates in 10 separate fields at low magnification (100×) using epi-fluorescence microscopy.

Inhibition of cell aggregation. Cell suspensions labeled with calcein AM were pre-incubated with 16 U/mL Streptomyces hyaluronidase for 1 hour before performing the aggregation assay and hyaluronidase was maintained throughout the assay to remove any HA synthesized by the cells during the assay. Dil C-16-labeled SK-HARE cells were also pre-incubated with 300 μg/ml of exogenous HA (MW˜44,000) which was maintained throughout the aggregation assay to interact with HARE and block its ability to bind HA on the tumor cell surfaces.

Human Metastatic Breast Carcinoma. Cases of breast ductal carcinoma were identified by computer search of the surgical pathology database at the University of Rochester following approval from the Institutional Research Subjects Review Board. The original hematoxylin and eosin stained sections were reviewed and tissue blocks selected for study included the primary breast carcinoma as well as a representative axillary lymph node. The tissue was fixed in 10% neutral buffered formalin and paraffin embedded at the time of original surgery using routine methods. Sections (5 μm) were cut and allowed to dry overnight at 60° C. Paraffin was removed through a series of xylene and alcohol washes, and endogenous peroxidase activity was quenched with 3% hydrogen peroxide. The slides were then subjected to antigen retrieval. Visualization using the anti-HARE antibody mAb#30, and the nonimmune IgG controls, required pepsin digestion for antigen retrieval. The slides were placed in a prewarmed solution of 16 mg of pepsin in 50 ml of 0.1N HCL and incubated at 37° C. for 15 min. The slides for biotinylated-HA binding protein required no antigen retrieval, although a hyaluronidase digestion was employed to assess specificity. The slides were washed with PBS and incubated with the appropriate primary antibody diluted in PBS at room temperature for 60 min. After washing in PBS the slides were treated with biotinylated horse anti-mouse IgG (1:200) for 30 min at room temperature. The slides were then washed with PBS, incubated with streptavidin peroxidase (1:1000), washed once with PBS and once with distilled water and color development was achieved by incubating with 2.0% v/v aminoethylcarbazole and hydrogen peroxide for 5 min according to the manufacture's instructions (ScyTek, Utah). Hematoxylin was used for counterstaining. Slides were viewed with an Olympus BH-2 light microscope equipped with an Olympus 35 mm camera for photomicroscopy.

Materials. ¹²⁵I-HA was prepared using a unique alkylamine derivative of HA (oligosaccharides of Mᵣ˜70,000) as previously described by Raja, et al (1984). Male Sprague-Dawley rats (200 g) were from Charles River Labs. BSA Fraction V was from Intergen Co. The preparation of mouse mAbs against the rat HARE was described by Zhou et al (2000). All other chemical and reagents were from Sigma Chemical Co.

Liver perfusion. Rat livers were removed and perfused ex vivo with Buffer 1 (142 mM NaCl, 6.7 mM KCl, and 10 mM HEPES, pH 7.4) for 8-10 min at ˜35° C. The liver was then perfused by recirculation with 60 ml of medium (GIBCO cat. # 41100) supplemented with 60 mM HEPES, pH 7.4 and 0.1% (w/v) BSA containing 0.25 mg/ml of ¹²⁵I-HA for up to 60 min at ˜35° C. Samples (300 μl) of perfusate were taken at the noted times and divided into 50 μl portions for determination of radioactivity (in duplicate) or degradation (in triplicate). Competitor unlabeled HA (50 μg/ml), purified mAb IgG or mouse IgG (1-5 μg/ml) were added to the perfusion medium containing the ¹²⁵I-HA and mixed well before starting the perfusion. Prior to exposure to the ¹²⁵I-HA, the livers were pre-perfused for 3-25 min with the same concentration of HA or IgG in medium supplemented with 50 μg/ml goat IgG (Sigma cat #1-5256) at ˜35° C.

Degradation of ¹²⁵I-HA. Degradation of ¹²⁵I-HA was measured by a CPC (cetylpyridinium chloride) precipitation assay. Fifty μl portions of perfusion medium containing ¹²⁵I-HA were added (in triplicate) to 250 μl of 1 μg/ml HA (as a carrier) in water in microfuge tubes. Then 300 μl of 6% CPC (in d₂H₂O) was added and the tubes mixed by vortexing. After 10 min at room temperature, the samples were centrifuged in an Eppendorf model 5417 microfuge at room temperature for 5 min at 9000 rpm. Samples (300 μl) were taken, and the remaining supernatants were removed by aspiration. The tips of the tubes were then cut off, put in a gamma counter tube and radioactivity in these and the supernatant samples were determined. Degradation was calculated as the fraction of total radioactivity in each sample that was soluble (non-precipitable). Note that ˜20 to 30% of the radioactivity was not precipitable at the beginning of the experiments.

Selection of stable tranfectants expressing the 175-kDa HARE. SK-Hep-1 cells (from ATCC) were transfected with the purified p175HARE-k DNA using FuGENE 6 in 35 mm culture dishes. Twenty-four h after transfection the cells were transferred to 100 mm dishes and grown in DMEM containing 10% fetal calf serum, L-glutamine, 100 units each of penicillin/streptomycin and 0.4 mg/ml of G418 for selection. After 15-20 days, antibiotic-resistant individual colonies were isolated using cloning rings and detached by treatment with 0.05% trypsin and 0.53 mM EDTA for min at room temperature. Collected cells were expanded in 12-well plates to assess HARE protein expression and function by ELISA, Western blot and ¹²⁵I-HA binding assays. Cultures that were positive in these assays were further purified by dilution cloning. Final clones are designated SK-175HARE4.

Construction of 315 hHARE expression vector. The 315 kDa hHARE cDNA was constructed from two large PCR fragments, comprising the N-terminal upstream coding region (F1) and the 190 coding region (F2). Both fragments overlap by approximately 300 nucleotides, and the complementary strands can thus hybridize and act as both primer and template for the amplification of the full-length 315 kDa hHARE cDNA. The F2 region encodes the full-length 1416 amino acid 190 kDa hHARE. The F2 region was amplified from a previously characterized expression vector (containing the 190 kDa hHARE coding region) using pfu Ultra (Stratagene), gene-specific forward primer (5′-TCCTTACCAAACCTGCTCATGCGGCTGGAACAG-3′) (SEQ ID NO:97) and gene-specific reverse primer (5′-GGATCCCAGTGTCCTCAAGGGGTCATTG-3′) (SEQ ID NO:98). The F1 region was amplified from a Marathon Lymph Node cDNA pool using gene-specific forward primer (5′-GGATCCATGATGCTACAACATTTAGTAATTTTTTGTCTTGG-3′) (SEQ ID NO:99), gene-specific reverse primer (5′-GGTCATTATGGAGAAAGAAGCTCAGGAAATAGGAG-3′) (SEQ ID NO:100), and Advantage 2 polymerase mix (Clontech). The F1 and F2 fragments representing the artificial cDNA were then purified by agarose gel electrophoresis using a 0.8% gel containing 0.002% crystal violet for visualization. The bands were excised, gene-cleaned and subjected to cycles of melting, hybridization, and extension in the absence of other primers using Advantage 2 polymerase mix (35 cycles: 94° C., 20 s; 55° C., 20 s; 68° C., 4.5 min extension time). The resulting artificial cDNA was gel-purified, excised, ligated into the vector, pSecTag/FRT/V5-6×His-TOPO, and transformed into TOP10 E. coli cells. Colonies were selected and screened for the full-length cDNA insert by restriction digestion and PCR analysis, and a correct clone was used to prepare and isolate the expression vector. The 315 hHARE cDNA was then cut out of the plasmid, pSecTag/FRT/V5-6×His-TOPO, and inserted into the BamHI site of pcDNA5/FRT/V5-6×His-TOPO (Invitrogen). This vector is exactly the same as pSecTag/FRT/V5-6×His-TOPO used for the 190 kDa hHARE as previously described (Harris, et al. 2004) except for the absence of the N-terminal Ig kappa secretion signal. Since the full-length 315 kDa hHARE contains a N-terminal membrane insertion signal, the Ig kappa secretion signal is not necessary. After transformation into TOP10 E. coli cells, several clones were selected and the size and orientation of the cDNA insert was verified. The complete DNA sequences of the promoter region, C-terminal fusions for V5 and His-6 epitopes, and the cDNA coding regions of the final clones were determined and confirmed to be correct.

Selection and characterization of stable transfectants expressing the 315-kDa hHARE. Flp-In 293 cells (3×10⁶; from Invitrogen) were plated in 100 mm tissue culture dishes the day prior to transfection. Cells in 10 ml antibiotic-free medium were transfected by addition of 750 μl serum-free DMEM containing 9 μg of pOG44 (which encodes the Flp-In recombinase), 1 μg pcDNA5-315hHARE, and 20 μl Lipofectamine 2000 (Invitrogen). Two days post transfection, the medium was replaced with DMEM containing 100 μg/ml Hygromycin B (Invitrogen). Due to the build-up of dead cells, the medium was changed every 2-3 days. Visible colonies were observed at day 10-14 and then isolated using cloning rings or collected directly with a plastic pipette tip. Isolated colonies were grown to confluence in 24-well dishes in 1.0 ml of DMEM with 100 μg/ml Hygromycin B. After a monolayer of cells had developed from each clone, the cells were scraped and suspended in 1.0 ml of fresh medium. One portion (100 μl) of cells was re-seeded and allowed to grow for subsequent procedures. Another 100 μl of cells was resuspended in DMEM plus 100 μg/ml Zeocin and allowed to grow for one week to test for Zeocin sensitivity. A third portion (400 μl) of cells was pelleted and resuspended in 4× Laemmli (Laemmli, 1970) sample buffer to test for HARE protein expression by SDS-PAGE and Western analysis. The Western blot was probed with anti-V5 antibody (Bethyl Labs; 1:5000 dilution) in TBST. The remaining 400 μl of cell suspension was pelleted and assayed for β-galactosidase activity. Both the Zeocin and β-galactosidase tests indicate whether pcDNA5-315 hHARE was inserted correctly and uniquely into the Flp-In recombination site by the Flp-In recombinase encoded by pOG44. The recombinase is lost during subsequent cell divisions, since the encoding plasmid lacks an antibiotic selection gene. For the β-galactosidase assay, a cell pellet for the clone to be tested was resuspended in 250 μl of 0.5% Triton X-100 in PBS and 10 μl of cell lysate per well (in a 96 well plate) was combined with 20 μl of distilled deionized H₂O, 70 μl Z-buffer (60 mM dibasic sodium phosphate, 40 mM monobasic sodium phosphate, pH 7.0, 10 mM KCl, 1 mM Mg₂SO₄, and 50 mM β-mercaptoethanol), and 20 μl of 4 mg/ml o-nitrophenyl-β-D-galactoside in Z-buffer. After 15 min at 37° C., the enzyme reaction was terminated by the addition of 0.1 ml 1 M sodium bicarbonate and absorbance values were determined at 420 nm. Human embryonic kidney Flp-In 293 cells and 293 cells were included in each assay set as positive and negative controls, respectively. Stable clones with a single plasmid integrated into the correct, unique chromosomal site were those that demonstrated and maintained no detectable β-galactosidase expression, poor or no growth in DMEM containing 100 μg/ml Zeocin, normal cell morphology, and good HARE protein expression. Suitable clones identified were numbers 17.5, 29, 30 and 36 which had β-galactosidase activities (Absorbance per mg protein) of, respectively, 0.008, 0006, 0.012, and 0.004 (these values were the average of duplicates; the negative and positive controls gave values of 0.006 and 1.60, respectively). The 315 hHARE cell lines were maintained in DMEM containing 100 μg/ml Hygromycin B and 8% fetal bovine serum.

Thus it should be apparent that there has been provided in accordance with the present invention a purified nucleic acid segment having a coding region encoding functionally active human HARE or variant thereof, methods of producing HARE or a variant thereof from the HARE gene, methods of purifying HARE or a variant thereof, and the use of fragments or variants of HARE that specifically bind HA, chondroitin and/or chondroitin sulfate as well as antibodies directed thereto, that fully satisfies the objectives and advantages set forth above. Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

### REFERENCES

The following references, to the extent that they provide exemplary procedural or other details supplementary to those set forth herein, are specifically incorporated herein by reference.
1. Abatangelo, G. and Weigel, P. H. (editors) 2000, New Frontiers in Medical Sciences: Redefining Hyaluronan. Elsevier Science B.V. Amsterdam.
2. Aguiar, D. J., W. Knudson, C. B. Knudson 1999. Internalization of the hyaluronan receptor CD44 by chondrocytes, Exp. Cell Res. 252:292-302.
3. Ausubel et al., (eds.), Current Protocols in Molecular Biology, John Wiley & Sons, N.Y. (1989).
4. Auvinen et al. Am. J. Pathol., 156: 529 (2000).
5. Banerji S., Ni J., Wang S., Clasper S., Su J., Tammi R., Jones M., and Jackson D. LYVE-1, a new Homologue of the CD44 Glycoprotein, is a lymph-specific receptor for Hyaluronan. J. Cell Biol. 144:789-801 (1999).
6. Blom et al, J. Molec. Biol. 294:1351 (1999).
7. Bradford, M. M. Anal. Biochem. 72: 248-254 (1976).
8. Bramley, P. N., Rathbone, B. J., Forbes, M. A., Cooper, E. H. and Losowsky, M. S. J. Hepatol. 13: 8-13 (1991).
9. Brodeur et al. Monoclonal Antibody Production Techniques and Applications, Marcel Dekker, Inc., New York, pp. 51-63 (1987).
10. Burd, D. A. R., R. M. Greco, S. Regauer, M. T. Longaker, J. W. Siebert, and H. G. Garg. 1991. Hyaluronan and wound healing: a new perspective. Br. J. Plast. Surg. 44: 579-584.
11. Burnette, W. N. Anal. Biochem. 112:195-203 (1981).
12. Camenisch T., Shroeder J., Bradley J, Klower S., and McDonald J. Heart-valve mesenchyme formation is dependent on HA-augmented activation of ErbB2-ErbB3 receptors. Nat. Med. 8: 850-855 (2002).
13. Cole, et al. In: MONOCLONAL ANTIBODIES AND CANCER THERAPY, Alan R. Liss, Inc., pp. 77-96 (1985).
14. Cote, et al. Proc Natl Acad Sci USA, 80: 2026-2030 (1983).
15. Eriksson, S., Fraser, J. R., Laurent, T. C., Pertoft, H. and Smedsrod, B. Exp. Cell Res. 144: 223-228 (1983).
16. Evered, D., and Whelan, J. (eds) The biology of hyaluronan. Ciba Fnd. Symp. 143: 1-288 (1989).
17. Fishwild et al. Nature Biotechnology, 14: 845-851 (1996).
18. Fraser, J. R., L. E. Appelgren, T. C. Laurent. Tissue uptake of circulating hyaluronic acid. A whole body autoradiographic study. Cell Tissue Res. 233: 285-293 (1983).
19. Fraser, J. R., T. C. Laurent, H. Pertoft, E. Baxter, Plasma clearance, tissue distribution and metabolism of hyaluronic acid injected intravenously in the rabbit, Biochem. J. 200: 415-424 (1981).
20. Freitas, J. P., P. Filipe, I. Emerit, P. Meunier, C. F. Manso, F. Guerra Rodrigo, Hyaluronic acid in progressive systemic sclerosis, Dermatol. 192: 4649 (1996).
21. Gee K, Kryworuchko M, and Kumar A. Recent advances in the regulation of CD44 expression and its role in inflammation and autoimmune diseases. Arch Immunol Ther Exp (Warsz) 52: 13-26 (2004).
22. Ghatak S, Misra S, and Toole B P. Hyaluronan constitutively regulates ErbB2 phosphorylation and signaling complex formation in carcinoma cells. J Biol. Chem. 280(10): 8875-8883 (2005).
23. Goding, Monoclonal Antibodies: Principles and Practice, Academic Press, pp. 59-103 (1986).
24. Goeddel, GENE EXPRESSION TECHNOLOGY: METHODS IN ENZYMOLOGY 185, Academic Press, San Diego, Calif. (1990).
25. Haney, A. F and E. Doty. A barrier composed of chemically cross-linked hyaluronic acid (Incert) reduces post-operative adhesion formation. Fertil Steril. 70: 145-151 (1998).
26. Harris, E. N., Weigel, J. A., and Weigel, P. H. Endocytic function, glycosaminoglycan specificity, and antibody sensitivity of the recombinant human 190 kD HARE (hyaluronan receptor for endocytosis). J. Biol. Chem. 279: 36201-36209 (2004).
27. Hills, B. Boundary lubrication in vivo. Proc. Inst. Mech. Eng. [H] 214:83-94 (2000).
28. Hollyfield J. Hyaluronan and the functional organization of the interphotoreceptor matrix. Invest Ophthalmal. Vis. Sci. 40:2767-2769 (1999).
29. Hoogenboom and Winter. J. Mol. Biol., 227: 381 (1991).
30. Itano, N., Atsumi, F., Sawai, T., Yamada, Y., Miyaishi, O., Senga, T., Hamaguchi, M., and Kimata, K. Abnormal accumulation of hyaluronan matrix diminishes contact inhibition of cell growth and promotes cell migration. Proc Natl Acad Sci USA 99: 3609-3614 (2002).
31. Itano, N., Sawai, T., Miyaishi, O., and Kimata, K. Relationship between hyaluronan production and metastatic potential of mouse mammary carcinoma cells. Cancer Res. 59: 2499-2504 (1999).
32. Jones et al. Nature, 321: 522-525 (1986).
33. Knudson C., and Knudson W. Cartilage Proteoglycans. Semin. Cell. Dev. Biol. 12: 69-78 (2001).
34. Kohler and Milstein, Nature, 256: 495 (1975).
35. Kozbor, et al. Immunol Today, 4: 72 (1983).
36. Kozbor. J. Immunol., 133: 3001 (1984).
37. Kumari, K., Tlapak-Simmons, V. L., Baggenstoss, B. A., and Weigel P. H. The streptococcal hyaluronan synthases are inhibited by sulfhydryl modifying reagents but conserved cysteine residues are not essential for enzyme function. J. Biol. Chem. 277: 13943-13951 (2002).
38. Laemmli, U. K. Nature 227: 680-685 (1970).
39. Lai, K. N., C. C. Szetp. C. W. K. Lam, K. B. Lai, T. Y. H. Wong, J. C. K. Leung, Increased ascitic level of hyaluronan in liver cirrhosis, J. Lab Clin. Med. 131: 354-359 (1998).
40. Lamberg S. and Stoolmiller A. Glycosaminoglycans. A Biochemical and Clinical Review. J. Invest Dermatol. 63: 433-449 (1974).
41. Laurent, C., S. Hellstrom E. Fellenius. Hyaluronan improves the healing of experimental tympanic membrane perforations. A comparison of preparations with different rheologic properties, Arch. Otolaryngol. Head Neck Surg. 114: 1435-1441 (1988).
42. Laurent, T. C., and Fraser, J. R. E. in Degradation of Bioactive Substances: Physiology and Pathophysiology (Henriksen, J. H., ed.), pp. 249-265, CRC Press, Boca Raton, Fla. (1991)
43. Laurent, T. C. and J. R. E. Fraser. Hyaluronan. FASEB J. 6: 2397-2404 (1992).
44. Laurent, U. B., Dahl, L. B. and Reed, R. K. Exp. Physiol. 76: 695-703 (1991).
45. Lebel, L., Smith, L., Risberg, B., Gerdin, B. and Laurent, T. C. J. Appl. Physiol. 64: 1327-1332 (1988).
46. LeBoeuf, R. D., Raja, R. H., Fuller, G. M. and Weigel, P. H. J. Biol. Chem. 261:12586-12592 (1986).
47. LeBoeuf, R. D., Gregg, R. R., Weigel, P. H. and Fuller, G. M. Biochemistry 26: 6052-6057 (1987).
48. Lehr et al. J. Natl. Cancer Inst., 90: 118 (1998).
49. Lokeswar et al. J. Biol. Chem., 275: 641 (2000).
50. Lonberg et al. Nature, 368: 856-859 (1994).
51. Lonberg and Huszar. Intern. Rev. Immunol., 13: 65-93 (1995).
52. Lundin, A., A. Engstrom-Laurent, R. Haligren, G. Michaelsson, Circulating hyaluronate in psoriasis, Br. J. Dermatol. 112: 663-671 (1985).
53. Lynn B., Li X., Cattini P., Turley E., and Nagy J. Identification of Sequence, Protein isoforms, and Distribution of the Hyaluronan-binding protein RHAMM in Adult and Developing Rat Brain. J. Comp. Neurol. 439: 315-330 (2001).
54. Manicourt, D. H., P. Poilvache, A. Nzeusseu, A. van Egeren, J. P. Devogelaer, M. E. Lenz, and E. J. Thonar. Serum levels of hyaluronan, antigenic keratan sulfate, matrix metalloproteinase 3, and tissue inhibitor of metalloproteinases 1 change predictably in rheumatoid arthritis patients who have begun activity after a night of bed rest. Arthritis Rheum. 42:1861-1869 (1999).
55. Marks et al. J. Mol. Biol., 222: 581 (1991).
56. Marks et al. Bio/Technology, 10: 779-783 (1992).
57. McGary, C. T., R. H. Raja, P. H. Weigel, Endocytosis of hyaluronic acid by rat liver endothelial cells. Evidence for receptor recycling, Biochem. J. 257: 875-884 (1989).
58. McGary, C. T., Yannariello-Brown, J., Kim, D. W., Stinson, T. C. and Weigel, P. H. Degradation and intracellular accumulation of a residualizing HA derivative by liver endothelial cells. Hepatology 18: 1465-1476 (1993).
59. Mellman, I. Endocytosis and molecular sorting, Annu. Rev. Cell Biol. 12: 575-625 (1996).
60. Meyer, K. and J W Palmer. The polysaccharide of the vitreous humor. J. Biol. Chem. 107: 629-634 (1934).
61. Mihara et al. Clin. Immunol., 98: 319 (2001).
62. Miletich and Broze. J. Biol. Chem., 265: 11397 (1990).
63. Misra S, Ghatak S, Zoltan-Jones A, and Toole B P. Regulation of multidrug resistance in cancer cells by hyaluronan. J Biol. Chem. 278: 25285-25288 (2003).
64. Morales et al. Nucl. Med. Biol., 27: 199 (2000).
65. Morrison. Nature, 368: 812-813 (1994).
66. Munson and Pollard. Anal. Biochem., 107: 220 (1980).
67. Needleman and Wunsch. J Mol Biol, 48: 443453 (1970).
68. Neuberger. Nature Biotechnology, 14: 826 (1996).
69. Niefind et al. “Amino Acid Similarity Coefficients for Protein Modeling and Sequence Alignment Derived from Main-Chain Folding Anoles.” J. Mol. Biol. 219: 481497 (1991).
70. Okada et al. Clin. Exp. Metastasis, 17: 623 (1999).
71. Oliferenko, S., Kaverina I., Small J., and Huber L. Hyaluronic acid (HA) binding to CD44 activates Rac1 and induces lamellipodia outgrowth. J. Cell Biol 148: 1159-1164 (2000).
72. Overington et al. “Environment-Specific Amino Acid Substitution Tables: Tertiary Templates and Prediction of Protein Folds,” Protein Science 1: 216-226 (1992).
73. Presta. Curr. Op. Struct. Biol., 2: 593-596 (1992).
74. Rahmanian, M., H. Pertoft, S. Kanda, R. Christofferson, L. Claesson-Welsh, and P. Heldin. Hyaluronan oligosaccharides induce tube formation of a brain endothelial cell line in vitro. Exp. Cell Res. 237: 223-230 (1997).
75. Raja, R., LeBoeuf, R., Stone, G. and Weigel, P. Preparation of alkylamine and ¹²⁵-radiolabeled derivatives of hyaluronic acid uniquely modified at the reducing end. Anal. Biochem. 139: 168-177 (1984).
76. Richards et al. Cancer Res., 59: 2096 (1999).
77. Riechmann et al. Nature, 332: 323-327 (1988).
78. Risler et al. “Amino Acid Substitutions in Structurally Related Proteins. A Pattern Recognition Approach.” J. Mol. Biol. 204: 1019-1029 (1988).
79. Sandborn et al. Gastroenterology, 120:1330 (2001).
80. Sanger et al., (1977).
81. Scatchard, G. The attractions of proteins for small molecules and ions. Ann. N Y. Acad. Sci 61: 660-672 (1949).
82. Schultz, J, Milpetz F, Bork P, Ponting C P. SMART, a simple modular architecture research tool: identification of signaling domains. Proc Natl Acad Sci USA 95: 5857-5864 (1998).
83. Seraj et al. Cancer Res., 60: 2764 (2000).
84. Simpson, M. A., Reiland, J., Burger, S. R., Furcht, L. T., Spicer, A. P., Oegema, R. T. Jr., McCarthy, J. B. Hyaluronan synthase elevation in metastatic prostate carcinoma cells correlates with hyaluronan surface retention, a prerequisite for rapid adhesion to bone marrow endothelial cells. J. Biol. Chem. 276: 17949-17957 (2001).
85. Simpson, M. A., Wilson, C. M., Furcht, L. T., Spicer, A. P., Oegema, T. R. Jr., and McCarthy, J. B. Manipulation of hyaluronan synthase expression in prostate adenocarcinoma cells alters pericellular matrix retention and adhesion to bone marrow endothelial cells. J. Biol. Chem. 277:10050-10057 (2002).
86. Slevin, M., J. Krupinski, S. Kumar, and J. Gaffney. Angiogenic oligosaccharides of hyaluronan induce protein tyrosine kinase activity in endothelial cells and activate a cytoplasmic signal transduction pathway resulting in proliferation. Lab Invest. 78: 987-1003 (1998).
87. Smedsrod, B, De Bleser P J, Braet F, Lovisetti P, Vanderkerken K, Wisse E, Geerts A. Cell biology of liver endothelial and Kupffer cells, Gut 35: 1509-1516 (1994).
88. Tammi, R., A. M. Saamanen, H. I. Maibach, and M. Tammi. Degradation of newly synthesized high molecular mass hyaluronan in the epidermal and dermal compartments of human skin in organ culture, J. Invest Dermatol. 97: 126-130 (1991).
89. Tengblad et al., J. Biochem. 236: 521 (1986)
90. Thylen, A., J. Wallin, G. Martensson, Hyaluronan in serum as an indicator of progressive disease in hyaluronan-producing malignant mesothelioma, Cancer 86: 2000-2005 (1999).
91. Tzaicos, C., Fraser, J. R., Tsotsis, E. and Kimpton, W. G. Biochem. J. 264: 823-828 (1989).
92. Verhoeyen et al. Science, 239: 1534-1536 (1988).
93. Weigel, P. H., G. M. Fuller and R. D. LeBoeuf. A model for the role of hyaluronic acid and fibrin in the early events during the inflammatory response and wound healing. J. Theoret. Biol. 119: 219-234 (1986).
94. Weigel, P. H. Subcell. Biochem. 19:125-161 (1993).
95. Weigel, P. H. and Yik, J. H. N. Glycans as endocytosis signals: the cases of the asialoglycoprotein and hyaluronan/chondroitin sulfate receptors. Biochim. Biophys. Acta 1572: 341-363 (2002).
96. Weigel P H, McGary C T, and Weigel J A. The use of iodinated-hyaluronan derivatives to study hyaluronan binding, endocytosis and metabolism by cultured cells. Methods in Enzymol. 363: 382-391 (2003).
97. Weigel, P. H., Ray, D. A. and Oka, J. A. Quantitation of intracellular membrane-bound enzymes and receptors in digitonin-permeabilized cells. Anal. Biochem. 133: 437-449 (1983).
98. West, D. C., I. N. Hampson, F. Arnold, S. Kumar, Angiogenesis induced by degradation products of hyaluronic acid, Science 228:1324-1326 (1985).
99. Yamada, M., Y. Fukuda, I. Nakano, Y. Katano, J. Takamatsu, T. Hayakawa, Serum hyaluronan as a marker of liver fibrosis in hemophiliacs with hepatitis C virus-associated chronic liver disease, Acta Haematol. 99: 212-216 (1998).
100. Yannariello-Brown, J., B. Zhou, D. Ritchie, J. A. Oka, P. H. Weigel, A novel ligand blot assay detects different hyaluronan-binding proteins in rat liver hepatocytes and sinusoidal endothelial cells, Biochem. and Biophys. Res. Commun. 218: 314-319 (1996).
101. Yannariello-Brown, J., B. Zhou, P. H. Weigel, Identification of a 175 kDa protein as the ligand-binding subunit of the rat liver sinusoidal endothelial cell hyaluronan receptor, Glycobiology 7: 15-21 (1997).
102. Yenari et al. Exp. Neurol., 153: 223 (1998).
103. Yenari et al. Neurol. Res., 23: 72 (2001).
104. Yik J H and Weigel P H. The position of cysteine relative to the transmembrane domain is critical for palmitoylation of H1, the major subunit of the human asialoglycoprotein receptor. J Biol Chem 277: 47305-47312 (2002).
105. Yik, J. H. N., Saxena, A., Weigel, J. A., Weigel, P. H. Palmitoylation-defective asialoglycoprotein receptors are normal in their cellular distribution and ability to bind ligand, but are defective in ligand uptake and degradation. Biochem. Biophys. Res. Commun. 297: 980-986 (2002).
106. Yik, J. H. N., Saxena, A., Weigel, J. A., Weigel, P. H. Nonpalmitoylated human asialoglycoprotein receptors recycle constitutively, but are defective in coated pit-mediated endocytosis, dissociation, and delivery of ligand to lysosomes. J. Biol. Chem. 277: 40844-40852 (2002).
107. Zhou, B., Weigel, J. A., Saxena, A., and Weigel, P. H. Molecular cloning and functional expression of the rat 175-kD hyaluronan receptor for endocytosis. Mol. Biol. Cell 13: 2853-2868 (2002).
108. Zhou, B., J. A. Oka, A. Singh, P. H. Weigel, Purification and subunit characterization of the rat liver endocytic hyaluronan receptor, J. Biol. Chem. 274: 33831-33834 (1999).
109. Zhou B, McGary C T, Weigel J A, Saxena A, and Weigel P H. Purification and molecular identification of the human hyaluronan receptor for endocytosis (HARE). Glycobiology 13: 339-349 (2003).
110. Zhou, B., J. A. Weigel, L. Fauss, P. H. Weigel, Identification of the hyaluronan receptor for endocytosis (HARE), J. Biol. Chem. 275: 37733-37741 (2000).

1. A purified recombinant mammalian HARE comprising a polypeptide which is able to specifically bind at least one of HA, chondroitin and chondroitin sulfate, the purified recombinant mammalian HARE comprising at least one of items (a) through (f):
wherein item (a) comprises a purified recombinant mammalian HARE having a molecular weight of about 190 kDa;
wherein item (b) comprises a purified recombinant mammalian HARE having a molecular weight of about 315 kDa;
wherein item (c) comprises a purified recombinant mammalian HARE having an amino acid sequence in accordance with SEQ ID NO:4;
wherein item (d) comprises a purified recombinant mammalian HARE having an amino acid sequence in accordance with SEQ ID NO:96;
wherein item (e) comprises a purified recombinant human HARE; and
wherein item (f) comprises a purified recombinant mammalian HARE which is recognized by at least one of the monoclonal antibodies mAb-30, mAb-154, mAb-159 and a monoclonal antibody which demonstrates an immunological binding characteristic of such monoclonal antibodies.

2. A method of producing a recombinant, functionally active mammalian HARE wherein the recombinant, functionally active HARE is able to specifically bind at least one of HA, chondroitin and chondroitin sulfate, the method comprising the steps of:
providing a recombinant host cell containing a recombinant DNA segment which encodes and is capable of expressing the recombinant mammalian HARE of claim 1; and
culturing the recombinant host cell under conditions that allow for expression of the recombinant DNA segment encoding the functionally active, recombinant mammalian HARE, thereby producing recombinant, functionally active mammalian HARE which is able to specifically bind at least one of HA, chondroitin and chondroitin sulfate.

3. The method of claim 2 further comprising the step of separating and purifying the recombinant, functionally active mammalian HARE from the recombinant host cell.

4. An isolated nucleic acid sequence encoding a functionally active mammalian HARE which is able to specifically bind at least one of HA, chondroitin and chondroitin sulfate, the isolated nucleic acid sequence comprising a nucleic acid sequence in accordance with SEQ ID NO:95.

5. A recombinant vector selected from the group consisting of a plasmid, cosmid, phage, and virus vector and wherein the recombinant vector further comprises the isolated nucleic acid sequence encoding a functionally active mammalian HARE of claim 4.

6. The recombinant vector of claim 5, wherein the recombinant vector is further defined as an expression vector.

7. The recombinant vector of claim 6, wherein the expression vector comprises a promoter operatively linked to the coding region of the mammalian HARE.

8. A recombinant host cell comprising the recombinant vector of claim 5.

9. The recombinant host cell of claim 8, wherein the host cell is further defined as a eucaryotic cell.

10. The recombinant host cell of claim 8, wherein the recombinant host cell produces a functionally active mammalian HARE which specifically binds and endocytoses at least one of HA, chondroitin and chondroitin sulfate.

11. The recombinant host cell of claim 8, wherein the recombinant vector is introduced into the host cell by a method selected from the group consisting of transfection, electroporation, transduction and combinations thereof.

12. The recombinant host cell of claim 8, wherein the purified nucleic acid sequence is integrated into a chromosome of the recombinant host cell.

13. A method of producing a functionally active mammalian HARE which is able to specifically bind at least one of HA, chondroitin and chondroitin sulfate, the method comprising the steps of:
providing the recombinant host cell of claim 8, wherein the recombinant host cell is capable of expressing a functionally active mammalian HARE; and
culturing the recombinant host cell under conditions that allow for expression of the purified nucleic acid sequence encoding a functionally active mammalian HARE, thereby producing a functionally active mammalian HARE which is able to specifically bind at least one of HA, chondroitin and chondroitin sulfate.

14. The method of claim 13 further comprising the step of separating and purifying the functionally active mammalian HARE from the recombinant host cell.

15. An isolated nucleic acid sequence encoding a functionally active variant or fragment of HARE, wherein the functionally active variant or fragment of HARE is able to specifically bind at least one of HA, chondroitin and chondroitin sulfate, the nucleic acid sequence comprising at least one of items (a) through (p):
wherein item (a) comprises a nucleic acid sequence in accordance with SEQ ID NO:55;
wherein item (b) comprises a nucleic acid sequence in accordance with SEQ ID NO:57;
wherein item (c) comprises a nucleic acid sequence in accordance with SEQ ID NO:59;
wherein item (d) comprises a nucleic acid sequence in accordance with SEQ ID NO:61;
wherein item (e) comprises a nucleic acid sequence in accordance with SEQ ID NO:73;
wherein item (f) comprises a nucleic acid sequence in accordance with SEQ ID NO:75;
wherein item (g) comprises a nucleic acid sequence in accordance with SEQ ID NO:77;
wherein item (h) comprises a nucleic acid sequence in accordance with SEQ ID NO:79;
wherein item (i) comprises a nucleic acid sequence in accordance with SEQ ID NO:81;
wherein item (j) comprises a nucleic acid sequence which will hybridize to a complement of at least one of the nucleic acid sequences of items (a)-(i) or a fragment of at least one of the nucleic acid sequences defined in items (a)-(i) under stringent hybridization conditions;
wherein item (k) comprises a nucleic acid sequence that has at least about 76% sequence identity to at least one of the nucleic acid sequences defined in items (a)-(i);
wherein item (l) comprises a nucleic acid sequence that has at least about 80% sequence identity to at least one of the nucleic acid sequences defined in items (a)-(i);
wherein item (m) comprises a nucleic acid sequence that has at least about 85% sequence identity to at least one of the nucleic acid sequences defined in items (a)-(i);
wherein item (n) comprises a nucleic acid sequence that has at least about 90% sequence identity to at least one of the nucleic acid sequences defined in items (a)-(i);
wherein item (o) comprises a nucleic acid sequence that encodes semiconservative or conservative amino acid changes when compared to at least one of the nucleic acid sequences defined in items (a)-(i); and
wherein item (p) comprises a nucleic acid sequence which but for the degeneracy of the genetic code, or encoding of functionally equivalent amino acids, would hybridize to at least one of the nucleic acid sequences defined in items (a)-(i).

16. The isolated nucleic acid sequence of claim 15, wherein the functionally active variant or fragment of HARE encoded by the isolated nucleic acid sequence is soluble.

17. A recombinant vector selected from the group consisting of a plasmid, cosmid, phage, and virus vector and wherein the recombinant vector further comprises the purified nucleic acid sequence encoding a functionally active variant or fragment of HARE of claim 15.

18. The recombinant vector of claim 17, wherein the recombinant vector is further defined as an expression vector.

19. The recombinant vector of claim 17, wherein the expression vector comprises a promoter operatively linked to the coding region of the HARE variant or fragment.

20. The recombinant vector of claim 17, wherein the functionally active variant or fragment of HARE encoded by the purified nucleic acid sequence is soluble.

21. A recombinant host cell comprising the recombinant vector of claim 17.

22. The recombinant host cell of claim 21, wherein the host cell is further defined as a eucaryotic cell.

23. The recombinant host cell of claim 21, wherein the recombinant host cell produces a functionally active variant or fragment of HARE which specifically binds and endocytoses at least one of HA, chondroitin and chondroitin sulfate.

24. The recombinant host cell of claim 21, wherein the recombinant vector is introduced into the host cell by a method selected from the group consisting of transfection, electroporation, transduction and combinations thereof.

25. The recombinant host cell of claim 21, wherein the purified nucleic acid sequence is integrated into a chromosome of the recombinant host cell.

26. The recombinant host cell of claim 21, wherein the functionally active variant or fragment of HARE encoded by the purified nucleic acid sequence is soluble.

27. A method of producing a functionally active variant or fragment of HARE wherein the functionally active variant or fragment of HARE is able to specifically bind at least one of HA, chondroitin and chondroitin sulfate, the method comprising the steps of:
providing the recombinant host cell of claim 21, wherein the recombinant host cell is capable of expressing a functionally active variant or fragment of HARE; and
culturing the recombinant host cell under conditions that allow for expression of the purified nucleic acid sequence encoding a functionally active variant or fragment of HARE, thereby producing a functionally active variant or fragment of HARE which is able to specifically bind at least one of HA, chondroitin and chondroitin sulfate.

28. The method of claim 27 further comprising the step of separating and purifying the functionally active variant or fragment of HARE from the recombinant host cell.

29. The method of claim 27, wherein the functionally active variant or fragment of HARE is soluble.

30. The method of claim 29 further comprising the step of separating and purifying the functionally active soluble variant or fragment of HARE from the recombinant host cell.

31. A purified recombinant mammalian HARE variant or fragment comprising a polypeptide which is able to specifically bind at least one of HA, chondroitin and chondroitin sulfate, the purified recombinant mammalian HARE variant or fragment comprising at least one of items (a) through (p):
wherein item (a) comprises a soluble fragment of HARE;
wherein item (b) comprises an amino acid sequence in accordance with SEQ ID NO:56;
wherein item (c) comprises an amino acid sequence in accordance with SEQ ID NO:58;
wherein item (d) comprises an amino acid sequence in accordance with SEQ ID NO:60;
wherein item (e) comprises an amino acid sequence in accordance with SEQ ID NO:62;
wherein item (f) comprises an amino acid sequence in accordance with SEQ ID NO:74;
wherein item (g) comprises an amino acid sequence in accordance with SEQ ID NO:76;
wherein item (h) comprises an amino acid sequence in accordance with SEQ ID NO:78;
wherein item (i) comprises an amino acid sequence in accordance with SEQ ID NO:80;
wherein item (j) comprises an amino acid sequence in accordance with SEQ ID NO:82;
wherein item (k) comprises an amino acid sequence encoded by a nucleic acid sequence which will hybridize to a complement of a nucleic acid sequence that encodes at least one of the amino acid sequences of items (b)-(j) or a fragment of a nucleic acid sequence that encodes at least one of the amino acid sequences defined in items (b)-(j) under stringent hybridization conditions;
wherein item (l) comprises an amino acid sequence that has at least about 76% sequence identity to at least one of the amino acid sequences defined in items (b)-(j);
wherein item (m) comprises an amino acid sequence that has at least about 80% sequence identity to at least one of the amino acid sequences defined in items (b)-(j);
wherein item (n) comprises an amino acid sequence that has at least about 85% sequence identity to at least one of the amino acid sequences defined in items (b)-(j);
wherein item (o) comprises an amino acid sequence that has at least about 90% sequence identity to at least one of the amino acid sequences defined in items (b)-(j); and
wherein item (p) comprises an amino acid sequence that has semiconservative or conservative amino acid changes when compared to at least one of the amino acid sequences defined in items (b)-(j).

32. A method of producing a functionally active HARE variant or fragment wherein the functionally active HARE variant or fragment is able to specifically bind at least one of HA, chondroitin and chondroitin sulfate, the method comprising the steps of:
providing a recombinant host cell containing a recombinant DNA segment which encodes and is capable of expressing the recombinant mammalian HARE variant or fragment of claim 31; and
culturing the recombinant host cell under conditions that allow for expression of the recombinant DNA segment encoding a recombinant mammalian HARE variant or fragment, thereby producing a recombinant, functionally active mammalian HARE variant or fragment which is able to specifically bind at least one of HA, chondroitin and chondroitin sulfate.

33. The method of claim 32 further comprising the step of separating and purifying the recombinant, functionally active, soluble mammalian HARE variant or fragment from the recombinant host cell.

34. A method of producing a recombinant, functionally active soluble HARE variant or fragment wherein the recombinant, functionally active soluble HARE variant or fragment is able to specifically bind at least one of HA, chondroitin and chondroitin sulfate, the method comprising the steps of:
providing a recombinant host cell containing a recombinant DNA segment which encodes and is capable of expressing a recombinant, functionally active soluble HARE variant or fragment; and
culturing the recombinant host cell under conditions that allow for expression of the recombinant DNA segment encoding a recombinant, functionally active soluble HARE variant or fragment, thereby producing a recombinant, functionally active soluble HARE variant or fragment which is able to specifically bind at least one of HA, chondroitin and chondroitin sulfate.

35. The method of claim 34 further comprising the step of separating and purifying the recombinant, functionally active, soluble HARE variant or fragment from the recombinant host cell.

36. A method of preventing interaction between a first cell expressing HARE on a surface thereof and a second cell whose surface contains at least one of HA, chondroitin and chondroitin sulfate, the method comprising the steps of:
providing a functionally active, soluble variant or fragment of HARE capable of binding at least one of HA, chondroitin and chondroitin sulfate on the surface of the second cell; and
administering an effective amount of the functionally active, soluble variant or fragment of HARE, wherein the functionally active, soluble variant or fragment of HARE inhibits binding of HARE expressed on the surface of the first cell to at least one of HA, chondroitin and chondroitin sulfate on the surface of the second cell.

37. A kit for determining the presence of at least one of HA, heparin, CS-A, CS-B, CS-C, CS-D, CS-E, chondroitin, keratan sulfate, and heparan sulfate, comprising:
at least one variant or fragment of HARE, wherein the at least one variant or fragment of HARE is capable of selectively binding at least one of HA, heparin, CS-A, CS-B, CS-C, CS-D, CS-E, chondroitin, keratan sulfate, and heparan sulfate.

38. The kit of claim 37 wherein the at least one variant or fragment of HARE does not bind at least one of HA, heparin, CS-A, CS-B, CS-C, CS-D, CS-E, chondroitin, keratan sulfate, and heparan sulfate.

39. The kit of claim 37 further comprising a second variant of fragment of HARE, wherein the second variant or fragment of HARE is capable of binding at least one of heparin, CS-A, CS-B, CS-C, CS-D, CS-E, chondroitin, keratan sulfate, and heparan sulfate.

40. The kit of claim 39 wherein the second variant or fragment of HARE does not bind at least one of heparin, CS-A, CS-B, CS-C, CS-D, CS-E, chondroitin, keratan sulfate, and heparan sulfate, and wherein the two variants' inability to bind at least one of heparin, CS-A, CS-B, CS-C, CS-D, CS-E, chondroitin, keratan sulfate, and heparan sulfate is different.
